(19) United States
(12) Patent Application Publication
(10) Pub. No.: US 2022/0298495 A1
(43) Pub. Date: Sep. 22, 2022

(54) NOVEL GENOME EDITING TOOL

(71) Applicant: EmendoBio Inc., Wilmington, DE (US)

(72) Inventors: Lior Izhar, Tel Aviv (IL); Noam Diamant, Kfar Hess (IL); Yuliya Zilberman, Rishon Le Zion (IL)

(73) Assignee: EmendoBio Inc., Wilmington, DE (US)

(21) Appl. No.: 17/617,897

(22) PCT Filed: Jun. 12, 2020
(86) PCT No.: PCT/US20/37560

§ 371 Date: Dec. 9, 2021

Related U.S. Application Data
(60) Provisional application No. 62/860,629, filed on Jun. 12, 2019.
(60) Provisional application No. 63/029,679, filed on May 25, 2020.

(51) Int. Cl. C12N 9/22; C12N 15/11; C12N 15/90

(52) U.S. Cl. CPC C12N 9/22; C12N 15/11; C12N 15/907; C12N 2800/80; C12N 2310/20; C07K 2319/85

(57) ABSTRACT

The present invention provides a novel gene editing composition comprising at least one fusion protein, which comprises a retrotransposon-encoded protein portion linked to a CRISPR nuclease portion, and an RNA template molecule comprising an insert template sequence.

This application claims the benefit of U.S. Provisional Application Nos. 62/860,629 filed Jun. 12, 2019 and 63/029,679 filed May 25, 2020, the contents of which are hereby incorporated by reference.

Throughout this application, various publications are referenced, including referenced in parenthesis. The disclosures of all publications mentioned in this application in their entireties are hereby incorporated by reference into this application in order to provide additional description of the art to which this invention pertains and of the features in the art which can be employed with this invention.

### REFERENCE TO SEQUENCE LISTING

This application incorporates-by-reference nucleotide sequences which are present in the file named “200612_91004-A-PCT_Sequence_Listing_AWG.txt”, which is 203 kilobytes in size, and which was created on Jun. 12, 2020 in the IBM-PC machine format, having an operating system compatibility with MS-Windows, which is contained in the text file filed Jun. 12, 2020 as part of this application.

### BACKGROUND

Targeted genome modification is a powerful tool that can be used to reverse the effect of pathogenic genetic variations and therefore has the potential to provide new therapies for human genetic diseases. Gene editing tools, including engineered zinc finger nucleases (ZFNs), transcription activator-like effector nucleases (TALENs), and more recently, RNA-guided DNA endonuclease systems such as CRISPR/Cas, produce sequence-specific DNA breaks in a genome. The modification of the targeted genomic sequence occurs upon activation of a cellular DNA repair mechanism triggered in response to the newly formed DNA break. Such DNA repair mechanisms can mediate the precise insertion of a sequence that is based on an endogenous or exogenous template molecule at a DNA break site.

Furthermore, a recent gene editing method utilizes a CRISPR nickase-reverse transcriptase fusion protein and an exogenous RNA template to modify a target sequence (Anzalone et al. (2019) “Search-and-replace genome editing without double-strand breaks or donor DNA,” Nature, 576: 149-157). However, simpler template design and diverse fusion protein activities are needed to increase the efficiency, accuracy, and versatility of RNA template-based gene editing.

### SUMMARY OF THE INVENTION

Retrotransposons are preserved genetic elements known for their success in multiplying within many eukaryotic genomes, including the human genome. Reverse transcriptase (RT) activity is central to retrotransposon mobilization, and all autonomous non-LTR retrotransposons encode an RT domain. Also present in many non-LTR retrotransposons is a portion that encodes an endonuclease domain. Furthermore, these retrotransposons also encode for proteins having RNA binding activity and nucleic acid chaperone activity (See Han, “Non-long terminal (non-LTR) retrotransposons: mechanisms, recent developments, and unanswered questions” (2010) Mobile DNA 1 (1):15).

The present invention provides a novel gene editing composition comprising at least one fusion protein, the fusion protein comprising a retrotransposon-encoded protein portion linked to a CRISPR nuclease portion, an RNA template molecule comprising an insert template portion, and an RNA guide molecule that complexes with the CRISPR nuclease portion. The gene editing composition may further comprise an additional retrotransposon-encoded protein.

In some embodiments, the retrotransposon-encoded protein portion of the fusion protein complexes with the RNA template molecule and the CRISPR nuclease portion of the fusion protein complexes with the RNA guide molecule. The formed complex may be utilized as a genome editing tool to modify a desired target sequence according to the RNA template sequence.

According to embodiments of the present invention, there is provided a fusion protein comprising a retrotransposon-encoded protein portion linked by a polypeptide linker to an RNA-guided DNA nuclease portion, e.g. SpCas9. In some embodiments, the retrotransposon-encoded protein portion is N-terminal relative to the RNA-guided DNA nuclease portion. In some embodiments, the retrotransposon-encoded protein portion is C-terminal relative to the RNA-guided DNA nuclease portion.

In some embodiments, the retrotransposon-encoded protein is derived from the non-LTR retrotransposon family, which includes, for example: R2, L1 and I factor proteins.

In some embodiments, the RNA-guided DNA nuclease is a CRISPR nuclease. In some embodiments, the fusion protein of the gene editing composition comprises sequence specific DNA binding protein such as a ZFN fusion protein or a TALENS protein. In some embodiments, the nuclease is a dead nuclease i.e. lacking any DNA nuclease activity. In some embodiments, the nuclease is a nickase, i.e., capable of cutting only a single strand of a double-stranded DNA molecule.

In some embodiments, the retrotransposon-encoded protein reverse transcribes an insert template portion sequence, which leads to subsequent introduction of the reverse-transcribed sequence into a target locus in a mammalian cell, e.g. at a 28S ribosome encoding locus (28S site). In some embodiments, the RNA-guided DNA nuclease portion of the fusion protein is a catalytically dead CRISPR nuclease (e.g. dSpCas9), which targets the fusion protein to a genomic site of interest using an RNA guide molecule, e.g. a single guide RNA (sgRNA) molecule. Advantageously, such a fusion protein displays both CRISPR nuclease target recognition specificity and target-primed reverse transcription (TPRT), thereby providing a dual safety mechanism that greatly reduces off-target effects relative to a CRISPR nuclease alone.

According to some aspects of the invention, there is a provided an RNA molecule comprising (1) a RNA template portion e.g. for editing or correction of an allele; and (2) a retrotransposon protein recruiting portion, which encodes at least one sequence that recruits an endogenous retrotransposon-encoded component (e.g. an L1 protein) reverse-transcribe an RNA template sequence for insertion at a target DNA site. The RNA molecule may further comprise an RNA guide portion which complexes with an RNA-guided DNA nuclease to target the RNA molecule to a target sequence.

In some embodiments, the RNA molecule comprises (1) a RNA guide portion that targets the fusion protein to a target site in the genome; and (2) an RNA template portion. In some embodiments, the RNA molecule comprises (1) a RNA guide portion, which comprises a spacer sequence for targeting a CRISPR nuclease to a genomic target sequence; (2) a scaffold portion for binding a CRISPR nuclease; (3) a retrotransposon-encoded protein binding site (e.g. a R2 protein binding site); (4) an RNA template portion, which encodes a sequence for reverse-transcription and insertion of the reverse transcribed sequence into the genomic target site; and (5) one or two homology arms that share homology with a target locus of a eukaryotic cell (e.g., a plant or mammalian cell). In some embodiments, the RNA molecule further comprises one or more linker sequences, for example, between the RNA template portion and the RNA guide portion.

In an embodiment, the disclosed gene editing composition is delivered as a ribonucleoprotein (RNP) system.

Non-limiting examples of applications of the disclosed genome editing composition include full gene insertion into a target locus, for example a safe harbor site (e.g. a 28S site), insertion of a complete ORF under control of an endogenous promoter upstream of a mutated gene, replacement of a mutated gene sequence with a corrected sequence, and insertion of a promoter and/or an enhancer sequence to promote expression of a silenced gene.

According to some aspects of the invention, there is provided an RNA template molecule comprising:
(1) a retrotransposon-encoded protein binding site, e.g. a site for R2 protein binding and/or L1 protein binding; and
(2) an RNA insert template portion for reverse transcription and insertion or copying of the reverse transcribed template portion into a target site in a gene.

In some embodiments, the RNA template molecule further comprises a homology arm that directs the RNA template molecule to a target site. In some embodiments, the RNA template molecule comprises two homology arms that target the RNA template to a target site. For example, in some embodiments the homology arms direct integration of the reverse transcribed RNA insert template portion to a specific genomic site that shares homology with the homology arms. In some embodiments, the homology arm serves as a primer for target primed reverse transcription of the RNA insert template portion by a retrotransposon-encoded protein at a DNA target site.

According to some aspects of the invention, there is provided a method of altering a target nucleic acid sequence in a cell comprising introducing to the cell an RNA template molecule comprising: (1) a RNA insert template portion for reverse transcription and insertion or copying of the reverse transcribed insert template portion at the target site; and (2) a portion required for binding a retrotransposon-encoded protein, e.g. a R2 protein binding site or a L1 protein binding site.

In some embodiments, the method further comprises introducing to the cell at least one retrotransposon-encoded protein. In some embodiments the retrotransposon-encoded protein is fused to a nuclease. In some embodiments, the nuclease is a CRISPR nuclease or CRISPR nickase. In some embodiments, the CRISPR nuclease is a catalytically inactive or dead nuclease.

In some embodiments, the method further comprises introducing an RNA guide molecule comprising a spacer sequence that targets the CRISPR nuclease to a target nucleic acid site. In some embodiments, the RNA guide molecule comprises a scaffold portion for binding the CRISPR nuclease e.g. a single guide RNA (sgRNA), for example, as described in Jinek et al., “A programmable dual-RNA guided DNA endonuclease in adaptive bacterial immunity.” Science (2012).

In some embodiments, the RNA template molecule is linked to an RNA guide molecule. In some embodiments, a linker polypeptide portion links the RNA template molecule to a sgRNA molecule. Non-limiting examples of an RNA molecule comprising an RNA template portion and a sgRNA portion are provided in SEQ ID NOs: 45-52.

### BRIEF DESCRIPTION OF THE DRAWINGS

FIG. 1A-FIG. 1D: Example schematic representations of the fusion protein and RNA template molecule structure. FIG. 1A—Fusion of a truncated R2 retrotransposon-encoded protein lacking both endonuclease and DNA binding activity, yet retaining reverse transcriptase (RT) and R2 RNA binding activity, fused to a Cas9 nickase. FIG. 1B—Fusion of a truncated R2 retrotransposon-encoded protein unit lacking DNA binding activity yet retaining endonuclease, reverse transcriptase (RT), and R2 RNA binding activity, fused to a dead Cas9 (dCas9). FIG. 1C—An example schematic representation of a synthetic RNA template molecule comprising 5′ and 3′ homology arms, 5′ and 3′ R2 protein binding sites, and an insert template portion for reverse transcription and insertion or copying of the reverse transcribed sequence into a target site. FIG. 1D—An example schematic of the gene editing composition described herein. A black box and a striped box on the genomic DNA indicate that the region shares homology with Homology Arm 1 or Homology Arm 2, respectively, of the RNA template molecule. Optionally, each of the CRISPR nucleases of the depicted fusion proteins may be a DNA nickase or may be catalytically inactivated i.e. a dead nuclease.

FIG. 2: Detection of an insert template sequence insertion upon R2OI protein and RNA template molecule transfection in HeLa cells—R2OI protein construct Kozak-NLS-R2OI-HA-NLS-P2A-mCherry was transfected alone or together with R2OI RNA construct r106-5′UTR-R2OI-3′UTR-r30 into HeLa cells. SpCas9-P2A-mCherry was used as control. Insertion was detected by PCR with Forward Primer 5431 TCGGGTTGCTCTCATCCCTG (SEQ ID NO: 11) binding the C-terminal part of the R2OI ORF and Reverse Primer 5222 CCTCTCATGTCTCTTCACCGTGC (SEQ ID NO: 12) binding the 28S rDNA. A PCR amplicon of the expected 461 bp size was detected only in samples which contained both a protein construct and an RNA template construct.

FIG. 3: SpCas9 functionality upon fusion to transposon protein—HeLa cells were transfected with an EMX guide RNA and either WT SpCas9 or chimera protein constructs as listed on the x-axis. Efficiency of EMX gene editing was measured by NGS and percent editing for each sample in duplicate was calculated using the following formula: (filtered edited reads/total filtered reads)*100%. Each bar shows the average value for two samples, and error bars show the standard deviation.

FIG. 4: Overview of retrotransposition assay—The RNA template is composed of 5′ and 3′ R2OI elements, homology arms targeting a 28S rDNA site (106 bp arm and r30) and an EGFP reporter. The reporter gene contains an antisense copy of the EGFP gene disrupted by Intron 2 of the γ-globin in the sense orientation. The splice donor (SD) and the splice acceptor (SA) sites of the intron are indicated. The EGFP gene is flanked by a PGK promoter (P) and a polyadenylation signal (pA). The transcript originating from a CMV promoter driving the R2OI can splice the intron, but contains the antisense copy of EGFP gene. The EGFP expressing cells will arise only when the transcript is reverse transcribed, integrated into chromosomal DNA, and expressed from the PGK promoter.

FIGS. 5A-5B: R2OI retrotransposon inserts the reporter template into 28S site of rDNA. FIG. 5A—In the first gel (left), samples are loaded as follows: 293T cells transfected with R2OI protein (lanes 1 and 2), reporter (lanes 3 and 4), and R2OI protein and reporter together (lanes 5 and 6). A higher molecular weight band appeared in the reporter sample (lane 4) and in R2OI plus reporter samples (lanes 5 and 6). A lower molecular weight band appeared only in R2OI plus reporter samples (lanes 5 and 6). The second gel (right) shows a nested PCR performed on PCR products from lane 5 of the first gel. In the second gel, band 1 (top) is a non-spliced insert and band 2 is a spliced insert, as confirmed by TA cloning into a pGEM vector and Sanger sequencing. FIG. 5B—Schematic presentation of PCR products obtained in the scenario that the insert contains the non-spliced template (2200 bp) or the spliced insert (1316 bp) product. The forward Primer A anneals to EGFP and the reverse Primer B anneals to the genomic DNA downstream of the insert. The primers are flanking the intron inside the EGFP gene. Primer A (SEQ ID NO: 62), Primer B (SEQ ID NO: 63), and Primer C (SEQ ID NO: 64) are used for sequencing.

### DETAILED DESCRIPTION

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

In the discussion unless otherwise stated, adjectives such as “substantially” and “about” modifying a condition or relationship characteristic of a feature or features of an embodiment of the invention, are understood to mean that the condition or characteristic is defined to within tolerances that are acceptable for operation of the embodiment for an application for which it is intended. Unless otherwise indicated, the word “or” in the specification and claims is considered to be the inclusive “or” rather than the exclusive or, and indicates at least one of and any combination of items it conjoins.

It should be understood that the terms “a” and “an” as used above and elsewhere herein refer to “one or more” of the enumerated components. It will be clear to one of ordinary skill in the art that the use of the singular includes the plural unless specifically stated otherwise. Therefore, the terms “a,” “an” and “at least one” are used interchangeably in this application.

For purposes of better understanding the present teachings and in no way limiting the scope of the teachings, unless otherwise indicated, all numbers expressing quantities, percentages or proportions, and other numerical values used in the specification and claims, are to be understood as being modified in all instances by the term “about.” Accordingly, unless indicated to the contrary, the numerical parameters set forth in the following specification and attached claims are approximations that may vary depending upon the desired properties sought to be obtained. At the very least, each numerical parameter should at least be construed in light of the number of reported significant digits and by applying ordinary rounding techniques.

It is understood that where a numerical range is recited herein, the present invention contemplates each integer between, and including, the upper and lower limits, unless otherwise stated.

In the description and claims of the present application, each of the verbs, “comprise,” “include” and “have” and conjugates thereof, are used to indicate that the object or objects of the verb are not necessarily a complete listing of components, elements or parts of the subject or subjects of the verb. Other terms as used herein are meant to be defined by their well-known meanings in the art.

As used herein, the term “targeting sequence” or “targeting molecule” refers a nucleotide sequence or molecule comprising a nucleotide sequence that is capable of hybridizing to a specific target sequence, e.g., the targeting sequence has a nucleotide sequence which is at least partially complementary to the sequence being targeted along the length of the targeting sequence. For example, the targeting sequence or targeting molecule may be part of a RNA guide molecule that can form a complex with a CRISPR nuclease. When the RNA guide molecule comprising the targeting sequence is present contemporaneously with the CRISPR nuclease, the RNA guide molecule is capable of targeting the CRISPR nuclease to the specific target sequence. In another example, the targeting sequence or targeting molecule may comprise a homology arm that targets a RNA template molecule to a target site for insertion or copying of an insert template sequence at the target site. Each possibility represents a separate embodiment.

As used herein, the term “target” refers to a site comprising a sequence that a targeting molecule shares complementarity with. A target molecule may be designed to specifically target a desired nucleic acid sequence in a genome. It is understood that the term “targets” encompasses variable hybridization efficiencies, such that there is preferential targeting of the nucleic acid having the targeted nucleotide sequence, but unintentional off-target hybridization in addition to on-target hybridization might also occur. It is understood that where an RNA molecule targets a sequence, a complex of the RNA molecule and a CRISPR nuclease molecule targets the entire complex to the target.

As used herein, the term “guide sequence” or “guide portion” of an RNA molecule refers to a nucleotide sequence that is capable of hybridizing to a specific target DNA sequence, e.g., the guide sequence has a nucleotide sequence which is fully complementary to the DNA sequence being targeted along the length of the guide sequence portion. In some embodiments, the guide sequence is 17, 18, 19, 20, 21, 22, 23, 24, or 25 nucleotides in length, or approximately 17-25, 17-24, 17-22, 17-21, 18-25, 18-24, 18-23, 18-22, 18-21, 19-25, 19-24, 19-23, 19-22, 19-21, 19-20, 20-22, 18-20, 20-21, 21-22, or 17-20 nucleotides in length. The entire length of the guide sequence is fully complementary to the DNA sequence being targeted along the length of the guide sequence portion. The guide portion may be part of an RNA molecule that can form a complex with a CRISPR nuclease with the guide portion serving as the DNA targeting portion of the CRISPR complex. When the RNA molecule having the guide portion is present contemporaneously with the CRISPR molecule, the RNA molecule is capable of targeting the CRISPR nuclease to a specific target DNA sequence based on the guide portion sequence. A guide portion can be custom designed to target any desired sequence. Each possibility represents a separate embodiment.

In the context of targeting a DNA sequence that is present in a plurality of cells, it is understood that the targeting encompasses hybridization of the targeting sequence of an RNA molecule with a target sequence in one or more of the cells, and also encompasses hybridization of the targeting sequence of the RNA molecule with the target sequence in fewer than all of the cells in the plurality of cells. For example, it is understood that where an RNA guide molecule targets a sequence in a plurality of cells, a complex of the RNA guide molecule and a CRISPR nuclease is understood to hybridize with the target sequence in one or more of the cells, and also may hybridize with the target sequence in fewer than all of the cells.

As used herein, the term “modified cell” refers to a cell which contains a sequence that has been modified by a gene editing complex as described herein.

The terms “non-naturally occurring” or “engineered” are used interchangeably and indicate human manipulation. The terms, when referring to nucleic acid molecules or polypeptides may mean that the nucleic acid molecule or the polypeptide is at least substantially free from at least one other component with which they are naturally associated in nature and as found in nature.

As used herein, “genomic DNA” refers to linear and/or chromosomal DNA and/or to plasmid or other extrachromosomal DNA sequences present in the cell or cells of interest. In some embodiments, the cell of interest is a eukaryotic cell. In some embodiments, the cell of interest is a prokaryotic cell. In some embodiments, the methods produce double-stranded breaks (DSBs) at pre-determined target sites in a genomic DNA sequence, resulting in mutation, insertion, and/or deletion of DNA sequences at the target site(s) in a genome.

“Eukaryotic” cells include, but are not limited to, fungal cells (such as yeast), plant cells, animal cells, mammalian cells and human cells.

The term “nuclease” as used herein refers to an enzyme capable of cleaving the phosphodiester bonds between the nucleotide subunits of nucleic acid. A nuclease may be isolated or derived from a natural source. The natural source may be any living organism. Alternatively, a nuclease may be a modified or a synthetic protein which retains the phosphodiester bond cleaving activity to create either double or single stranded breaks in DNA, or which has had its nuclease activity completely abolished i.e. a dead nuclease.

The terms “fusion protein” or “chimeric protein” as used herein interchangeably refer to a non-naturally occurring protein in which two or more individual protein portions are linked, preferably covalently. For example, a fusion protein of the present invention comprises a CRISPR nuclease protein portion linked to a retrotransposon-encoded protein portion. The CRISPR nuclease protein portion may comprise, for example, a wild-type CRISPR nuclease, a catalytically inactive CRISPR nuclease, or a CRISPR nickase. Non-limiting examples of a nuclease protein portion are provided in SEQ ID NOs: 40, 41, 53, 54 and 55. The retrotransposon-encoded protein portion may comprise, for example, an R2-encoded protein, an R2OI-encoded protein, or variants thereof. Non-limiting examples of a retrotransposon-encoded protein portion are provided in SEQ ID NOs: 56-69.

The CRISPR nuclease and retrotransposon-encoding protein portions of a fusion protein of the present invention may be in any order in the fusion protein, e.g., the CRISPR nuclease protein portion may be upstream or downstream of the retrotransposon-encoded protein portion (i.e. located in the N-terminal or C-terminal direction from the retrotransposon-encoded protein portion). The fusion protein portions may be linked to each other directly or via a linker, for example, a polypeptide linker. The polypeptide linker connecting the nuclease portion and the retrotransposon-encoded protein portion of the fusion protein may be 5-10, 10-20, 20-50, 50-100, 100-250, 250-500, or up to 1000 amino acids in length or longer. The polypeptide linker may be rigid, flexible, or contain in-vivo cleavage sites. Any polypeptide linker may be used for the construction of the fusion protein. Protein linkers are discussed, for example, in Klein et al. (2014) “Design and characterization of structured protein linkers with differing flexibilities” Protein Eng. Des. Sel. 27 (10) 325-330.

Introduction of a fusion protein in a cell may be the result of delivery of the fusion protein itself to the cell or delivery of a polynucleotide encoding the fusion protein to the cell, wherein the polynucleotide is transcribed and the transcript is translated to generate the fusion protein in the cell. Trans-splicing, polypeptide cleavage and polypeptide ligation can also be involved in expression of the fusion protein in a cell. Methods for polynucleotide and polypeptide delivery to cells are presented elsewhere in this disclosure.

The terms “nuclear localization sequence” and “NLS” are used interchangeably to indicate an amino acid sequence/peptide that directs the transport of a protein with which it is associated from the cytoplasm of a cell across the nuclear envelope barrier. The term “NLS” is intended to encompass not only the nuclear localization sequence of a particular peptide, but also derivatives thereof that are capable of directing translocation of a cytoplasmic polypeptide across the nuclear envelope barrier. NLSs are capable of directing nuclear translocation of a polypeptide when attached to the N-terminus, the C-terminus, or both the N- and C-termini of the polypeptide. In addition, a polypeptide having an NLS coupled by its N-terminus or C-terminus to amino acid side chains located randomly along the amino acid sequence of the polypeptide will be translocated. Typically, an NLS consists of one or more short sequences of positively charged lysines or arginines exposed on the protein surface, but other types of NLS are known.

The terms “RNA template” and “RNA donor”, refer to an RNA molecule that comprises at least one “insert template” portion, which is reverse transcribed into a molecule that is inserted or copied into a genome. Accordingly, the insert template encodes a nucleotide sequence, e.g., of one or more nucleotides, that template a change in the target nucleic acid or are used to modify the target sequence. An insert template may be of any length, for example between 1 and 10,000 nucleotides in length, more preferably between about 10 and 1,000 nucleotides in length.

The RNA template may further comprise additional portions. For example, the RNA template may comprise binding sites for proteins having reverse transcriptase activity to facilitate reverse transcription of the RNA template. For example, the RNA template may comprise a portion, e.g., of one or more nucleotides, that are complimentary to a target nucleic acid site. Such a portion of an RNA template is referred to as a “homology arm.” A homology arm may be 4-10, 10-20, 20-50, 50-100, 100-200, 200-400 nucleotides in length or longer.

An RNA template molecule may be designed, for example, for correction of a mutant gene or for increased expression of a wild-type gene. It will be readily apparent that an insert template portion of an RNA template molecule is typically not identical to a sequence of a genomic target site that the RNA insert template will replace. For example, an RNA template may contain a non-homologous insert template sequence flanked by one or two homology arms, or regions that share homology to a target site, in order to facilitate introduction of the non-homologous insert template sequence at the target site.

An RNA template molecule may be introduced to a cell by expression from a vector, by electroporation into a cell, or introduced via other methods known in the art. An RNA template molecule and be used for gene correction or targeted alteration of an endogenous sequence in a cell. See, for example, U.S. Patent Publication No. 2019/0330620. The RNA template molecule may be used to ‘correct’ a mutated sequence in an endogenous gene (e.g., a sickle-cell causing mutation in beta globin).

An insert template portion of an RNA template molecule may comprise a sequence selected from the group consisting of a gene encoding a protein (e.g., a coding sequence encoding a protein that is lacking in the cell or in the individual or an alternate version of a gene encoding a protein), a regulatory sequence and/or a sequence that encodes a structural nucleic acid such as a microRNA or siRNA. An insert template generally contains at least one sequence difference relative to the target site sequence. Accordingly, the at least one sequence difference is an alteration intended to be introduced into the target site sequence. The at least one difference results in an introduction of a new sequence, deletion of the original target site sequence, or substitution of the target site sequence for a different sequence, or any combination of the above.

An RNA molecule comprising an RNA template portion may further comprise a RNA guide portion. The RNA guide portion may include a scaffold region that binds to a CRIPSR nuclease portion of the inventive fusion protein described herein. The RNA guide portion may be a single guide RNA (sgRNA). An RNA molecule comprising an RNA template portion and a RNA guide portion may be arranged in any conformation, e.g. the RNA template portion may be upstream or downstream of the RNA guide portion. Furthermore, the RNA template portion and the RNA guide portion may be connected to each other by a linker portion. The linker portion may be 1-10, 10-20, 20-50, 50-100 nucleotides in length or longer.

### Fusion Protein Structure

The fusion protein of the gene editing composition may comprise a full-length nuclease portion and a retrotransposon-encoded protein portion, or fragments thereof. The fusion protein portions may be linked to each other directly or via a polypeptide linker. The linker connecting a nuclease portion and a retrotransposon-encoded protein portion of the fusion protein may be 5-10, 10-20, 20-50, 50-100, 100-250, 250-500, or up to 1000 amino acids in length or longer. Any peptide linker can be used for the construction of the fusion protein.

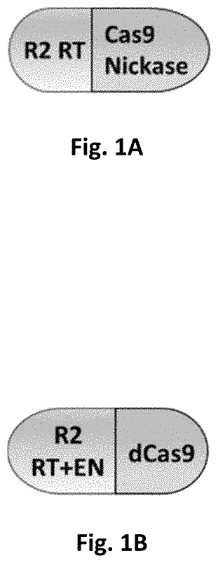

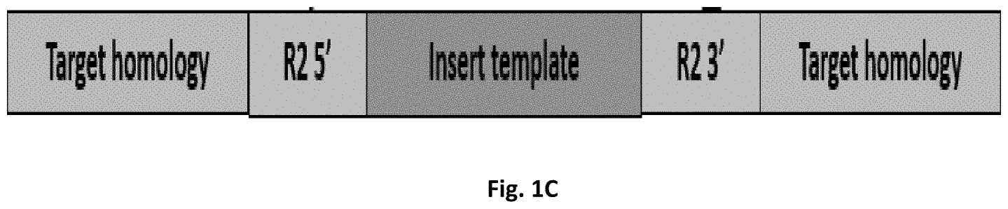

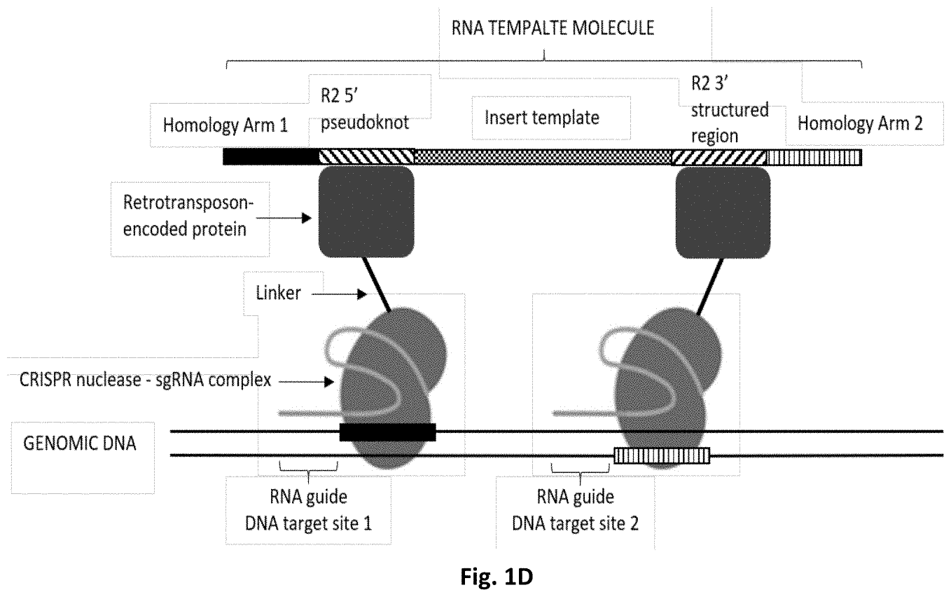

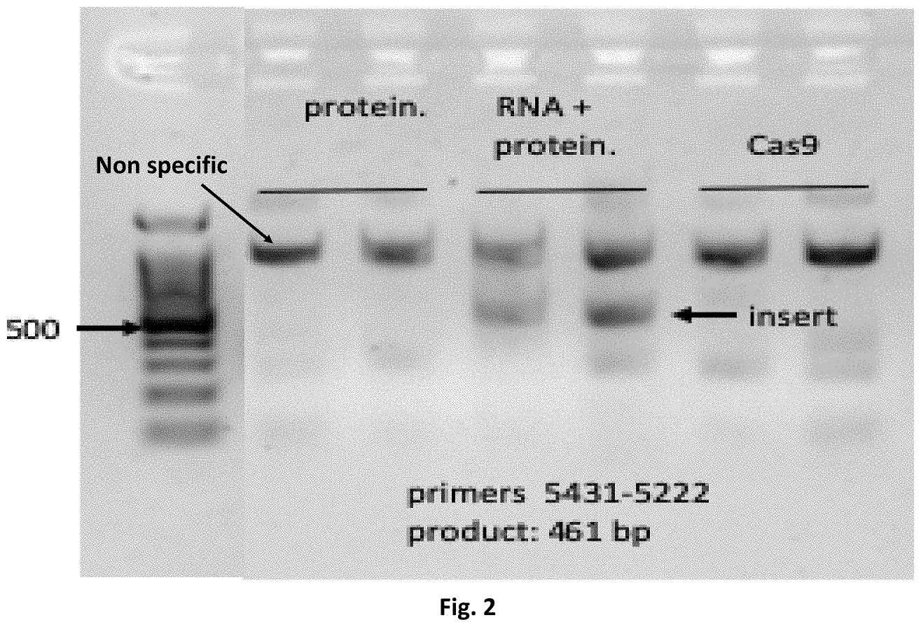

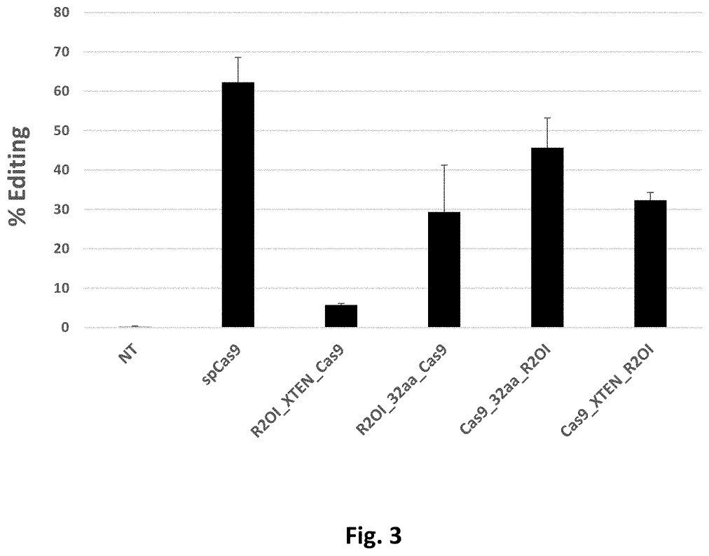

A non-limiting example of a fusion protein compositions includes a fusion protein comprising (1) a truncated R2 retrotransposon-encoded protein unit, wherein the R2 retrotransposon-encoded protein unit lacks both endonuclease and DNA binding activity yet displays reverse transcriptase (RT) and R2 RNA binding activity, fused to (2) a Cas9 nickase unit. See FIG. 1A.

Another non-limiting example of a fusion protein composition includes a fusion protein comprising (1) a truncated R2 retrotransposon-encoded protein unit, wherein R2 retrotransposon-encoded protein unit lacks DNA binding activity yet displays endonuclease, reverse transcriptase (RT), and R2 RNA binding activity, fused to (2) a dead Cas9 (dCas9). See FIG. 1B.

### RNA Template Molecule Structure

As an example, an RNA template molecule includes secondary RNA structures upstream and downstream of an RNA insert template portion. Such RNA structures are used for proper binding of a retrotransposon-encoded protein to the RNA template molecule, and thus serve as retrotransposon-encoded protein binding sites.

In an embodiment, a synthetic RNA template molecule comprises R2 protein binding sites flanking an RNA insert template sequence. The R2 protein binding sites are RNA sequences that form secondary structures which allow R2 protein binding and ribozyme activity. These R2 binding sites are termed the R2 5′ pseudoknot and R2 3′ structured regions. The synthetic RNA template molecule may further comprise one or two homology arms, which are complementary to sequences in a targeted genomic locus and facilitate accurate priming of an RNA template molecule at the genomic locus. The one or two homology arms may be upstream and/or downstream of an R2 protein binding site. See FIG. 1C.

### Function of the Gene Editing Complex

The complex has at least four biochemical activities, including:
1) DNA target site binding mediated by, for example, a CRISPR nuclease;
2) DNA target site cleavage mediated by, for example, a CRISPR nickase, a modified CRISPR nuclease, or a retrotransposon-encoded protein;
3) Binding of an RNA template molecule by a retrotransposon-encoded protein, for example, at binding sites adopted from 5′ and 3′ elements of a R2 RNA; and
4) Reverse transcription, for example, mediated by a retrotransposon-encoded protein.

Fusion protein compositions described herein may be used for introduction of an insert template nucleic acid sequence to a targeted genomic locus in a site-specific manner. This process is completed in several steps:
1) Formation of a complex comprising a fusion protein bound to an RNA molecule comprising an RNA template portion;
2) Introduction of the complex to a cell; and
3) Activity of the complex in the cell occurring in the following steps:
   a) Binding of the complex to a genomic target site;
   b) First strand nicking of the genomic target site;
   c) Target primed reverse transcription of the RNA template;
   d) Second strand nicking of the genomic target site; and
   e) Second strand synthesis.

Alternatively, all components of the complex may be introduced to the cell as one or more DNA constructs capable of producing each component in a cell, or as one or more RNA molecules capable of producing each component in a cell or acting as a component of the complex itself.

### Delivery

The gene editing compositions described herein may be delivered as a protein, DNA molecules, RNA molecules, Ribonucleoproteins (RNP), nucleic acid vectors, or any combination thereof. In some embodiments, the RNA molecule comprises a chemical modification, Non-limiting examples of suitable chemical modifications include 2′-0-methyl (M), 2′-0-methyl, 3′phosphorothioate (MS) or 2′-0-methyl, 3 ‘thioPACE (MSP), pseudouridine, and 1-methyl pseudo-uridine. Each possibility represents a separate embodiment of the present invention.

The gene editing compositions described herein, may be delivered to a target cell by any suitable means. The target cell may be any type of cell e.g., eukaryotic or prokaryotic, in any environment e.g., isolated or not, maintained in culture, in vitro, ex vivo, in vivo or in planta.

Any suitable viral vector system may be used to deliver the compositions disclosed herein. Conventional viral and non-viral based gene transfer methods can be used to introduce the composition in cells (e.g., mammalian cells, plant cells, etc.) and target tissues. Such methods can also be used to administer nucleic acids encoding the composition or the composition itself to cells in vitro. In certain embodiments, the nucleic acids encoding the composition or the composition itself is administered for in vivo or ex vivo gene therapy uses. Non-viral vector delivery systems include naked nucleic acid, and nucleic acid complexed with a delivery vehicle such as a liposome or poloxamer. For a review of gene therapy procedures, see Anderson, Science (1992); Nabel and Felgner, TIBTECH (1993); Mitani and Caskey, TIBTECH (1993); Dillon, TIBTECH (1993); Miller, Nature (1992); Van Brunt, Biotechnology (1988); Vigne et al., Restorative Neurology and Neuroscience 8:35-36 (1995); Kremer and Perricaudet, British Medical Bulletin (1995); Haddada et al., Current Topics in Microbiology and Immunology (1995); and Yu et al., Gene Therapy 1:13-26 (1994).

Methods of non-viral delivery of nucleic acids and/or proteins include electroporation, lipofection, microinjection, biolistics, particle gun acceleration, virosomes, liposomes, immunoliposomes, polycation or lipid:nucleic acid conjugates, artificial virions, and agent-enhanced uptake of nucleic acids or can be delivered to plant cells by bacteria or viruses (e.g., Agrobacterium, Rhizobium sp. NGR234, Sinorhizoboiummeliloti, Mesorhizobium loti, tobacco mosaic virus, potato virus X, cauliflower mosaic virus and cassava vein mosaic virus. See, e.g., Chung et al. Trends Plant Sci. (2006). Sonoporation using, e.g., the Sonitron 2000 system (Rich-Mar) can also be used for delivery of nucleic acids. Cationic-lipid mediated delivery of proteins and/or nucleic acids is also contemplated as an in vivo or in vitro delivery method. See Zuris et al., Nat. Biotechnol. (2015) , Coelho et al., N. Engl. J. Med. (2013); Judge et al., Mol. Ther. (2006); and Basha et al., Mol. Ther. (2011).

Additional exemplary nucleic acid delivery systems include those provided by Amaxa® Biosystems (Cologne, Germany), Maxcyte, Inc. (Rockville, Md.), BTX Molecular Delivery Systems (Holliston, Mass.) and Copernicus Therapeutics Inc., (see for example U.S. Pat. No. 6,008,336). Lipofection is described in e.g., U.S. Pat. Nos. 5,049,386, 4,946,787; and 4,897,355) and lipofection reagents are sold commercially (e.g., Transfectam™, Lipofectin™ and Lipofectamine™ RNAiMAX). Cationic and neutral lipids that are suitable for efficient receptor-recognition lipofection of polynucleotides include those disclosed in PCT International Publication Nos. WO/1991/017424 and WO/1991/016024. Delivery can be to cells (ex vivo administration) or target tissues (in vivo administration).

The preparation of lipid:nucleic acid complexes, including targeted liposomes such as immunolipid complexes, is well known to one of skill in the art (see, e.g., Crystal, Science (1995); Blaese et al., Cancer Gene Ther. (1995); Behr et al., Bioconjugate Chem. (1994); Remy et al., Bioconjugate Chem. (1994); Gao and Huang, Gene Therapy (1995); Ahmad and Allen, Cancer Res., (1992); U.S. Pat. Nos. 4,186,183; 4,217,344; 4,235,871; 4,261,975; 4,485,054; 4,501,728; 4,774,085; 4,837,028; and 4,946,787).

Additional methods of delivery include the use of packaging the nucleic acids to be delivered into EnGeneIC delivery vehicles (EDVs). These EDVs are specifically delivered to target tissues using bispecific antibodies where one arm of the antibody has specificity for the target tissue and the other has specificity for the EDV. The antibody brings the EDVs to the target cell surface and then the EDV is brought into the cell by endocytosis. Once in the cell, the contents are released (see MacDiamid et al., Nature Biotechnology (2009)).

The use of RNA or DNA viral based systems for the delivery of nucleic acids take advantage of highly evolved processes for targeting a virus to specific cells in the body and trafficking the viral payload to the nucleus. Viral vectors can be administered directly to patients (in vivo) or they can be used to treat cells in vitro and the modified cells are administered to patients (ex vivo). Conventional viral based systems for the delivery of nucleic acids include, but are not limited to, retroviral, lentivirus, adenoviral, adeno-associated, vaccinia and herpes simplex virus vectors for gene transfer. However, an RNA virus is preferred for delivery of RNA compositions described herein. Additionally, high transduction efficiencies have been observed in many different cell types and target tissues. Nucleic acid of the invention may be delivered by non-integrating lentivirus. Optionally, RNA delivery with Lentivirus is utilized.

The tropism of a retrovirus can be altered by incorporating foreign envelope proteins, expanding the potential target population of target cells. Lentiviral vectors are retroviral vectors capable of transducing or infecting non-dividing cells and typically produce high viral titers. Selection of a retroviral gene transfer system depends on the target tissue. Retroviral vectors are comprised of cis-acting long terminal repeats with packaging capacity for up to 6-10 kb of foreign sequence. The minimum cis-acting LTRs are sufficient for replication and packaging of the vectors, which are then used to integrate the therapeutic gene into the target cell to provide permanent transgene expression. Widely used retroviral vectors include those based upon murine leukemia virus (MuLV), gibbon ape leukemia virus (GaLV), Simian Immunodeficiency virus (SIV), human immunodeficiency virus (HIV), and combinations thereof (see, e.g., Buchscher Panganiban, J. Virol. (1992); Johann et al., J. Virol. (1992); Sommerfelt et al., Virol. (1990); Wilson et al., J. Virol. (1989); Miller et al., J. Virol. (1991); PCT International Publication No. WO/1994/026877A1).

At least six viral vector approaches are currently available for gene transfer in clinical trials, which utilize approaches that involve complementation of defective vectors by genes inserted into helper cell lines to generate the transducing agent.

pLASN and MFG-S are examples of retroviral vectors that have been used in clinical trials (Dunbar et al., Blood (1995); Kohn et al., Nat. Med. (1995); Malech et al., PNAS (1997)). PA317/pLASN was the first therapeutic vector used in a gene therapy trial. (Blaese et al., Science (1995)). Transduction efficiencies of 50% or greater have been observed for MFG-S packaged vectors. (Ellem et al., Immunol Immunother. (1997); Dranoff et al., Hum. Gene Ther. (1997).

Packaging cells are used to form virus particles that are capable of infecting a host cell. Such cells include 293 cells, which package adenovirus, AAV, and psi.2 cells or PA317 cells, which package retrovirus. Viral vectors used in gene therapy are usually generated by a producer cell line that packages a nucleic acid vector into a viral particle. The vectors typically contain the minimal viral sequences required for packaging and subsequent integration into a host (if applicable), other viral sequences being replaced by an expression cassette encoding the protein to be expressed. The missing viral functions are supplied in trans by the packaging cell line. For example, AAV vectors used in gene therapy typically only possess inverted terminal repeat (ITR) sequences from the AAV genome which are required for packaging and integration into the host genome. Viral DNA is packaged in a cell line, which contains a helper plasmid encoding the other AAV genes, namely rep and cap, but lacking ITR sequences. The cell line is also infected with adenovirus as a helper. The helper virus promotes replication of the AAV vector and expression of AAV genes from the helper plasmid. The helper plasmid is not packaged in significant amounts due to a lack of ITR sequences. Contamination with adenovirus can be reduced by, e.g., heat treatment to which adenovirus is more sensitive than AAV. Additionally, AAV can be produced at clinical scale using baculovirus systems (see U.S. Pat. No. 7,479,554).

In many gene therapy applications, it is desirable that the gene therapy vector be delivered with a high degree of specificity to a particular tissue type. Accordingly, a viral vector can be modified to have specificity for a given cell type by expressing a ligand as a fusion protein with a viral coat protein on the outer surface of the virus. The ligand is chosen to have affinity for a receptor known to be present on the cell type of interest. For example, Han et al., Proc. Natl. Acad. Sci. USA (1995), reported that Moloney murine leukemia virus can be modified to express human heregulin fused to gp70, and the recombinant virus infects certain human breast cancer cells expressing human epidermal growth factor receptor. This principle can be extended to other virus-target cell pairs, in which the target cell expresses a receptor and the virus expresses a fusion protein comprising a ligand for the cell-surface receptor. For example, filamentous phage can be engineered to display antibody fragments (e.g., FAB or Fv) having specific binding affinity for virtually any chosen cellular receptor. Although the above description applies primarily to viral vectors, the same principles can be applied to non-viral vectors. Such vectors can be engineered to contain specific uptake sequences which favor uptake by specific target cells.

Gene therapy vectors can be delivered in vivo by administration to an individual patient, typically by systemic administration (e.g., intravenous, intraperitoneal, intramuscular, subdermal, or intracranial infusion) or topical application, as described below. Alternatively, vectors can be delivered to cells ex vivo, such as cells explanted from an individual patient (e.g., lymphocytes, bone marrow aspirates, tissue biopsy) or universal donor hematopoietic stem cells, followed by reimplantation of the cells into a patient, usually after selection for cells which have incorporated the vector. In some embodiments, delivery of mRNA in-vivo and ex-vivo, and RNPs delivery may be utilized.

Ex vivo cell transfection for diagnostics, research, or for gene therapy (e.g., via re-infusion of the transfected cells into the host organism) is well known to those of skill in the art. In a preferred embodiment, cells are isolated from the subject organism, transfected with an RNA composition, and re-infused back into the subject organism (e.g., patient). Various cell types suitable for ex vivo transfection are well known to those of skill in the art (see, e.g., Freshney, “Culture of Animal Cells, A Manual of Basic Technique and Specialized Applications (6th edition, 2010)) and the references cited therein for a discussion of how to isolate and culture cells from patients).

Suitable cells include but not limited to eukaryotic and prokaryotic cells and/or cell lines. Non-limiting examples of such cells or cell lines generated from such cells include COS, CHO (e.g., CHO-S, CHO-K1, CHO-DG44, CHO-DUXB11, CHO-DUKX, CHOK1SV), VERO, MDCK, WI38, V79, B14AF28-G3, BHK, HaK, NSO, SP2/0-Ag14, HeLa, HEK293 (e.g., HEK293-F, HEK293-H, HEK293-T), and perC6 cells, any plant cell (differentiated or undifferentiated) as well as insect cells such as Spodopterafugiperda (Sf), or fungal cells such as Saccharomyces, Pichia and Schizosaccharomyces. In certain embodiments, the cell line is a CHO-K1, MDCK or HEK293 cell line. Additionally, primary cells may be isolated and used ex vivo for reintroduction into the subject to be treated following treatment with the nucleases (e.g. ZFNs or TALENs) or nuclease systems (e.g. CRISPR). Suitable primary cells include peripheral blood mononuclear cells (PBMC), and other blood cell subsets such as, but not limited to, CD4+ T cells or CD8+ T cells. Suitable cells also include stem cells such as, by way of example, embryonic stem cells, induced pluripotent stem cells, hematopoietic stem cells (CD34+), neuronal stem cells and mesenchymal stem cells.

In one embodiment, stem cells are used in ex vivo procedures for cell transfection and gene therapy. The advantage to using stem cells is that they can be differentiated into other cell types in-vitro or can be introduced into a mammal (such as the donor of the cells) where they will engraft in the bone marrow. Methods for differentiating CD34+ cells in vitro into clinically important immune cell types using cytokines such a GM-C SF, IFN-gamma. and TNF-alpha are known (as a non-limiting example see, Inaba et al., J. Exp. Med. (1992)).

Stem cells are isolated for transduction and differentiation using known methods. For example, stem cells are isolated from bone marrow cells by panning the bone marrow cells with antibodies which bind unwanted cells, such as CD4+ and CD8+ (T cells), CD45+ (panB cells), GR-1 (granulocytes), and Iad (differentiated antigen presenting cells) (as a non-limiting example see Inaba et al., J. Exp. Med. (1992)). Stem cells that have been modified may also be used in some embodiments.

Notably, any one of the compositions described herein may be suitable for genome editing in post-mitotic cells or any cell which is not actively dividing, e.g., arrested cells. Examples of post-mitotic cells which may be edited using a composition of the present invention include, but are not limited to, myocyte, a cardiomyocyte, a hepatocyte, an osteocyte and a neuron.

Vectors (e.g., retroviruses, liposomes, etc.) containing therapeutic compositions can also be administered directly to an organism for transduction of cells in vivo. Alternatively, naked RNA or mRNA can be administered. Administration is by any of the routes normally used for introducing a molecule into ultimate contact with blood or tissue cells including, but not limited to, injection, infusion, topical application and electroporation. Suitable methods of administering such nucleic acids are available and well known to those of skill in the art, and, although more than one route can be used to administer a particular composition, a particular route can often provide a more immediate and more effective reaction than another route.

Vectors suitable for introduction of transgenes into immune cells (e.g., T-cells) include non-integrating lentivirus vectors. See, for example, U.S. Patent Publication No. 2009/0117617.

Pharmaceutically acceptable carriers are determined in part by the particular composition being administered, as well as by the particular method used to administer the composition. Accordingly, there is a wide variety of suitable formulations of pharmaceutical compositions available, as described below (see, e.g., Remington's Pharmaceutical Sciences, 17th ed., 1989).

### CRISPR Nucleases and PAM Recognition

In some embodiments, the RNA-guided DNA nuclease portion of the disclosed fusion protein composition is a CRISPR nuclease, or a functional variant thereof. A skilled artisan will appreciate that RNA guide molecules may be engineered to bind to a target of choice in a genome by commonly known methods in the art.

In embodiments of the present invention, a type II CRISPR system utilizes a mature crRNA:tracrRNA complex directs a CRISPR nuclease, e.g. Cas9, to the target DNA via Watson-Crick base-pairing between the crRNA spacer and the target DNA protospacer sequence next to the protospacer adjacent motif (PAM), which is an additional requirement for target recognition. An active CRISPR nuclease then mediates cleavage of target DNA. A skilled artisan will appreciate that a the guide RNA sequences is designed such as to associate with a target genomic DNA sequence of interest next to a protospacer adjacent motif (PAM), e.g., a PAM corresponding to the type of CRISPR nuclease utilized, such as for a non-limiting example, NGG for Streptococcus pyogenes Cas9 WT (SpCAS9); NNGRRT for Staphylococcus aureus (SaCas9); NNNVRYM for Jejuni Cas9 WT; NGAN or NGNG for SpCas9-VQR variant; NGCG for SpCas9-VRER variant; NGAG for SpCas9-EQR variant; NNNNGATT for Neisseria meningitidis (NmCas9); or TTTV for Cpf1.

In some embodiments, an RNA-guided DNA nuclease e.g., a CRISPR nuclease, may be used to target a desired location in the genome of a cell. The most commonly used RNA-guided DNA nucleases are derived from CRISPR systems, however, other RNA-guided DNA nucleases are also contemplated for use in the genome editing compositions and methods described herein. For instance, see U.S. Patent Publication No. 2015/0211023, incorporated herein by reference.

CRISPR systems that may be used in the practice of the invention vary greatly. CRISPR systems can be a type I, a type II, or a type III system. Non-limiting examples of suitable CRISPR proteins include Cas3, Cas4, Cas5, Cas5e (or CasD), Cash, Cas6e, Cas6f, Cas7, Cas8a1, Cas8a2, Cas8b, Cas8c, Cas9, Cas10, Cas1 Od, CasF, CasG, CasH, Csy1, Csy2, Csy3, Cse1 (or CasA), Cse2 (or CasB), Cse3 (or CasE), Cse4 (or CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csz1, Csx15, Csf1, Csf2, Csf3, Csf4, and Cu1966.

In some embodiments, the RNA-guided DNA nuclease is a CRISPR nuclease derived from a type II CRISPR system (e.g., Cas9). The CRISPR nuclease may be derived from Streptococcus pyogenes, Streptococcus thermophilus, Streptococcus sp., Staphylococcus aureus, Neisseria meningitidis, Treponema denticola, Nocardiopsis dassonvillei, Streptomyces pristinaespiralis, Streptomyces viridochromogenes, Streptomyces viridochromogenes, Streptosporangium roseum, Streptosporangium roseum, Alicyclobacillus acidocaldarius, Bacillus pseudomycoides, Bacillus selenitireducens, Exiguobacterium sibiricum, Lactobacillus delbrueckii, Lactobacillus salivarius, Microscilla marina, Burkholderiales bacterium, Polaromonas naphthalenivorans, Polaromonas sp., Crocosphaera watsonii, Cyanothece sp., Microcystis aeruginosa, Synechococcus sp., Acetohalobium arabaticum, Ammonifex degensii, Caldicelulosiruptor becscii, Candidatus Desulforudis, Clostridium botulinum, Clostridium difjicile, Finegoldia magna, Natranaerobius thermophilus, Pelotomaculumthermopropionicum, Acidithiobacillus caldus, Acidithiobacillus ferrooxidans, Allochromatium vinosum, Marinobacter sp., Nitrosococcus halophilus, Nitrosococcus watsoni, Pseudoalteromonas haloplanktis, Ktedonobacter racemifer, Methanohalobium evestigatum, Anabaena variabilis, Nodularia spumigena, Nostoc sp., Arthrospira maxima, Arthrospira platensis, Arthrospira sp., Lyngbya sp., Microcoleus chthonoplastes, Oscillatoria sp., Petrotoga mobilis, Thermosipho africanus, Acaryochloris marina, or any species which encodes a CRISPR nuclease with a known PAM sequence. CRISPR nucleases encoded by uncultured bacteria may also be used in the context of the invention. (See Burstein et al. Nature, 2017). Variants of CRIPSR proteins having known PAM sequences e.g., SpCas9 D1135E variant, SpCas9 VQR variant, SpCas9 EQR variant, or SpCas9 VRER variant may also be used in the context of the invention.

Thus, an RNA guided DNA nuclease of a CRISPR system, such as a Cas9 protein or modified Cas9 or homolog or ortholog of Cas9, or other RNA guided DNA nucleases belonging to other types of CRISPR systems, such as Cpf1 and its homologs and orthologs, may be used in the compositions of the present invention.

In certain embodiments, the CRIPSR nuclease may be a “functional derivative” of a naturally occurring Cas protein. A “functional derivative” of a native sequence polypeptide is a compound having a qualitative biological property in common with a native sequence polypeptide. “Functional derivatives” include, but are not limited to, fragments of a native sequence and derivatives of a native sequence polypeptide and its fragments, provided that they have a biological activity in common with a corresponding native sequence polypeptide. A biological activity contemplated herein is the ability of the functional derivative to hydrolyze a DNA substrate into fragments. The term “derivative” encompasses both amino acid sequence variants of polypeptide, covalent modifications, and fusions thereof. Suitable derivatives of a Cas polypeptide or a fragment thereof include but are not limited to mutants, fusions, covalent modifications of Cas protein or a fragment thereof. Cas protein, which includes Cas protein or a fragment thereof, as well as derivatives of Cas protein or a fragment thereof, may be obtainable from a cell or synthesized chemically or by a combination of these two procedures. The cell may be a cell that naturally produces Cas protein, or a cell that naturally produces Cas protein and is genetically engineered to produce the endogenous Cas protein at a higher expression level or to produce a Cas protein from an exogenously introduced nucleic acid, which nucleic acid encodes a Cas that is same or different from the endogenous Cas. In some cases, the cell does not naturally produce Cas protein and is genetically engineered to produce a Cas protein.

In some embodiments, the CRISPR nuclease is Cpf1. Cpf1 is a single RNA-guided endonuclease which utilizes a T-rich protospacer-adjacent motif. Cpf1 cleaves DNA via a staggered DNA double-stranded break. Two Cpf1 enzymes from Acidaminococcus and Lachnospiraceae have been shown to carry out efficient genome-editing activity in human cells. See Zetsche et al., (2015) Cell.

Thus, an RNA-guided DNA nuclease of a Type II CRISPR System, such as a Cas9 protein or modified Cas9 or homologs, orthologues, or variants of Cas9, or other RNA guided DNA nucleases belonging to other types of CRISPR systems, such as Cpf1 and its homologs, orthologues, or variants, may be used in a fusion protein of the present invention.

According to embodiments of the present invention, there is provided a gene editing composition comprising an RNA template molecule, at least one fusion protein, and at least one RNA guide molecule,
the RNA template molecule comprising
   a) an insert template portion; and
   b) at least one retrotransposon-encoded protein binding site,
and the at least one fusion protein comprising
   c) at least one retrotransposon-encoded protein portion; and
   d) a CRISPR nuclease portion.

In some embodiments, the RNA template molecule comprises at least one region having sequence homology to a DNA target site.

In some embodiments, the region having sequence homology to a DNA target site flanks a retrotransposon-encoded protein binding site.

In some embodiments, the RNA template molecule comprises a first retrotransposon-encoded protein binding site flanking the 5′ end of the insert template portion, and a second retrotransposon-encoded protein binding site flanking the 3′ end of the insert template portion.

In some embodiments, a first region having sequence homology to a first DNA target site flanks the 5′ end of the first retrotransposon-encoded protein binding site, and a second region having sequence homology to a second DNA target site flanks 3′ end of the second retrotransposon-encoded protein binding site.

In some embodiments, the first retrotransposon-encoded protein binding site is a R2 5′ pseudoknot and the second retrotransposon-encoded protein binding site is a R2 3′ structured region.

In some embodiments, the first RNA guide molecule targets the CRISPR nuclease portion of the first fusion protein to a first CRISPR nuclease DNA target site.

In some embodiments, the RNA template molecule is linked to the first RNA guide molecule. The RNA template molecule may be linked directly to the RNA guide molecule, or may be linked to the RNA guide molecule by an RNA linker portion. The RNA linker portion may be 1-10, 10-20, 20-50, 50-100 or more nucleotides in length.

In some embodiments, the composition further comprises an additional retrotransposon-encoded protein capable of forming a dimer and performing functions of the gene editing process with the retrotransposon-encoded protein of the first fusion protein. In some embodiments, the additional retrotransposon-encoded protein is fused to the retrotransposon-encoded protein portion of the first fusion protein. In some embodiments, the additional retrotransposon-encoded protein is fused to the CRISPR nuclease portion of the first fusion protein.

In some embodiments, the composition further comprises a second fusion protein, the second fusion protein comprising
a) retrotransposon-encoded protein portion; and
b) a CRISPR nuclease protein portion.

In some embodiments, the composition further comprises a second RNA guide molecule that targets the CRISPR nuclease portion of the second fusion protein to a second CRISPR nuclease DNA target site.

In some embodiments, the second CRISPR nuclease DNA target site is within at least 10, 20, 50, 100, 250, 500, or 1000 base pairs of the first CRISPR nuclease DNA target site.

In some embodiments, the CRISPR nuclease portion of the second fusion protein is derived from a species other than the CRISPR nuclease portion of the first fusion protein.

In some embodiments, the retrotransposon-encoded protein of the first or second fusion protein comprises
a) a region that binds a retrotransposon-encoded protein binding site of the RNA molecule; and
b) a reverse transcriptase domain.

In some embodiments, the retrotransposon-encoded protein of the first or second fusion protein further comprises an endonuclease domain.

In some embodiments, the retrotransposon-encoded protein of the first or second fusion protein is derived from a non-LTR retrotransposon-encoded protein.

In some embodiments, the retrotransposon-encoded protein of the first or second fusion protein is derived from an R2, R2OI, L1, or I factor retrotransposon-encoded protein.

In some embodiments, the retrotransposon-encoded protein portion of the first or second fusion protein lacks DNA-binding activity.

In some embodiments, the CRISPR nuclease of the first or second fusion protein is a nickase.

In some embodiments, the CRISPR nuclease of the first or second fusion protein is a catalytically inactive dead CRISPR nuclease.

In some embodiments, the retrotransposon-encoded protein portion and CRISPR nuclease portion of the first or second fusion protein are linked by a polypeptide linker.

In some embodiments, the protein linker is selected from a flexible linker, a rigid linker, and an in-vivo cleavable linker.

In some embodiments, the linker is at least 15 amino acids in length, more preferably at least 30 amino acids in length.

In some embodiments, the linker is an XTEN linker or a 32aa linker.

In some embodiments, the first or second fusion protein comprises the retrotransposon-encoded protein portion linked to the N-terminus of the CRISPR nuclease portion.

In some embodiments, the first or second fusion protein comprises the retrotransposon-encoded protein portion linked to the C-terminus of the CRISPR nuclease portion.

In some embodiments, the first or second fusion protein comprises at least one NLS.

According to embodiments of the present invention, there is provided a polynucleotide molecule which expresses the gene editing composition of any one of the embodiments described herein, or a component thereof, in a cell.

According to embodiments of the present invention, there is provided a method of modifying a sequence at a target site in a eukaryotic cell, the method comprising delivering to the cell the gene editing composition of any one of the embodiments described herein.

In some embodiments, the gene editing composition is delivered to the cell by introducing to the cell a polynucleotide molecule that expresses at least one component of the gene editing composition in the cell.

In some embodiments, the cell is a plant cell or a mammalian cell.

According to embodiments of the present invention, there is provided a modified cell having a sequence that has been modified by the method of any one of the embodiments described herein.

According to embodiments of the present invention, there is provided use of the gene editing composition or a polynucleotide of any one of the embodiments described herein for the treatment of a subject afflicted with a disease or disorder associated with a genomic mutation comprising modifying a nucleotide sequence at a target site in the genome of the subject

According to embodiments of the present invention, there is provided a method of treating subject having a disease or disorder comprising targeting the composition or the polynucleotide of any one of the embodiments described herein to an allele associated with the disease or disorder in a cell of the subject.

According to embodiments of the present invention, there is provided a gene editing composition comprising an RNA molecule comprising an RNA template portion which comprises at least one retrotransposon-encoded protein binding site and an RNA insert template portion.

In some embodiments, the RNA template molecule comprises homology arms flanking an insert template portion.

In some embodiments, the gene editing composition comprises an RNA molecule comprising an RNA template portion and further comprising at least one CRISPR nuclease binding portion. The CRISPR nuclease binding sequence may be part of a tracrRNA or a single guide RNA.

In some embodiments, the RNA molecule further comprises at least one CRISPR guide portion that targets the CRISPR nuclease to a DNA target site.

In some embodiments, the gene editing composition further comprises a retrotransposon-encoded protein portion that forms a complex with the RNA molecule.

The gene editing composition further comprises a CRISPR nuclease, CRISPR nickase, or dead CRISPR nuclease that forms a complex with the RNA molecule.

According to embodiments of the present invention, there is also provided a gene editing composition comprising a fusion protein which comprises a CRISPR protein portion linked to a non-LTR retrotransposon-encoded protein portion. The CRISPR protein portion and non-LTR retrotransposon-encoded protein portions may encode full native proteins or portions thereof.

In some embodiments, the gene editing composition further comprises an RNA molecule which comprises an RNA template portion, wherein the RNA template portion comprises at least one retrotransposon-encoded protein binding site flanking an RNA insert template portion. In some embodiments, the RNA molecule further comprises at least one homology arm flanking the retrotransposon-encoded protein binding site.

In some embodiments, the gene editing composition further comprises at least one RNA molecule comprising an RNA guide which targets a CRISPR nuclease to a target site. In some embodiments, the gene editing composition further comprises a second RNA molecule comprising an RNA guide which targets a CRISPR nuclease to a second target site.

In some embodiments of the gene editing composition, a first fusion protein binds the RNA template at a 5′ retrotransposon-encoded protein binding site of the RNA template, a second fusion protein binds the RNA template at a 3′ retrotransposon-encoded protein binding site of the RNA template, and each CRISPR nuclease portion is bound to a RNA guide molecule.

In some embodiments, a single RNA molecule comprises both an RNA template portion and an RNA guide portion.

According to embodiments of the present invention, there is provided a method of altering a target nucleic acid sequence in a cell comprising introducing into the cell a gene editing composition, wherein a fusion protein of the composition targets a nucleic acid sequence, the fusion protein nicks the target nucleic acid sequence, an RNA insert template sequence is reverse transcribed, and the reverse transcribed RNA insert template sequence is inserted into the targeted nucleic acid sequence.

In an embodiment, the fusion protein comprises one or more of a nuclear localization sequences (NLS), cell penetrating peptide sequences, and/or affinity tags.

In an embodiment, the fusion protein comprises one or more nuclear localization sequences of sufficient strength to drive accumulation of the fusion protein complex into the nucleus of a eukaryotic cell in a detectable amount.

This invention also provides a method of modifying a nucleotide sequence at a target site in a cell-free system or in the genome of a cell comprising introducing into the cell any of the compositions described herein.

In an embodiment, the cell is a eukaryotic cell.

For the foregoing embodiments, each embodiment disclosed herein is contemplated as being applicable to each of the other disclosed embodiment. For example, it is understood that any of the molecules or compositions of the present invention may be utilized in any of the methods of the present invention.

As used herein, all headings are simply for organization and are not intended to limit the disclosure in any manner. The content of any individual section may be equally applicable to all sections.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

Generally, the nomenclature used herein, and the laboratory procedures utilized in the present invention include molecular, biochemical, microbiological and recombinant DNA techniques. Such techniques, used for example, in the design and expression of fusion proteins, are thoroughly explained in the literature. See, for example, Sambrook et al., “Molecular Cloning: A laboratory Manual” (1989); Ausubel, R. M. (Ed.), “Current Protocols in Molecular Biology” Volumes I-III (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (Eds.), “Genome Analysis: A Laboratory Manual Series”, Vols. 1-4, Cold Spring Harbor Laboratory Press, New York (1998); Methodologies as set forth in U.S. Pat. Nos. 4,666,828; 4,683,202; 4,801,531; 5,192,659 and 5,272,057; Cellis, J. E. (Ed.), “Cell Biology: A Laboratory Handbook”, Volumes I-III (1994); Freshney, “Culture of Animal Cells—A Manual of Basic Technique” Third Edition, Wiley-Liss, N.Y. (1994); Coligan J. E. (Ed.), “Current Protocols in Immunology” Volumes I-III (1994); Stites et al. (Eds.), “Basic and Clinical Immunology” (8th Edition), Appleton & Lange, Norwalk, Conn. (1994); Mishell and Shiigi (Eds.), “Strategies for Protein Purification and Characterization—A Laboratory Course Manual” CSHL Press (1996); Clokie and Kropinski (Eds.), “Bacteriophage Methods and Protocols”, Volume 1: Isolation, Characterization, and Interactions (2009), all of which are incorporated by reference. Other general references are provided throughout this document.

Examples are provided below to facilitate a more complete understanding of the invention. The following examples illustrate the exemplary modes of making and practicing the invention. However, the scope of the invention is not limited to specific embodiments disclosed in these Examples, which are for purposes of illustration only.

### EXPERIMENTAL DETAILS

### Example 1—Determining the Insertion Efficiency of R2OI (Medaka fish) and R2 (Bombyx mori) Retrotransposon RNA Templates in Hela Cells at the 28S rDNA Site

### Rationale

A retrotransposon inserts itself into a 28S rDNA site by binding its own RNA, inducing cleavage of the insertion site and performing target-primed reverse transcription (TPRT). A single open reading frame (ORF) encodes for a protein with endonuclease and reverse transcriptase activity. The cleaved DNA strand is used as primer for TPRT. This Example examines activity of retrotransposons R2OI and R2 in a mammalian cell line. In contrast to a native retrotransposon, in this system the RNA and protein components are encoded on two separate vectors.

For the R2OI protein construct, a DNA sequence encoding a R2OI ORF (e.g. ORF encoded by GenBank Accession No. LC349444.1) is codon-optimized for human cells. In this Example, only the R2OI codon-optimized ORF is included in the construct i.e., the 5′UTR and 3′UTR are not included. The DNA sequence is synthesized and inserted into a pcDNA3 backbone as follows: Kozak-NLS-R2OI-HA-NLS-P2A-mCherry.

For the R2OI RNA template constructs, a DNA sequence encoding the R2OI RNA (e.g. encoded by GenBank Accession No. LC349444.1) is inserted into a pTwist vector backbone containing an RNA polymerase I promoter. The DNA sequence encoding the R2OI RNA is synthesized and includes the R2OI 5′UTR (265 bp), ORF (3831 bp) and 3′UTR (108 bp) sequences. To increase the annealing efficiency between the RNA template sequence and the target DNA sequence, a 28S rDNA flanking sequence is added at the 3′ end of the RNA template. To determine the length of the flanking sequence that will show the highest insertion efficiency, a set of R2OI RNA template constructs having rDNA flanking sequences 10 bp, 15 bp, 30 bp and 100 bp in length sequences are utilized. A polymerase I terminator sequence is added after the 3′ homology arm sequence.

R2OI RNA template constructs include:
r106-5′UTR-R2OI-3′UTR-r30;
5′UTR-R2OI-3′UTR-r10;
5′UTR-R2OI-3′UTR-r15;
5′UTR-R2OI-3′UTR-r30; and
5′UTR-R2OI-3′UTR-r100.

For the R2 protein construct, a DNA sequence encoding an R2 ORF (e.g. ORF encoded by GenBank Accession No. M16558.1) is codon-optimized for human cells. In this Example, only the R2 codon-optimized ORF is included in the construct i.e., the 5′UTR and 3′UTR are not included. The DNA sequence is synthesized and inserted into a pcDNA3 backbone as follows: Kozak-NL S-R2-HA-NL S-P2A-mCherry.

For the R2 RNA template constructs, a DNA sequence encoding the R2 RNA (e.g. encoded by GenBank Accession No. M16558.1) is inserted into a pTwist vector backbone containing an RNA polymerase I promoter. The DNA sequence encoding the R2 RNA is synthesized and includes the R2 5′UTR (620 bp), ORF (3345 bp) and 3′UTR (248 bp) sequences. To increase the annealing efficiency between the RNA template sequence and the target DNA sequence, a 28S rDNA flanking sequence is added at the 3′ end of the RNA template. To determine the length of the flanking sequence that will show the highest insertion efficiency, a set of R2 RNA template constructs having rDNA flanking sequences 10 bp, 15 bp, 30 bp and 100 bp in length sequences are utilized.

R2 RNA template constructs include:
5′UTR-R2-3′UTR-r10;
5′UTR-R2-3′UTR-r15;
5′UTR-R2-3′UTR-r30; and
5′UTR-R2-3′UTR-r100.

### Experimental Design

HeLa cells are transfected of with: (1) a R2OI or R2 RNA template construct and (2) the respective R2OI or R2 protein construct. Control samples are transfected with either an RNA template construct or a protein construct only. Genomic DNA is isolated 72 hours after transfection. Insertion efficiency is measured by digital droplet PCR (ddPCR) with a forward primer binding to the 3′ end of the insert DNA and a reverse primer binding to the 28S genomic sequence. Alternatively, a R2OI or R2 RNA template and a R2OI or R2 protein encoding transcript are transcribed in vitro and transfected into Hela as RNA.

### Results

The R2OI protein construct Kozak-NLS-R2OI-HA-NLS-P2A-mCherry was transfected in HeLa cells alone or together with R2OI RNA template construct r106-5′UTR-R2OI-3′UTR-r30. SpCas9-P2A-mCherry was used as a control. Insertion was detected by PCR with the following primers: Forward Primer 5431 TCGGGTTGCTCTCATCCCTG (SEQ ID NO: 11), which binds the C-terminal part of the R2OI ORF; and Reverse Primer 5222 CCTCTCATGTCTCTTCACCGTGC (SEQ ID NO: 12), which binds the 28S rDNA. A PCR amplicon of the expected 461 bp size was detected only in samples which contained both a protein construct and an RNA template construct.

### Example 2—Determining SpCas9 Functionality Upon Fusion to a R2OI Retrotransposon-Encoded Protein

### Rationale

An aspect of the invention provides for a chimera protein comprising a retrotransposon-encoded protein and dead-SpCas9 (dSpCas9), where a retrotransposon-encoded protein will perform endonuclease and reverse transcriptase functions, and the dSpCas9 complexed with a guide RNA will target the entire chimera protein complex to a specific genomic site. Thus, the first step was to test if SpCas9 is able to target to a specific genomic site when fused to retrotransposon-encoded protein, which itself is similar in size to SpCas9. Here N-terminal and C-terminal SpCas9 fusion protein conformations, as well as two different linkers, XTEN (See V. Schellenberger et al., Nature Biotechnology, 2009) and 32aa linker (See T. P. Huang and K. T. Zhao, Nature Biotechnology, 2019), are tested. Wild type SpCas9-P2A-mCherry is used as control.

Protein chimera constructs include:
Kozak-NLS-R2OI-HA-NLS-XTEN-SpCas9;
Kozak-NLS-R2OI-HA-NLS-32aa-SpCas9;
Kozak-SpCas9-NLS-XTEN-R2OI-HA-NLS; and
Kozak-SpCas9-NLS-32aa-R2OI-HA-NLS.

### Experimental Design

HeLa cells seeded in a 96-well plate were transfected with (1) a guide RNA targeting the EMX gene; and (2) a WT SpCas9 or chimera protein construct. Genomic DNA was isolated 72 hours after transfection. Efficiency of EMX gene editing was measured by NGS and percent editing for each sample in duplicate was calculated as follows: (filtered edited reads/total filtered reads)*100%. Samples for Illumina NGS analysis were prepared using a Nextera DNA XT Library Prep Kit according to the manufacturer's protocol.

### Results

Percent editing was the highest for the chimera with the longer linker SpCas9-NLS-32aa-R2OI-HA-NLS (45%), compared to control WT SpCas9 (62%). The lowest percent editing was measured in the chimera with the XTEN linker and SpCas9 fused at the N-terminus, NLS-R2OI-HA-NLS-XTEN-SpCas9 (5.7%). The other two chimeras displayed similar percent editing to SpCas9, measuring 29% for NLS-R2OI-HA-NLS-32aa-SpCas9 and 32% for SpCas9-NLS-XTEN-R2OI-HA-NLS.

### Example 3—Determine R2OI Retrotransposon-Encoded Protein Activity Upon Fusion to Dead-SpCas9

### Rationale

As described in Example 2, an aspect of the invention provides for a gene-editing tool comprising a retrotransposon-encoded protein and dCas9 chimera protein. Thus, R2 and R2OI proteins were tested to determine which retrotransposon-encoded protein displays superior activity at its home site, the 28S rDNA locus, in the context of a chimera protein with dCas9. Chimera proteins comprising an XTEN or 32aa linker are tested. Additionally, both N-terminal and C-terminal fusions to dSpCas9 are tested.

Protein chimera constructs include:
Kozak-NLS-R2OI-HA-NLS-XTEN-dead_SpCas9;
Kozak-NLS-R2OI-HA-NLS-32aa-dead_SpCas9;
Kozak-dead_SpCas9-NLS-XTEN-R2OI-HA-NLS;
Kozak-dead_SpCas9-NLS-32aa-R2OI-HA-NLS;
Kozak-NLS-R2-HA-NLS-XTEN-dead_SpCas9;
Kozak-NLS-R2-HA-NLS-32aa-dead_SpCas9;
Kozak-dead_SpCas9-NLS-XTEN-R2-HA-NLS; and
Kozak-dead_SpCas9-NLS-32aa-R2-HA-NLS.

### Experimental Design

HeLa cells seeded in a 96-well plate are transfected with an RNA template construct and a protein chimera construct. Genomic DNA is isolated 72 hours after transfection. Insertion efficiency is measured by ddPCR with a forward primer binding to the 3′ end of the insert DNA and a reverse primer binding to the 28S rDNA genomic sequence. Ability of the chimera to insert the RNA template at a target site is compared to a retrotransposon-encoded protein alone.

### Example 4—Genomic Insertion of a Reverse Transcription Reporter RNA by R2OI

### Rationale

Previous examples demonstrated that the R2OI retrotransposon is able to insert itself at a 28S site of rDNA in HeLa cells. This Example tests the activity of a native R2OI (Medaka fish) retrotransposon in human cells by utilizing a reporter system to further demonstrate that the insertions are not due to homologous recombination (HR) between the vector DNA and the genomic DNA.

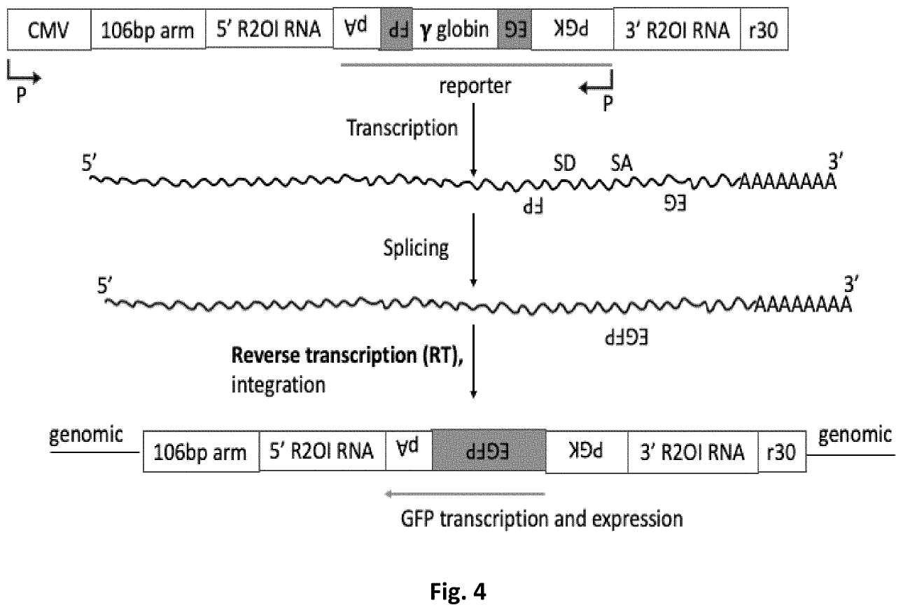

In order to distinguish between HR and insertion by target-primed reverse transcription (TPRT), a reporter system that was developed for human L1 retrotransposon was adopted (Moran et al. “High frequency retrotransposition in cultured mammalian cells” (1996) Cell, 87:917-927). The reporter cassette consists of an antisense copy of the EGFP gene, a hPGK promoter, and a polyadenylation signal. The EGFP gene is disrupted by an intron in the opposite transcriptional orientation (FIG. 4). This set-up ensures that EGFP-expressing cells will arise only when a transcript initiated from a promoter driving R2OI RNA is spliced, reverse transcribed, reintegrated into chromosomal DNA, and expressed from the PGK promoter. In this arrangement transcripts originating from the vector encoded PGK promoter cannot be spliced, the and EGFP product will not be synthesized.

An RNA template-encoding construct was inserted into the pcDNA3.1 vector backbone with a CMV promoter. The sequence comprises a 106 bp homology arm, R2OI_5′R2OI RNA, an hPGK-GFP reporter cassette, R2OI_3′ R2OI RNA, and a r30—30 bp homology arm (FIG. 4).

### Experimental Design

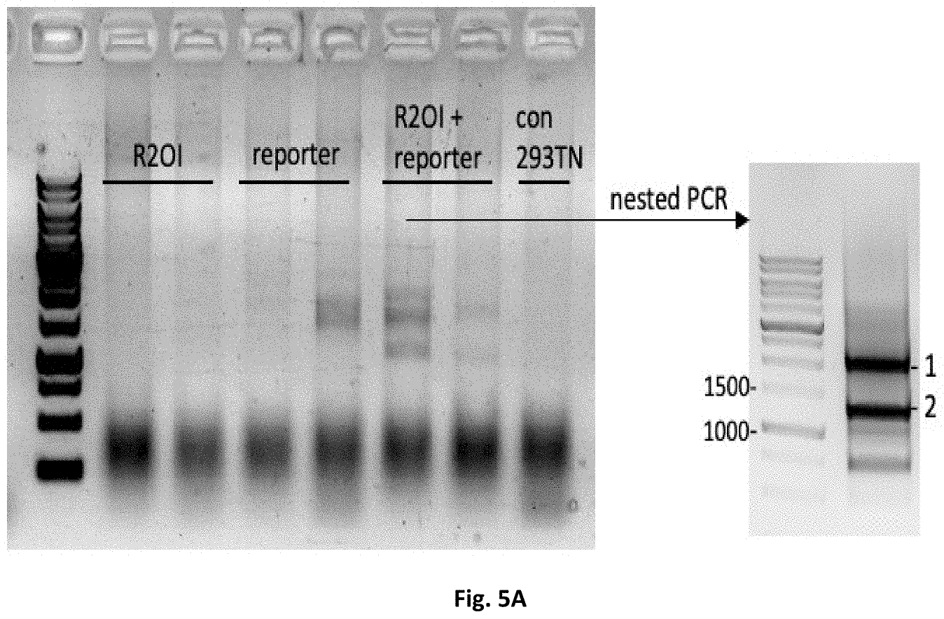

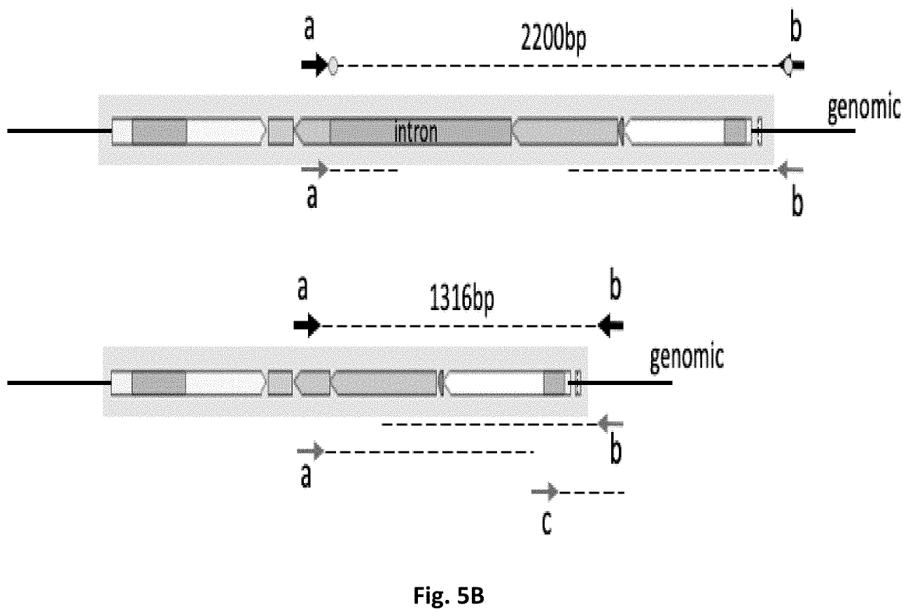

293TN cells were transfected with a reporter RNA-encoding construct and R2OI protein construct (Kozak-NLS-R2-HA-NLS-P2A-mCherry). In control samples, an R2OI protein construct and an RNA construct were transfected separately. Genomic DNA was isolated 72 hours post-transfection. Insert was detected by PCR with forward primer TGCTCAGGTAGTGGTTGTCG (SEQ ID NO: 70), which anneals to EGFP upstream of an intron, and reverse primer CCTCTCATGTCTCTTCACCGTGC (SEQ ID NO: 12), which anneals to a genomic DNA site downstream of the insert. A product of about 2000 bp was detected in samples transfected with reporter RNA only, while three PCR products were detected in samples transfected with both constructs. In order to increase specificity, nested PCR was performed on a sample transfected with both protein and RNA constructs using forward Primer A: CTCAGGTAGTGGTTGTCGGGC (SEQ ID NO: 62) and reverse Primer B: GGACAGTGGGAATCTCGTTC (SEQ ID NO: 63). Nested PCR products were separated by gel electrophoreses (FIG. 5A). Two bands from the gel were excised, purified, and sequenced. Additionally, the PCR products were cloned into a pGEM vector and sequenced again to confirm that a single PCR product was the template for all primers.

### Results

The sequencing results revealed that the top band (1) is the non-spliced insert (2200 bp) and the lower band (2) is the spliced insert (FIG. 5A). It is concluded that the reporter tool allows for distinguishing between HR and TPRT and that the R2OI retrotransposon is functional in human cells and is able to insert foreign DNA at a 28S rDNA site in the genome.

1. A gene editing composition comprising an RNA template molecule, at least one fusion protein, and at least one RNA guide molecule,
the RNA template molecule comprising
a) an insert template portion; and
b) at least one retrotransposon-encoded protein binding site,
and the at least one fusion protein comprising
c) at least one retrotransposon-encoded protein portion; and
d) a CRISPR nuclease portion.

2. The gene editing composition of claim 1, wherein the RNA template molecule further comprises at least one region having sequence homology to a DNA target site.

3. The gene editing composition of claim 1, wherein the region having sequence homology to a DNA target site flanks a retrotransposon-encoded protein binding site.

4. The gene editing composition of claim 1, wherein the RNA template molecule comprises a first retrotransposon-encoded protein binding site flanking the 5′ end of the insert template portion, and a second retrotransposon-encoded protein binding site flanking the 3′ end of the insert template portion.

5. The gene editing composition of claim 4, wherein a first region having sequence homology to a first DNA target site flanks the 5′ end of the first retrotransposon-encoded protein binding site, and a second region having sequence homology to a second DNA target site flanks 3′ end of the second retrotransposon-encoded protein binding site, and/or
wherein the first retrotransposon-encoded protein binding site is a R2 5′ pseudoknot and the second retrotransposon-encoded protein binding site is a R2 3′ structured region.

6. (canceled)

7. The gene editing composition of claim 1, wherein the first RNA guide molecule targets the CRISPR nuclease portion of the first fusion protein to a first CRISPR nuclease DNA target site, and/or
wherein the RNA template molecule is linked to the first RNA guide molecule.

8. (canceled)

9. The gene editing composition of claim 1, further comprising a second fusion protein, the second fusion protein comprising
a) a retrotransposon-encoded protein portion; and
b) a CRISPR nuclease protein portion.

10. The gene editing composition of claim 9, further comprising a second RNA guide molecule that targets the CRISPR nuclease portion of the second fusion protein to a second CRISPR nuclease DNA target site and the second CRISPR nuclease DNA target site is within at least 10, 20, 50, 100, 250, 500, or 1000 base pairs of the first CRISPR nuclease DNA target site.

11. (canceled)

12. The gene editing composition claim 9, wherein the CRISPR nuclease portion of the second fusion protein is derived from a species other than the CRISPR nuclease portion of the first fusion protein.

13. The gene editing composition of claim 1, wherein the retrotransposon-encoded protein of the fusion protein comprises
a) a region that binds a retrotransposon-encoded protein binding site of the RNA molecule; and
b) a reverse transcriptase domain.

14. The gene editing composition of claim 1, wherein the retrotransposon-encoded protein of the fusion protein further comprises an endonuclease domain.

15. The gene editing composition of claim 1, wherein the retrotransposon-encoded protein of the fusion protein is derived from a non-LTR retrotransposon-encoded protein, or
wherein the retrotransposon-encoded protein of the fusion protein is derived from an R2, R2OI, L1, or I factor retrotransposon-encoded protein, and/or
wherein the retrotransposon-encoded protein portion of the fusion protein lacks DNA-binding activity.

16. (canceled)

17. (canceled)

18. The gene editing composition of claim 1, wherein the CRISPR nuclease of the fusion protein is a nickase, or the CRISPR nuclease of the fusion protein is a catalytically inactive dead CRISPR nuclease.

19. (canceled)

20. The gene editing composition of claim 1, wherein the retrotransposon-encoded protein portion and CRISPR nuclease portion of the fusion protein are linked by a polypeptide linker.

21. The gene editing composition of claim 20, wherein the protein linker is selected from a flexible linker, a rigid linker, and an in-vivo cleavable linker, and/or
the linker is at least 15 amino acids in length, more preferably at least 30 amino acids in length, and/or
the linker is an XTEN linker or a 32aa linker.

22. (canceled)

23. (canceled)

24. The gene editing composition of claim 1, wherein the fusion protein comprises the retrotransposon-encoded protein portion linked to the N-terminus of the CRISPR nuclease portion, or
wherein the fusion protein comprises the retrotransposon-encoded protein portion linked to the C-terminus of the CRISPR nuclease portion, and/or
wherein the fusion protein comprises at least one nuclear localization signal (NLS).

25. (canceled)

26. (canceled)

27. A polynucleotide molecule which expresses the gene editing composition of claim 1, or a component thereof, in a cell.

28. A method of modifying a sequence at a target site in a eukaryotic cell, the method comprising delivering to the cell the gene editing composition of claim 1, or wherein the gene editing composition is delivered to the cell by introducing to the cell a polynucleotide molecule that expresses at least one component of the gene editing composition in the cell, and wherein the cell is a plant cell or a mammalian cell.

29. (canceled)

30. (canceled)

31. A modified cell having a sequence that has been modified by the method of claim 28.

32. (canceled)

33. A method of treating subject having a disease or disorder comprising targeting the composition of claim 1 to an allele associated with the disease or disorder in a cell of the subject.


SEQUENCE LISTING (72 sequences)

SEQ ID NO: 1 - protein, 10 aa, Artificial Sequence
  SEQ ID NO 1 - BE4 short linker
SGGSGGSGGS

SEQ ID NO: 2 - protein, 32 aa, Artificial Sequence
  SEQ ID NO 2 - BE4 long linker
SGGSSGGSSGSETPGTSESATPESSGGSSGGS

SEQ ID NO: 3 - protein, 1368 aa, Artificial Sequence
  SEQ ID NO 3 - Dead SpCas9
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 4 - protein, 1368 aa, Artificial Sequence
  SEQ ID NO 4 - D10A Nickase SpCas9
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 5 - protein, 1368 aa, Artificial Sequence
  SEQ ID NO 5 - H840A SpCas9 nickase
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 6 - protein, 1114 aa, Artificial Sequence
  SEQ ID NO 6 - Bm R2 CDS
MMASTALSLMGRCNPDGCTRGKHVTAAPMDGPRGPSSLAGTFGWGLAIPAGEPCGRVCSPATVGFFPVAKKSNKENRPEASGLPLESERTGDNPTVRGSAGADPVGQDAPGWTCQFCERTFSTNRGLGVHKRRAHPVETNTDAAPMMVKRRWHGEEIDLLARTEARLLAERGQCSGGDLFGALPGFGRTLEAIKGQRRREPYRALVQAHLARFGSQPGPSSGGCSAEPDFRRASGAEEAGEERCAEDAAAYDPSAVGQMSPDAARVLSELLEGAGRRRACRAMRPKTAGRRNDLHDDRTASAHKTSRQKRRAEYARVQELYKKCRSRAAAEVIDGACGGVGHSLEEMETYWRPILERVSDAPGPTPEALHALGRAEWHGGNRDYTQLWKPISVEEIKASRFDWRTSPGPDGIRSGQWRAVPVHLKAEMFNAWMARGEIPEILRQCRTVFVPKVERPGGPGEYRPISIASIPLRHFHSILARRLLACCPPDARQRGFICADGTLENSAVLDAVLGDSRKKLRECHVAVLDFAKAFDTVSHEALVELLRLRGMPEQFCGYIAHLYDTASTTLAVNNEMSSPVKVGRGVRQGDPLSPILFNVVMDLILASLPERVGYRLEMELVSALAYADDLVLLAGSKVGMQESISAVDCVGRQMGLRLNCRKSAVLSMIPDGHRKKHHYLTERTFNIGGKPLRQVSCVERWRYLGVDFEASGCVTLEHSISSALNNISRAPLKPQQRLEILRAHLIPRFQHGFVLGNISDDRLRMLDVQIRKAVGQWLRLPADVPKAYYHAAVQDGGLAIPSVRATIPDLIVRRFGGLDSSPWSVARAAAKSDKIRKKLRWAWKQLRRFSRVDSTTQRPSVRLFWREHLHASVDGRELRESTRTPTSTKWIRERCAQITGRDFVQFVHTHINALPSRIRGSRGRRGGGESSLTCRAGCKVRETTAHILQQCHRTHGGRILRHNKIVSFVAKAMEENKWTVELEPRLRTSVGLRKPDIIASRDGVGVIVDVQVVSGQRSLDELHREKRNKYGNHGELVELVAGRLGLPKAECVRATSCTISWRGVWSLTSYKELRSIIGLREPTLQIVPILALRGSHMNWTRFNQMTSVMGGGVG

SEQ ID NO: 7 - DNA, 75 bp, Artificial Sequence
  SEQ ID NO 7 - 5′ R2 RNA pseudoknot
gccccgatggacggaccgcgaggaccgtcaagcctagcaggtaccttcgggtggggccttgcgatacctgcgggc

SEQ ID NO: 8 - DNA, 248 bp, Artificial Sequence
  SEQ ID NO 8 - 3′ R2 RNA structured region
gccttgcacagtagtccagcggtaagggtgtagatcaggcccgtctgtttctcccccggagctcgctcccttggcttcccttatatattttaacatcagaaacagacattaaacatctactgatccaatttcgccggcgtacggccacgatcgggagggtgggaatctcgggggtcttccgatcctaatccatgatgattacgacctgagtcactaaagacgatggcatgatgatccggcgatgaaaa

SEQ ID NO: 9 - protein, 398 aa, Artificial Sequence
  SEQ ID NO 9 - R2 RNA BD + RT domain
RAEYARVQELYKKCRSRAAAEVIDGACGGVGHSLEEMETYWRPILERVSDAPGPTPEALHALGRAEWHGGNRDYTQLWKPISVEEIKASRFDWRTSPGPDGIRSGQWRAVPVHLKAEMFNAWMARGEIPEILRQCRTVFVPKVERPGGPGEYRPISIASIPLRHFHSILARRLLACCPPDARQRGFICADGTLENSAVLDAVLGDSRKKLRECHVAVLDFAKAFDTVSHEALVELLRLRGMPEQFCGYIAHLYDTASTTLAVNNEMSSPVKVGRGVRQGDPLSPILFNVVMDLILASLPERVGYRLEMELVSALAYADDLVLLAGSKVGMQESISAVDCVGRQMGLRLNCRKSAVLSMIPDGHRKKHHYLTERTFNIGGKPLRQVSCVERWRYLGVDF

SEQ ID NO: 10 - protein, 803 aa, Artificial Sequence
  SEQ ID NO 10 - R2 RNA BD + RT domain + Endonuclease domain
RAEYARVQELYKKCRSRAAAEVIDGACGGVGHSLEEMETYWRPILERVSDAPGPTPEALHALGRAEWHGGNRDYTQLWKPISVEEIKASRFDWRTSPGPDGIRSGQWRAVPVHLKAEMFNAWMARGEIPEILRQCRTVFVPKVERPGGPGEYRPISIASIPLRHFHSILARRLLACCPPDARQRGFICADGTLENSAVLDAVLGDSRKKLRECHVAVLDFAKAFDTVSHEALVELLRLRGMPEQFCGYIAHLYDTASTTLAVNNEMSSPVKVGRGVRQGDPLSPILFNVVMDLILASLPERVGYRLEMELVSALAYADDLVLLAGSKVGMQESISAVDCVGRQMGLRLNCRKSAVLSMIPDGHRKKHHYLTERTFNIGGKPLRQVSCVERWRYLGVDFEASGCVTLEHSISSALNNISRAPLKPQQRLEILRAHLIPRFQHGFVLGNISDDRLRMLDVQIRKAVGQWLRLPADVPKAYYHAAVQDGGLAIPSVRATIPDLIVRRFGGLDSSPWSVARAAAKSDKIRKKLRWAWKQLRRFSRVDSTTQRPSVRLFWREHLHASVDGRELRESTRTPTSTKWIRERCAQITGRDFVQFVHTHINALPSRIRGSRGRRGGGESSLTCRAGCKVRETTAHILQQCHRTHGGRILRHNKIVSFVAKAMEENKWTVELEPRLRTSVGLRKPDIIASRDGVGVIVDVQVVSGQRSLDELHREKRNKYGNHGELVELVAGRLGLPKAECVRATSCTISWRGVWSLTSYKELRSIIGLREPTLQIVPILALRGSHMNWTRFNQMTSVMGGGV

SEQ ID NO: 11 - DNA, 20 bp, Artificial Sequence
  SEQ ID NO 11 - Forward Primer 5431
tcgggttgctctcatccctg

SEQ ID NO: 12 - DNA, 23 bp, Artificial Sequence
  SEQ ID NO 12 - Reverse Primer 5222
cctctcatgtctcttcaccgtgc

SEQ ID NO: 13 - DNA, 5363 bp, Artificial Sequence
  SEQ ID NO 13 - pcDNA3 backbone sequence
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 14 - DNA, 36 bp, Artificial Sequence
  SEQ ID NO 14 - Kozak-NLS sequence
gccaccatgcctaagaagaagagaaaggtgggtacc

SEQ ID NO: 15 - DNA, 3828 bp, Artificial Sequence
  SEQ ID NO 15 - R2OI protein sequence ORF, codon optimized
atgggcaccgacacagtgtacgtcggccaggattatcctagcggcctgagcaaaagagtgcccgctagactggttgctggccccatgctgagagagagatcttgtcacgcccacgtgttcagagccggacacatgtggaattggagaaccagcctgcctagcggcagatgggatcagcctgctctggaaaagtcccgggtgctgaccagatctgtggccaccgctacagaccccgagatcacatcttaccctggcaagagcgtgtccaccagcacacaggtgcaagaagaggactggtgtagcagagagagcggctggatttctcctggactggcccctgaggaacctagcgtggtgtctgagatcacagcctccatggtggccactatgagagtggctacagaggaagtggtgctggaacctcagcctgagcaggtcgtgacaattctgcccgagcacggcagaaatgtgccaccaggactggccgagcaggataccgcctctcctattgaagtgtccgtgctgctgcccgacctggccgaaaattgtcctctgtgtggtgttcccagcggcggactgagactgctgggaaagcactttgccgttagacatgccggcgtgcccgtgacctacgagtgtagaaagtgtgcctggcggagccccaatagccacagcatctcttgccacgtgccaaagtgcagaggcagagccagaatgccaagcggagatcctggaatcgcctgcgatctgtgcgaggccagatttgccacagaagtgggagtcgcccagcacaagagacacgtgcaccccgtggaatggaacaaagtgcggctggaaagaagaggcgccagaggcggaggaatcaaggccacaaaactttggagcgtggccgaggtggaaaccctgatcagactgattagagagcacggcgatagcggcgccacataccagctgattgccgatgaactcggcagaggcaagacagccgagcaagtgcggagcaagaagcggctgctgagaatcgataccgccagcaactctcccgacgacgccgaagtggaagaggaaagactggaatctctggccgtgcggtccagcagcagatctcctcctagtctggtggctaccagagtgcgggaagctgtggcaaggggagaatctgaaggcggcgaggaaatcagagccattgccgcactgatcagagatgtggatcagaacccctgcctgatcgagacaagcgccagcgacatcatcagcaagctgggcagaagagtggacggccctaaaagacccagacctgtcgtgcgggaacagacccaagaaaaaggctgggtccgacggctggccagacggaagagagagtatagagaggcccagtacctgtacagcagagatcaggcaagactggccgctcagattctggatggcgctgcctctcaagaatgcgccctgcctgtggatcaagtgtacggcgccttccgggaaaagtgggagacagtgggacagtttcacggcctgggcgagtttagaacaggcgctagagccgacaactgggagttctactctcccatcctggctgccgaagtcaaagaaaacctgatgcggatggccaacggcacagcccctggacctgatagaatcagcaagaaggccctgctggactgggaccctagaggcgaacagctggctagactgtacaccacatggctgatcggcggcgtgatccccagagtgttcaaagagtgtcggaccaagctgctgcctaagagcagcgatcctgtggaactgcaggatatcggaggatggcggcctgtgacaatcggcagcatggtcaccagactgttcagcagaatcctgaccatgcggctgacccgggcctgtcctatcaatcctagacagagaggcttcctggccagcagctctggatgtgccgagaacctgctgatcttcgacgagatcgtgcggcggtctagaagagatggtggaccactggccgtggtgttcgtggatttcgccagagccttcgacagcatcagccacgagcacatcctgtgtgttctggaagaaggcggcctggatagacacgtgatcggcctgattcggaacagctacgtggactgtgtgaccagagtgggctgcgtggaaggcatgacacctccaatccagatgaaggtcggagtgaagcagggcgaccctatgagccctctgctgttcaatctggctatggaccctctgattcacaagctggaaacagccggcacaggcctgaagtggggagatctgtctatcgccacactggccttcgccgatgatctggtgctggtgtcagacagcgaagaaggcatgggcagatccctgggcatcctggaaaaattctgccagctgaccggcctgagagtgcagcctagaaagtgccacggcttcttcatggacaagggcgtcgtgaatggctgcggcacatgggagatttgtggcagccctatccacatgatcccaccaggcgaatctgtgcgctatctgggcgttcaagttggccctggaagaggcgtgatggaacccgatctgatccctaccgtgcacacctggatcgagagaatctctgaggcccctctgaagcccagccagagaatgagagtgctgaatagcttcgccctgccacggatcatctatcaggctgacctgggcaaagtgaccgtgacaaagctggcccagatcgatggaattgtgcggaaagccgtgaagaagtggctgcatctgagccccagcacctgtaatggcctgctgtactccagaaacagagatggcggactggggctcctgaagctggaacgactgattcctagcgtgcggaccaagagaatctaccggatgagcagaagccccgacatctggaccagaagaatgaccagccactccgtgtccaagagcgactgggaaatgctgtgggtgcaagctggcggagaaagaggctctgctcctgttatgggagccgtggaagccgctcctaccgatgtggaaagatcccctgactaccccgattggcggagagaggaaaatcttgcttggagcgccctgagagttcaaggcgtgggagctgatcagttcagaggcgatagaacctccagcagctggatcgccgaacctgcctctgtgggatttgcccagagacattggctggctgctctggcacttagagccggcgtgtaccctaccagagagtttctggccaggggcaaagaaaagagcggagccgcctgtagaagatgccctgccagactggaaagctgcagccacatcctgggccagtgtcctttcgtgcaggccaacagaatcgcccggcacaacaaagtgtgcgtgctcctggcaaccgaggccgagagatttggctggaccgtgatccgggaattccggcttgaagatgctgctggcgggctgaagattcccgacctcgtgtgtaaaaaggccgacaccgtgctgatcgtggacgtgaccgtcagatacgagatggacggcgagacactgaagagagccgccagcgagaaagtgaagcactatctgccagtgggccagcagatcaccgacaaagtcggcggacggtgcttcaaagtgatgggctttcctgtgggcgcaagaggcaaatggccagcctctaacaataccgtgctggccgaacttggagtgccagccggcagaatgaggacctttgctaggctggtgtcccggcggacactgctgtatagcctggacatcctgcgggacttcatgagagagcctgccggaagaggtacaagagtggcactgattccagctgccacaggcgctgctaac

SEQ ID NO: 16 - DNA, 135 bp, Artificial Sequence
  SEQ ID NO 16 - HA-NLS-P2A sequence
ggatcctacccatacgatgttccagattacgcggccgctccaaaaaagaaaagaaaagttgaattcggcggcagcggcgccaccaacttcagcctgctgaagcaggccggcgacgtggaggagaaccccggcccc

SEQ ID NO: 17 - DNA, 711 bp, Artificial Sequence
  SEQ ID NO 17 - mCherry sequence
atggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtag

SEQ ID NO: 18 - DNA, 3342 bp, Artificial Sequence
  SEQ ID NO 18 - R2 protein sequence ORF, codon optimized
atgatggccagcacagccctgtctctgatgggcagatgcaatcccgatggctgcacaagaggcaagcacgtgacagccgctcctatggatggacctagaggaccttctagcctggccggcacatttggatggggacttgctattcctgccggcgagccttgtggcagagtgtgttctcctgccaccgtgggattcttcccagtggccaagaagtccaacaaagagaacagacccgaggccagcggcctgcctctggaatctgaaagaaccggcgataatcctaccgtgcggggatctgctggtgccgatcctgttggacaagatgcccctggctggacctgccagttctgcgagagaaccttcagcaccaatagaggcctgggcgtgcacaaaagacgggctcaccctgtggaaacaaacaccgacgctgcccctatgatggtcaagagaagatggcacggcgaggaaatcgacctgctggccagaacagaagccagactgctggctgagaggggacagtgttctggcggagatctgtttggcgccctgcctggctttggaagaaccctggaagccatcaagggccagcgcagaagagagccttatagagccctggtgcaggcccacctggccagatttggatctcagcctggacctagctctggcggatgtagcgccgaacctgattttcggagagcctctggcgctgaagaggccggcgaagaaagatgtgctgaggatgccgccgcttacgatccttctgctgtgggccaaatgagccctgatgccgccagagtgctgtctgaacttcttgaaggcgctggcagacgcagagcctgtagagccatgaggcctaagaccgccggaagaagaaacgacctgcacgacgatagaaccgccagcgctcacaagaccagcagacagaagagaagggccgagtacgccagggtgcaagagctgtacaagaagtgcagatccagagccgccgctgaagtgattgatggtgcttgtggtggcgtgggccacagcctggaagagatggaaacctattggcggcccatcctggaaagagtgtctgacgctcctggaccaacacctgaagctctgcatgctctgggcagagctgagtggcatggcggcaatagagattacacccagctgtggaagcccatcagcgtggaagaaatcaaggccagcagattcgactggcggacaagccctggacctgatggcattagatctggacagtggcgggctgtgcctgtgcacctgaaggccgaaatgttcaacgcctggatggccagaggcgagatccctgagatcctgagacagtgcagaaccgtgttcgtgcccaaggtggaaagacctggcggaccaggcgagtacagacccatctctatcgccagcattcctctgcggcacttccactctatcctggctcggagacttctggcctgctgtcctcctgatgccagacagagaggctttatctgcgccgacggcaccctggaaaattctgcagtgctggatgccgtgctgggcgactctcggaagaaactgagagaatgtcacgtggccgtcctggacttcgccaaggcctttgatacagtgtctcacgaggccctggtggaactgctgagactgaggggaatgcctgagcagttctgtggctatatcgcccacctgtacgacaccgcctctaccacactggccgtgaacaatgagatgagcagccccgtgaaagttggcagaggcgttagacagggcgaccctctgagccccatcctgttcaatgtggtcatggatctgatcctggccagcctgcctgagagagtgggctatagactggaaatggaactggtgtctgccctggcctacgccgatgatctggttctgcttgccggcagcaaagtgggcatgcaagagtctatcagcgccgtggattgcgtgggcagacagatgggcctgcgcctgaattgcagaaaaagcgccgtgctgagcatgatccccgatggccacagaaagaagcaccactacctgaccgagcggaccttcaatatcggcggcaagcctctgagacaggtgtcctgtgttgagagatggcggtatctgggcgtcgactttgaggcctctggctgtgtgacactggaacactctatcagcagcgccctgaacaacatcagcagagcccctctgaagcctcagcagcggctggaaattctgagagcccatctgatccctcggttccagcacggatttgtgctgggcaacatctccgacgaccggctgagaatgctggacgtgcagatcagaaaagccgtcggccagtggctgagacttcctgcagatgtgcctaaggcctactatcacgctgctgtgcaagatggcggcctggctattccttctgtgcgcgccacaattcccgacctgatcgtgcgaagattcggcggacttgatagctctccttggagcgtggccagagctgccgccaagagcgataagatccggaaaaagctgcgctgggcctggaagcagctgcggagattttctagagtggacagcaccacacagcggcctagtgtgcggctgttttggagagaacatctgcacgcctccgtggacggcagagagctgagagaaagcaccagaacacccaccagcaccaagtggatcagagagagatgcgcccagatcacaggccgggatttcgtgcagttcgtgcacacccatatcaacgccctgccatccagaatcaggggcagcagaggtagaagaggcggaggcgaaagcagcctgacatgtagagccggctgtaaagtgcgcgagacaacagcccacatcctgcagcagtgtcatagaacacacggcggcagaatcctgcggcacaacaagattgtgtccttcgtggccaaggccatggaagagaacaagtggaccgtggaactggaacccagactgagaacaagcgtgggcctgagaaagcccgacatcattgcctctcgagatggcgtgggagtgatcgtggatgtgcaggttgtgtcaggccagagaagcctggacgagctgcacagagagaagcggaacaaatacggcaaccacggcgagctggttgaactggttgcaggcagactgggactgccaaaagccgagtgtgtgcgggccacctcttgtaccatttcttggagaggcgtgtggtccctgaccagctacaaagagctgcggtccatcatcggactgagagagcctacactgcagatcgtccccattctggccctgagaggcagccacatgaattggacccgcttcaaccagatgaccagcgtgatgggaggcggcgttgga

SEQ ID NO: 19 - DNA, 2221 bp, Artificial Sequence
  SEQ ID NO 19 - pTwist vector backbone
aggctaggtggaggctcagtgatgataagtctgcgatggtggatgcatgtgtcatggtcatagctgtttcctgtgtgaaattgttatccgctcagagggcacaatcctattccgcgctatccgacaatctccaagacattaggtggagttcagttcggcgtatggcatatgtcgctggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctctattcaacaaagccgccgtcccgtcaagtcagcgtaaatgggtagggggcttcaaatcgtcctcgtgataccaattcggagcctgcttttttgtacaaacttgttgataatggcaattcaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagagccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccagatacctgaaacaaaacccatcgtacggccaaggaagtctccaataactgtgatccaccacaagcgccagggttttcccagtcacgacgttgtaaaacgacggccagtcatgcataatccgcacgcatctggaataaggaagtgccattccgcctgacct

SEQ ID NO: 20 - DNA, 222 bp, Artificial Sequence
  SEQ ID NO 20 - polI - RNA polymerase I promoter
gccgggagggcgtccccggcccggcgctgctcccgcgtgtgtcctggggttgaccagagggccccgggcgctccgtgtgtggctgcgatggtggcgtttttggggacaggtgtccgtgtcgcgcgtcgcctgggccggcggcgtggtcggtgacgcgacctcccggccccgggggaggtatatctttcgctccgagtcggcattttgggccgccgggttatt

SEQ ID NO: 21 - DNA, 265 bp, Artificial Sequence
  SEQ ID NO 21 - R2OI 5′UTR
cgcacaggggacacagagcctgcccaagtaccgctcccgagggagcgggaaacgggggggtgactatcccctggggtccggcgagagcgctggtctacggaccaggggtggctgtgggcaggctgctcctcaggccagttgattagttacgcatgggctgtacctccacgtggtcccgctggtaacgacttgtcggctaaatcagcccgcccaccatctgggatatggttgaccgtctaaccccagtactcaggtcacaaacaaa

SEQ ID NO: 22 - DNA, 3831 bp, Artificial Sequence
  SEQ ID NO 22 - R2OI for RNA R2OI ORF
atgggaacagatacagtgtatgtcggccaggactacccttctggcttatcaaaacgggtaccagcacggttagtggcgggaccgatgctgcgagagcgaagctgtcacgcccatgtgtttagggctggacacatgtggaactggcgaaccagccttccgagcgggcgctgggaccagcccgctttggagaagtctcgggtcctaacccggtcggtggcgacggccaccgaccccgaaattacctcttacccaggaaagtccgtatcgacaagtacgcaggttcaggaggaggactggtgtagccgggagagcgggtggatctcgccaggacttgctcctgaagaaccctcggtggtgtccgaaattacagcctccatggtagcgacaatgagggtagcaaccgaggaggtcgtgctggaaccacagcctgaacaggtcgtcacaatactgccggagcatggtcgaaacgttcctccggggctggcagaacaggacaccgccagccccatagaagtctcggtgctcctcccagacctcgctgagaactgcccattgtgtggcgtgccgagcgggggcctacgcttgctcgggaagcattttgctgtccgacatgcgggggtgcctgtaacgtatgagtgccgtaagtgtgcgtggcggagccccaacagccactcaatctcgtgtcacgtccccaaatgccgggggcgtgcgcggatgcccagtggcgatccagggatcgcctgcgatctctgtgaagcccggtttgccacggaggttggggtcgcccaacacaagcggcacgttcatccggtggagtggaacaaggtgaggctggaaaggagaggtgcgcgcggagggggaattaaggcgacgaagctctggagtgtagcggaggtagagacgctaatccggctcatccgtgagcacggagattcaggtgccacttaccagctcattgccgatgagctgggaaggggcaagacggccgaacaggtgaggagtaaaaagaggctcctgcgcatagatacggcaagcaatagcccagatgatgcagaggttgaggaggagaggttggaatctctggcggttcggtcctcgtcacggtcacccccgagcctggtggcgaccagggtcagggaggcagttgccaggggtgaatcagaaggtggcgaggagatcagggctattgctgctctcattagggacgtagatcagaatccttgtctgattgaaacctcggcgtcggacatcatctcgaagctgggaaggagggtggatgggcccaagagacccaggcccgttgtcagagaacagacccaagagaagggatgggtaaggcggcttgcccggcggaaaagggagtacagagaagcgcagtacctgtactcaagggatcaagcaaggctggcggcccagatcctcgatggtgccgccagccaggaatgcgccctcccggtggaccaggtctacggagcgttccgtgagaaatgggaaaccgtagggcagttccacggacttggtgagttccggacgggtgcacgcgcagacaactgggagttctactctccaattctggcggctgaggtgaaagaaaacctaatgagaatggctaacggcacggccccgggaccagacaggataagcaaaaaggctctgcttgactgggacccccggggtgagcaactggcacggctgtacacgacgtggctgatcggtggggtcataccaagggtcttcaaggagtgcaggactaagctgctaccgaaatccagcgacccggtcgagttgcaggacatcggtggatggaggccggtgacgattgggtcgatggtgactaggctgttcagtcggattctaacgatgaggctaacccgagcctgtccgatcaatccgaggcagcgcggtttcttggcctcctcgagtggatgcgcggaaaacctgttgatctttgacgagatcgtcaggcgctcgaggcgggacggggggccgctggcagtggtgtttgtggactttgcgagggcctttgactccatctcacatgaacatatcctgtgtgttctcgaagaaggcgggcttgacaggcacgttatcgggttgatccgaaactcgtacgtggattgcgtgaccagggtgggttgtgtcgagggcatgacaccaccaatacaaatgaaggttggagtgaagcagggagaccccatgtcccccttgctcttcaacctggctatggatcccctcatccataaactcgagacggccggaactggactgaaatggggcgatctttcaatcgccacgctggcctttgccgacgatctggtgctggtgagtgactctgaggaaggcatggggaggagtctcgggattttggagaagttttgccaactgactgggctgagggttcagcccaggaagtgtcacggtttctttatggacaagggcgtggtgaacggctgtggaacctgggaaatctgtgggtcaccgatccacatgattcccccgggggaatcagttcgttatttgggagtccaggtaggcccggggcgcggcgtgatggaaccggatcttatccctacggtccacacgtggatcgaaaggatctcggaggctcctctaaagccctcacaacgcatgagggttttgaactcattcgctctcccccggataatttaccaggccgatctagggaaggttacggtaaccaaattggcccagatagatgggattgtccggaaggctgtgaagaagtggctccatttgtcaccatccacgtgcaatggactgctgtattcacggaaccgcgacggtggtttgggcctcctaaagctggaaagactaatcccatccgtgcgcacgaagcgtatctatcggatgtccaggtctccggatatctggacacggcgaatgaccagccattctgtgtcaaaatctgactgggagatgttgtgggtccaagcgggaggtgagaggggcagtgcacctgtaatgggtgccgtggaggctgccccgaccgatgtggagagatcgccagactacccagactggcggcgtgaggaaaacctggcatggtcggccctgcgggtgcagggtgtgggtgcagaccagtttcgaggcgacaggaccagcagctcttggatcgccgagcccgcttcggttgggttcgcgcagcgccactggttggctgccctggcgctgagggctggggtgtatccgactcgggagtttctggctcggggtaaggaaaagtcaggagcagcttgcagacgctgcccggccaggttggaatcatgttcacacatacttgggcaatgtccgttcgttcaggcgaacagaattgcgaggcacaacaaggtgtgtgtgctcttggccacggaggcggagaggttcggctggacggtaataagggagttccgtcttgaggacgccgctggcggtctcaagatacccgacctggtttgcaagaaggccgacacagttctcattgtcgacgtgaccgtccggtacgagatggatggagagacgctaaaaagggccgcatcggagaaggtgaaacactatctcccagtagggcaacagataacggacaaggtcggagggcgttgctttaaagtcatggggttccctgtaggtgctaggggaaagtggccggcgagcaacaacacagttttggctgagttaggcgtccctgcaggtcggatgaggacctttgccaggctggtgagccggaggactcttctttattctttggatatattgagggacttcatgcgtgagccggccggcaggggaactcgggttgctctcatccctgcggcaacgggtgccgcgaattga

SEQ ID NO: 23 - DNA, 108 bp, Artificial Sequence
  SEQ ID NO 23 - R2OI 3′UTR
gggggacagctgggagtctcggcatgattacaaatcttgcgctgcactcggatgtcgtccccgtgacggacacattaatccggaaagcgagtggtgactcgcctcaag

SEQ ID NO: 24 - DNA, 3345 bp, Artificial Sequence
  SEQ ID NO 24 - R2 for RNA ORF
atgatggcgagcaccgcactgtcccttatgggacggtgtaacccggatggctgtacacgtggtaaacacgtgacagcagccccgatggacggaccgcgaggaccgtcaagcctagcaggtaccttcgggtggggccttgcgatacctgcgggcgaaccctgtggtcgggtttgcagcccggccacagtgggtttttttcctgttgcaaaaaagtcaaataaagaaaatagacctgaagcctctggcctcccgctggagtcagagaggacaggcgataacccgactgtgcggggttccgccggcgcagatcctgtgggtcaggatgcgcctggttggacctgccagttctgcgaacgaaccttttcgaccaacaggggtttgggtgtccacaagcgtagagcccaccctgttgagaccaatacggatgccgctccgatgatggtgaagcggcggtggcatggcgaggaaatcgacctcctcgctcgcaccgaggccaggttgctcgctgagcggggtcagtgctcgggtggagacctctttggcgcgcttccagggtttggaagaactctggaagcgattaagggacaacggcggagggagccttatcgggcattggtgcaagcgcaccttgcccgatttggttcccagccgggtccctcgtcgggggggtgctcggccgagcctgacttccggcgggcttctggagctgaggaagcgggcgaggaacgatgcgccgaagacgccgctgcctatgatccatccgcagtcggtcagatgtcgcccgatgccgctcgggttctctccgaactccttgagggtgcggggagaagacgagcgtgcagggctatgagacccaagactgcagggcggcgaaacgatttgcacgatgatcggacagctagtgcccacaaaaccagtagacaaaagcgcagggcagagtacgcgcgtgtgcaggaactgtacaagaagtgtcgcagcagagcagcagctgaggtgatcgatggcgcgtgtgggggtgtcggacactcgctcgaggagatggagacctattggcgacctatcctcgagagagtgtccgatgcacctgggcctacaccggaagctcttcacgccctagggcgtgcggagtggcacgggggcaatcgcgactacacccagctgtggaagccgatctcggtggaagagatcaaggcctcccgctttgactggcgaacttcgccgggcccggacggtatacgttcgggtcagtggcgtgcggttcctgtgcacttgaaggcggaaatgttcaatgcatggatggcacgaggcgaaatacccgaaattctacggcagtgccgaaccgtctttgtacctaaggtggagagaccaggtggaccgggggaatatcgaccgatctcgatcgcgtcgattcccctgagacactttcactccatcttggcccggaggctgttggcttgctgcccccctgatgcacgacagcgcggatttatctgcgccgacggtacgctggagaattccgcagtactggacgcggtgcttggggatagcaggaagaagctgcgggaatgtcacgtggcggtgctagacttcgccaaggcatttgacacagtgtctcacgaggcacttgtcgaattgctgaggttgaggggcatgcccgaacagttctgcggctacattgctcacctatacgatacggcgtccaccaccttagccgtgaacaatgaaatgagcagccctgtaaaagtgggacgaggggttcgtcaaggggaccctctgtcgccgatactcttcaacgtggtgatggacctcatcctggcttccctgccggagagggtcgggtataggttggagatggaactcgtgtccgctctggcctatgctgacgacctagtcctgcttgcggggtcgaaggtagggatgcaggagtccatctctgctgtggactgtgtcggtaggcagatgggcctacgcctgaattgcaggaaaagcgcggttctgtctatgataccggatggccaccgcaagaagcatcactacctgactgagcgaaccttcaatattggaggtaagccgctcaggcaggtgagttgtgttgagcggtggcgatatcttggtgtcgattttgaggcctctggatgcgtgacattagagcatagtatcagtagtgctctgaataacatctcaagggcacctctcaaaccccaacagaggttggagattttgagagctcatctgattccgagattccagcacggttttgtgcttggaaacatctcggatgaccgattgagaatgctcgatgtccaaatccggaaagcagtcggacagtggctaaggctaccggcggatgtgcccaaggcatattatcacgccgcagttcaggacggcggcttagcgatcccatcggtgcgagcgaccatcccggacctcattgtgaggcgtttcggggggctcgactcgtcaccatggtcagtggcaagagccgccgccaaatctgataagattcgtaagaaactgcggtgggcctggaaacagctccgcaggttcagccgtgttgactccacaacgcaacgaccatctgtgcgcttgttttggcgagaacatctgcatgcatctgttgatggacgcgaacttcgcgaatccacacgcaccccgacatccacaaagtggattagggagcgatgcgcgcagataaccggacgggacttcgtgcagttcgtgcacactcatatcaacgccctcccatcccgcattcgcggatcgagagggcgtagaggtgggggtgagtcttcgttgacctgccgtgctggttgcaaggttagggagacgacggctcacatcctacaacagtgtcacagaacacacggcggccggattctacgacacaacaagattgtatctttcgtggcgaaagccatggaagagaacaagtggacggttgagctggagccgaggctacgaacatcggttggtctccgtaagccggatattatcgcctccagggatggtgtcggagtgatcgtggacgtgcaggtggtctcgggccagcgatcgcttgacgagctccaccgtgagaaacgtaataaatacgggaatcacggggagctggttgagttggtcgcaggtagactaggacttccgaaagctgagtgcgtgcgagccacttcgtgcacgatatcttggaggggagtatggagcctgacttcttataaggagttaaggtccataatcgggcttcgggaaccgacactacaaatcgttccgatactggcgttgagaggttcacacatgaactggaccaggttcaatcagatgacgtccgtcatggggggcggcgttggttga

SEQ ID NO: 25 - DNA, 620 bp, Artificial Sequence
  SEQ ID NO 25 - R2 5′UTR
gcgggagtaactatgactctcttaggggcgatacgcataattttaatttttcgattcaaatccagtcgtcttaatctggtgaccagtggcgcggtcaccagtatagtgcacaggacgtgaatggctccgaggctggcggagtcactcactataagtgtgagagacgatgtcctgtgccaagtatacgtccaaccctaacgggttaagtgaaattagttgctcataacagggacggtgtacctgtttgctcgtggctggctatcgaatggacgggaccaatacacccccctgttagtaatggggtaagagagagcggtctgaaactatggccgagatcacgacgccccactcctacccataacctgcacgtggtaccgccgcacattgaccgatacgggaggaggggcagcacttgaatcacgtagtcttggtgtagccattgcgggactacagccctcgtaagtgccgccttagaacgcaacggggcaataggtgggccggggcgctagcgggggggagtaatctcccctgttggcgtgcaccgcactgctccctctgggggcagtgtcatccggaaacaggtgggccggggcgccaccaggggggagcaatccctcctg

SEQ ID NO: 26 - DNA, 248 bp, Artificial Sequence; identical to SEQ ID NO: 8
  SEQ ID NO 26 - R2 3′UTR

SEQ ID NO: 27 - DNA, 106 bp, Artificial Sequence
  SEQ ID NO 27 - r106 homology arm - 106 bp sequence 28S human ribosomal gene upstream from the insertion site
gcgggtgttgacgcgatgtgatttctgcccagtgctctgaatgtcaaagtgaagaaattcaatgaagcgcgggtaaacggcgggagtaactatgactctcttaagg

SEQ ID NO: 28 - DNA, 100 bp, Artificial Sequence
  SEQ ID NO 28 - r100 homology arm - 100bp sequence of 28S human ribosomal gene downstream from the insertion site
tagccaaatgcctcgtcatctaattagtgacgcgcatgaatggatgaacgagattcccactgtccctacctactatccagcgaaaccacagccaagggaa

SEQ ID NO: 29 - DNA, 30 bp, Artificial Sequence
  SEQ ID NO 29 - r30 homology arm - 30bp sequence of 28S human ribosomal gene downstream from the insertion site
tagccaaatgcctcgtcatctaattagtga

SEQ ID NO: 30 - DNA, 15 bp, Artificial Sequence
  SEQ ID NO 30 - r15 homology arm - 15bp sequence of 28S human ribosomal gene downstream from the insertion site
tagccaaatgcctcg

SEQ ID NO: 31 - DNA, 10 bp, Artificial Sequence
  SEQ ID NO 31 - r10 homology arm - 10bp sequence of 28S human ribosomal gene downstream from the insertion site
tagccaaatg

SEQ ID NO: 32 - DNA, 35 bp, Artificial Sequence
  SEQ ID NO 32 - polI terminator - 35bp, RNA polymerase I terminator
tcccccccaacttcggaggtcgaccagtactccgg

SEQ ID NO: 33 - DNA, 58 bp, Artificial Sequence
  SEQ ID NO 33 - NGS Forward Primer
  misc_feature (34)..(36): n is a, c, g, or t
tcgtcggcagcgtcagatgtgtataagagacagnnngtagcctcagtcttcccatcag

SEQ ID NO: 34 - DNA, 55 bp, Artificial Sequence
  SEQ ID NO 34 - NGS Reverse Primer
  misc_feature (35)..(37): n is a, c, g, or t
gtctcgtgggctcggagatgtgtataagagacagnnncagcagcaagcagcactc

SEQ ID NO: 35 - DNA, 20 bp, Artificial Sequence
  SEQ ID NO 35 - EMX guide RNA sequence
gagtccgagcagaagaagaa

SEQ ID NO: 36 - DNA, 123 bp, Artificial Sequence
  SEQ ID NO 36 - HA-NLS-XTEN sequence
ggatcctacccatacgatgttccagattacgcggccgctccaaaaaagaaaagaaaagttgaattcggcggcagcagcggcagcgagactcccgggacctcagagtccgccacacccgaaagt

SEQ ID NO: 37 - DNA, 48 bp, Artificial Sequence
  SEQ ID NO 37 - XTEN linker sequence
agcggcagcgagactcccgggacctcagagtccgccacacccgaaagt

SEQ ID NO: 38 - DNA, 171 bp, Artificial Sequence
  SEQ ID NO 38 - HA-NLS-32aa sequence
ggatcctacccatacgatgttccagattacgcggccgctccaaaaaagaaaagaaaagttgaattcggcggcagctctggtggttcttctggtggttctagcggcagcgagactcccgggacctcagagtccgccacacccgaaagttctggtggttcttctggtggttct

SEQ ID NO: 39 - DNA, 96 bp, Artificial Sequence
  SEQ ID NO 39 - 32aa linker sequence
tctggtggttcttctggtggttctagcggcagcgagactcccgggacctcagagtccgccacacccgaaagttctggtggttcttctggtggttct

SEQ ID NO: 40 - DNA, 4107 bp, Artificial Sequence
  SEQ ID NO 40 - SpCas9 Human codon optimized
atggacaagaagtatagcatcggcctggatatcggcacaaactccgtgggctgggccgtgatcaccgacgagtacaaggtgccaagcaagaagtttaaggtgctgggcaacaccgatagacactccatcaagaagaatctgatcggcgccctgctgttcgactctggcgagacagccgaggccacacggctgaagagaaccgcccggagaaggtatacacgccggaagaataggatctgctacctgcaggagatcttcagcaacgagatggccaaggtggacgattctttctttcaccgcctggaggagagcttcctggtggaggaggataagaagcacgagcggcaccctatctttggcaacatcgtggacgaggtggcctatcacgagaagtacccaacaatctatcacctgaggaagaagctggtggactccaccgataaggccgacctgcgcctgatctatctggccctggcccacatgatcaagttccggggccactttctgatcgagggcgatctgaacccagacaatagcgatgtggacaagctgttcatccagctggtgcagacctacaatcagctgtttgaggagaaccccatcaatgcctctggagtggacgcaaaggcaatcctgagcgccagactgtccaagtctagaaggctggagaacctgatcgcccagctgccaggcgagaagaagaacggcctgtttggcaatctgatcgccctgtccctgggcctgacacccaacttcaagtctaattttgatctggccgaggacgccaagctgcagctgtccaaggacacctatgacgatgacctggataacctgctggcccagatcggcgatcagtacgccgacctgttcctggccgccaagaatctgtctgacgccatcctgctgagcgatatcctgcgcgtgaacaccgagatcacaaaggcccccctgagcgcctccatgatcaagagatatgacgagcaccaccaggatctgaccctgctgaaggccctggtgaggcagcagctgcctgagaagtacaaggagatcttctttgatcagagcaagaatggatacgcaggatatatcgacggaggagcatcccaggaggagttctacaagtttatcaagcctatcctggagaagatggacggcacagaggagctgctggtgaagctgaatcgggaggacctgctgaggaagcagcgcacctttgataacggcagcatccctcaccagatccacctgggagagctgcacgcaatcctgcgccggcaggaggacttctacccatttctgaaggataaccgggagaagatcgagaagatcctgacattcagaatcccctactatgtgggacctctggcccggggcaatagcagatttgcctggatgacccgcaagtccgaggagacaatcacaccctggaacttcgaggaggtggtggataagggcgcctctgcccagagcttcatcgagcggatgaccaattttgacaagaacctgcctaatgagaaggtgctgccaaagcactctctgctgtacgagtatttcaccgtgtataacgagctgacaaaggtgaagtacgtgaccgagggcatgagaaagcctgccttcctgagcggcgagcagaagaaggccatcgtggacctgctgtttaagaccaataggaaggtgacagtgaagcagctgaaggaggactatttcaagaagatcgagtgttttgattctgtggagatcagcggcgtggaggacaggtttaacgcctccctgggcacctaccacgatctgctgaagatcatcaaggataaggacttcctggacaacgaggagaatgaggatatcctggaggacatcgtgctgaccctgacactgtttgaggatagggagatgatcgaggagcgcctgaagacatatgcccacctgttcgatgacaaagtgatgaagcagctgaagagaaggcgctacaccggatggggccggctgagcagaaagctgatcaatggcatccgcgacaagcagtctggcaagacaatcctggactttctgaagagcgatggcttcgccaaccggaacttcatgcagctgatccacgatgactccctgaccttcaaggaggatatccagaaggcacaggtgtctggacagggcgacagcctgcacgagcacatcgccaacctggccggctctcctgccatcaagaagggcatcctgcagaccgtgaaggtggtggacgagctggtgaaagtgatgggcaggcacaagccagagaacatcgtgatcgagatggcccgcgagaatcagaccacacagaagggccagaagaactcccgggagagaatgaagagaatcgaggagggcatcaaggagctgggctctcagatcctgaaggagcaccccgtggagaacacacagctgcagaatgagaagctgtatctgtactatctgcagaatggccgggatatgtacgtggaccaggagctggatatcaacagactgtctgattatgacgtggatcacatcgtgccacagtccttcctgaaggatgactctatcgacaataaggtgctgaccaggagcgacaagaaccgcggcaagtccgataatgtgccctctgaggaggtggtgaagaagatgaagaactactggaggcagctgctgaatgccaagctgatcacacagaggaagtttgataacctgaccaaggcagagaggggaggactgtccgagctggacaaggccggcttcatcaagcggcagctggtggagacaagacagatcacaaagcacgtggcccagatcctggattctagaatgaacacaaagtacgatgagaatgacaagctgatcagggaggtgaaagtgatcaccctgaagtccaagctggtgtctgactttaggaaggatttccagttttataaggtgcgcgagatcaacaattatcaccacgcccacgacgcctacctgaacgccgtggtgggcacagccctgatcaagaagtaccctaagctggagtccgagttcgtgtacggcgactataaggtgtacgatgtgcgcaagatgatcgccaagtctgagcaggagatcggcaaggccaccgccaagtatttcttttacagcaacatcatgaatttctttaagaccgagatcacactggccaatggcgagatcaggaagcgcccactgatcgagacaaacggcgagacaggcgagatcgtgtgggacaagggcagggattttgccaccgtgcgcaaggtgctgagcatgccccaagtgaatatcgtgaagaagaccgaggtgcagacaggcggcttctccaaggagtctatcctgcctaagcggaactccgataagctgatcgccagaaagaaggactgggaccccaagaagtatggcggcttcgacagccctacagtggcctactccgtgctggtggtggccaaggtggagaagggcaagagcaagaagctgaagtccgtgaaggagctgctgggcatcaccatcatggagcgcagctccttcgagaagaatcctatcgactttctggaggccaagggctataaggaggtgaagaaggacctgatcatcaagctgccaaagtactctctgtttgagctggagaacggaaggaagagaatgctggcaagcgccggagagctgcagaagggcaatgagctggccctgccctccaagtacgtgaacttcctgtatctggcctcccactacgagaagctgaagggctctcctgaggataacgagcagaagcagctgtttgtggagcagcacaagcactatctggacgagatcatcgagcagatcagcgagttctccaagagagtgatcctggccgacgccaatctggataaggtgctgtccgcctacaacaagcaccgggataagccaatcagagagcaggccgagaatatcatccacctgtttaccctgacaaacctgggagcaccagcagccttcaagtattttgacaccacaatcgacaggaagcggtacaccagcacaaaggaggtgctggacgccacactgatccaccagtccatcaccggcctgtacgagacacggatcgacctgtctcagctgggaggcgattga

SEQ ID NO: 41 - DNA, 4107 bp, Artificial Sequence
  SEQ ID NO 41 - Dead_SpCas9 D10A and N863A mutations human codon optimized
atggacaagaagtatagcatcggcctggccatcggcacaaactccgtgggctgggccgtgatcaccgacgagtacaaggtgccaagcaagaagtttaaggtgctgggcaacaccgatagacactccatcaagaagaatctgatcggcgccctgctgttcgactctggcgagacagccgaggccacacggctgaagagaaccgcccggagaaggtatacacgccggaagaataggatctgctacctgcaggagatcttcagcaacgagatggccaaggtggacgattctttctttcaccgcctggaggagagcttcctggtggaggaggataagaagcacgagcggcaccctatctttggcaacatcgtggacgaggtggcctatcacgagaagtacccaacaatctatcacctgaggaagaagctggtggactccaccgataaggccgacctgcgcctgatctatctggccctggcccacatgatcaagttccggggccactttctgatcgagggcgatctgaacccagacaatagcgatgtggacaagctgttcatccagctggtgcagacctacaatcagctgtttgaggagaaccccatcaatgcctctggagtggacgcaaaggcaatcctgagcgccagactgtccaagtctagaaggctggagaacctgatcgcccagctgccaggcgagaagaagaacggcctgtttggcaatctgatcgccctgtccctgggcctgacacccaacttcaagtctaattttgatctggccgaggacgccaagctgcagctgtccaaggacacctatgacgatgacctggataacctgctggcccagatcggcgatcagtacgccgacctgttcctggccgccaagaatctgtctgacgccatcctgctgagcgatatcctgcgcgtgaacaccgagatcacaaaggcccccctgagcgcctccatgatcaagagatatgacgagcaccaccaggatctgaccctgctgaaggccctggtgaggcagcagctgcctgagaagtacaaggagatcttctttgatcagagcaagaatggatacgcaggatatatcgacggaggagcatcccaggaggagttctacaagtttatcaagcctatcctggagaagatggacggcacagaggagctgctggtgaagctgaatcgggaggacctgctgaggaagcagcgcacctttgataacggcagcatccctcaccagatccacctgggagagctgcacgcaatcctgcgccggcaggaggacttctacccatttctgaaggataaccgggagaagatcgagaagatcctgacattcagaatcccctactatgtgggacctctggcccggggcaatagcagatttgcctggatgacccgcaagtccgaggagacaatcacaccctggaacttcgaggaggtggtggataagggcgcctctgcccagagcttcatcgagcggatgaccaattttgacaagaacctgcctaatgagaaggtgctgccaaagcactctctgctgtacgagtatttcaccgtgtataacgagctgacaaaggtgaagtacgtgaccgagggcatgagaaagcctgccttcctgagcggcgagcagaagaaggccatcgtggacctgctgtttaagaccaataggaaggtgacagtgaagcagctgaaggaggactatttcaagaagatcgagtgttttgattctgtggagatcagcggcgtggaggacaggtttaacgcctccctgggcacctaccacgatctgctgaagatcatcaaggataaggacttcctggacaacgaggagaatgaggatatcctggaggacatcgtgctgaccctgacactgtttgaggatagggagatgatcgaggagcgcctgaagacatatgcccacctgttcgatgacaaagtgatgaagcagctgaagagaaggcgctacaccggatggggccggctgagcagaaagctgatcaatggcatccgcgacaagcagtctggcaagacaatcctggactttctgaagagcgatggcttcgccaaccggaacttcatgcagctgatccacgatgactccctgaccttcaaggaggatatccagaaggcacaggtgtctggacagggcgacagcctgcacgagcacatcgccaacctggccggctctcctgccatcaagaagggcatcctgcagaccgtgaaggtggtggacgagctggtgaaagtgatgggcaggcacaagccagagaacatcgtgatcgagatggcccgcgagaatcagaccacacagaagggccagaagaactcccgggagagaatgaagagaatcgaggagggcatcaaggagctgggctctcagatcctgaaggagcaccccgtggagaacacacagctgcagaatgagaagctgtatctgtactatctgcagaatggccgggatatgtacgtggaccaggagctggatatcaacagactgtctgattatgacgtggatcacatcgtgccacagtccttcctgaaggatgactctatcgacaataaggtgctgaccaggagcgacaaggcccgcggcaagtccgataatgtgccctctgaggaggtggtgaagaagatgaagaactactggaggcagctgctgaatgccaagctgatcacacagaggaagtttgataacctgaccaaggcagagaggggaggactgtccgagctggacaaggccggcttcatcaagcggcagctggtggagacaagacagatcacaaagcacgtggcccagatcctggattctagaatgaacacaaagtacgatgagaatgacaagctgatcagggaggtgaaagtgatcaccctgaagtccaagctggtgtctgactttaggaaggatttccagttttataaggtgcgcgagatcaacaattatcaccacgcccacgacgcctacctgaacgccgtggtgggcacagccctgatcaagaagtaccctaagctggagtccgagttcgtgtacggcgactataaggtgtacgatgtgcgcaagatgatcgccaagtctgagcaggagatcggcaaggccaccgccaagtatttcttttacagcaacatcatgaatttctttaagaccgagatcacactggccaatggcgagatcaggaagcgcccactgatcgagacaaacggcgagacaggcgagatcgtgtgggacaagggcagggattttgccaccgtgcgcaaggtgctgagcatgccccaagtgaatatcgtgaagaagaccgaggtgcagacaggcggcttctccaaggagtctatcctgcctaagcggaactccgataagctgatcgccagaaagaaggactgggaccccaagaagtatggcggcttcgacagccctacagtggcctactccgtgctggtggtggccaaggtggagaagggcaagagcaagaagctgaagtccgtgaaggagctgctgggcatcaccatcatggagcgcagctccttcgagaagaatcctatcgactttctggaggccaagggctataaggaggtgaagaaggacctgatcatcaagctgccaaagtactctctgtttgagctggagaacggaaggaagagaatgctggcaagcgccggagagctgcagaagggcaatgagctggccctgccctccaagtacgtgaacttcctgtatctggcctcccactacgagaagctgaagggctctcctgaggataacgagcagaagcagctgtttgtggagcagcacaagcactatctggacgagatcatcgagcagatcagcgagttctccaagagagtgatcctggccgacgccaatctggataaggtgctgtccgcctacaacaagcaccgggataagccaatcagagagcaggccgagaatatcatccacctgtttaccctgacaaacctgggagcaccagcagccttcaagtattttgacaccacaatcgacaggaagcggtacaccagcacaaaggaggtgctggacgccacactgatccaccagtccatcaccggcctgtacgagacacggatcgacctgtctcagctgggaggcgattga

SEQ ID NO: 42 - DNA, 401 bp, Artificial Sequence
  R2_5′RNA - 50bp 5′UTR and 117aa of R2 ORF
tccggaaacaggtgggccggggcgccaccaggggggagcaatccctcctgatgatggcgagcaccgcactgtcccttatgggacggtgtaacccggatggctgtacacgtggtaaacacgtgacagcagccccgatggacggaccgcgaggaccgtcaagcctagcaggtaccttcgggtggggccttgcgatacctgcgggcgaaccctgtggtcgggtttgcagcccggccacagtgggtttttttcctgttgcaaaaaagtcaaataaagaaaatagacctgaagcctctggcctcccgctggagtcagagaggacaggcgataacccgactgtgcggggttccgccggcgcagatcctgtgggtcaggatgcgcctggttggacctgccagttctgc

SEQ ID NO: 43 - DNA, 679 bp, Artificial Sequence
  R2OI_5′R2OI RNA - sequence required for protein binding composed of 5′UTR and first 138aa of the R2OI ORF
cgcacaggggacacagagcctgcccaagtaccgctcccgagggagcgggaaacgggggggtgactatcccctggggtccggcgagagcgctggtctacggaccaggggtggctgtgggcaggctgctcctcaggccagttgattagttacgcatgggctgtacctccacgtggtcccgctggtaacgacttgtcggctaaatcagcccgcccaccatctgggatatggttgaccgtctaaccccagtactcaggtcacaaacaaaatgggaacagatacagtgtatgtcggccaggactacccttctggcttatcaaaacgggtaccagcacggttagtggcgggaccgatgctgcgagagcgaagctgtcacgcccatgtgtttagggctggacacatgtggaactggcgaaccagccttccgagcgggcgctgggaccagcccgctttggagaagtctcgggtcctaacccggtcggtggcgacggccaccgaccccgaaattacctcttacccaggaaagtccgtatcgacaagtacgcaggttcaggaggaggactggtgtagccgggagagcgggtggatctcgccaggacttgctcctgaagaaccctcggtggtgtccgaaattacagcctccatggtagcgacaatgagggtagcaaccgaggaggtcgtg

SEQ ID NO: 44 - DNA, 1381 bp, Artificial Sequence
  Insert sequence - hPGK-eGFP-SV40 poly(A) signal
gggttgcgccttttccaaggcagccctgggtttgcgcagggacgcggctgctctgggcgtggttccgggaaacgcagcggcgccgaccctgggtctcgcacattcttcacgtccgttcgcagcgtcacccggatcttcgccgctacccttgtgggccccccggcgacgcttcctgctccgcccctaagtcgggaaggttccttgcggttcgcggcgtgccggacgtgacaaacggaagccgcacgtctcactagtaccctcgcagacggacagcgccagggagcaatggcagcgcgccgaccgcgatgggctgtggccaatagcggctgctcagcagggcgcgccgagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctccccaggcaagtttgtacaaaaaagcaggctgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaaaacaacttgtttattgcagcttataatggttacaaataaagcaatagcatcacaaatttcacaaataaagcatttttttcactgcattctagttgtggtttgtccaaactcatcaatgtatctta

SEQ ID NO: 45 - DNA, 2258 bp, Artificial Sequence
  Fusion of spScaffold, 106bp homology arm, R2_5′RNA, hPGK-GFP-SV40 polyA, R2_3′RNA, 30bp - 3′ homology arm
tttaagagctatgctggaaacagcatagcaagtttaaataaggctagtccgttatcaacttgaaaaagtggcaccgagtcggtgctttttttgcgggtgttgacgcgatgtgatttctgcccagtgctctgaatgtcaaagtgaagaaattcaatgaagcgcgggtaaacggcgggagtaactatgactctcttaaggtccggaaacaggtgggccggggcgccaccaggggggagcaatccctcctgatgatggcgagcaccgcactgtcccttatgggacggtgtaacccggatggctgtacacgtggtaaacacgtgacagcagccccgatggacggaccgcgaggaccgtcaagcctagcaggtaccttcgggtggggccttgcgatacctgcgggcgaaccctgtggtcgggtttgcagcccggccacagtgggtttttttcctgttgcaaaaaagtcaaataaagaaaatagacctgaagcctctggcctcccgctggagtcagagaggacaggcgataacccgactgtgcggggttccgccggcgcagatcctgtgggtcaggatgcgcctggttggacctgccagttctgcgggttgcgccttttccaaggcagccctgggtttgcgcagggacgcggctgctctgggcgtggttccgggaaacgcagcggcgccgaccctgggtctcgcacattcttcacgtccgttcgcagcgtcacccggatcttcgccgctacccttgtgggccccccggcgacgcttcctgctccgcccctaagtcgggaaggttccttgcggttcgcggcgtgccggacgtgacaaacggaagccgcacgtctcactagtaccctcgcagacggacagcgccagggagcaatggcagcgcgccgaccgcgatgggctgtggccaatagcggctgctcagcagggcgcgccgagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctccccaggcaagtttgtacaaaaaagcaggctgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaaaacaacttgtttattgcagcttataatggttacaaataaagcaatagcatcacaaatttcacaaataaagcatttttttcactgcattctagttgtggtttgtccaaactcatcaatgtatcttagccttgcacagtagtccagcggtaagggtgtagatcaggcccgtctgtttctcccccggagctcgctcccttggcttcccttatatattttaacatcagaaacagacattaaacatctactgatccaatttcgccggcgtacggccacgatcgggagggtgggaatctcgggggtcttccgatcctaatccatgatgattacgacctgagtcactaaagacgatggcatgatgatccggcgatgaaaatagccaaatgcctcgtcatctaattagtga

SEQ ID NO: 46 - DNA, 2396 bp, Artificial Sequence
  Fusion of spScaffold, 106bp homology arm, R2OI_5′RNA, hPGK-GFP-SV40 polyA, R2OI_3′RNA, 30bp - 3′ homology arm
tttaagagctatgctggaaacagcatagcaagtttaaataaggctagtccgttatcaacttgaaaaagtggcaccgagtcggtgctttttttgcgggtgttgacgcgatgtgatttctgcccagtgctctgaatgtcaaagtgaagaaattcaatgaagcgcgggtaaacggcgggagtaactatgactctcttaaggcgcacaggggacacagagcctgcccaagtaccgctcccgagggagcgggaaacgggggggtgactatcccctggggtccggcgagagcgctggtctacggaccaggggtggctgtgggcaggctgctcctcaggccagttgattagttacgcatgggctgtacctccacgtggtcccgctggtaacgacttgtcggctaaatcagcccgcccaccatctgggatatggttgaccgtctaaccccagtactcaggtcacaaacaaaatgggaacagatacagtgtatgtcggccaggactacccttctggcttatcaaaacgggtaccagcacggttagtggcgggaccgatgctgcgagagcgaagctgtcacgcccatgtgtttagggctggacacatgtggaactggcgaaccagccttccgagcgggcgctgggaccagcccgctttggagaagtctcgggtcctaacccggtcggtggcgacggccaccgaccccgaaattacctcttacccaggaaagtccgtatcgacaagtacgcaggttcaggaggaggactggtgtagccgggagagcgggtggatctcgccaggacttgctcctgaagaaccctcggtggtgtccgaaattacagcctccatggtagcgacaatgagggtagcaaccgaggaggtcgtggggttgcgccttttccaaggcagccctgggtttgcgcagggacgcggctgctctgggcgtggttccgggaaacgcagcggcgccgaccctgggtctcgcacattcttcacgtccgttcgcagcgtcacccggatcttcgccgctacccttgtgggccccccggcgacgcttcctgctccgcccctaagtcgggaaggttccttgcggttcgcggcgtgccggacgtgacaaacggaagccgcacgtctcactagtaccctcgcagacggacagcgccagggagcaatggcagcgcgccgaccgcgatgggctgtggccaatagcggctgctcagcagggcgcgccgagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctccccaggcaagtttgtacaaaaaagcaggctgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaaaacaacttgtttattgcagcttataatggttacaaataaagcaatagcatcacaaatttcacaaataaagcatttttttcactgcattctagttgtggtttgtccaaactcatcaatgtatcttagggggacagctgggagtctcggcatgattacaaatcttgcgctgcactcggatgtcgtccccgtgacggacacattaatccggaaagcgagtggtgactcgcctcaagtagccaaatgcctcgtcatctaattagtga

SEQ ID NO: 47 - DNA, 2279 bp, Artificial Sequence
  Fusion of SPACER, spScaffold, 106bp homology arm, R2_5′RNA, hPGK-GFP-SV40 polyA, R2_3′RNA, 30bp - 3′ homology arm
taattagtgacgcgcatgaagtttaagagctatgctggaaacagcatagcaagtttaaataaggctagtccgttatcaacttgaaaaagtggcaccgagtcggtgctttttttgcgggtgttgacgcgatgtgatttctgcccagtgctctgaatgtcaaagtgaagaaattcaatgaagcgcgggtaaacggcgggagtaactatgactctcttaaggtccggaaacaggtgggccggggcgccaccaggggggagcaatccctcctgatgatggcgagcaccgcactgtcccttatgggacggtgtaacccggatggctgtacacgtggtaaacacgtgacagcagccccgatggacggaccgcgaggaccgtcaagcctagcaggtaccttcgggtggggccttgcgatacctgcgggcgaaccctgtggtcgggtttgcagcccggccacagtgggtttttttcctgttgcaaaaaagtcaaataaagaaaatagacctgaagcctctggcctcccgctggagtcagagaggacaggcgataacccgactgtgcggggttccgccggcgcagatcctgtgggtcaggatgcgcctggttggacctgccagttctgcgggttgcgccttttccaaggcagccctgggtttgcgcagggacgcggctgctctgggcgtggttccgggaaacgcagcggcgccgaccctgggtctcgcacattcttcacgtccgttcgcagcgtcacccggatcttcgccgctacccttgtgggccccccggcgacgcttcctgctccgcccctaagtcgggaaggttccttgcggttcgcggcgtgccggacgtgacaaacggaagccgcacgtctcactagtaccctcgcagacggacagcgccagggagcaatggcagcgcgccgaccgcgatgggctgtggccaatagcggctgctcagcagggcgcgccgagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctccccaggcaagtttgtacaaaaaagcaggctgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaaaacaacttgtttattgcagcttataatggttacaaataaagcaatagcatcacaaatttcacaaataaagcatttttttcactgcattctagttgtggtttgtccaaactcatcaatgtatcttagccttgcacagtagtccagcggtaagggtgtagatcaggcccgtctgtttctcccccggagctcgctcccttggcttcccttatatattttaacatcagaaacagacattaaacatctactgatccaatttcgccggcgtacggccacgatcgggagggtgggaatctcgggggtcttccgatcctaatccatgatgattacgacctgagtcactaaagacgatggcatgatgatccggcgatgaaaatagccaaatgcctcgtcatctaattagtga

SEQ ID NO: 48 - DNA, 2417 bp, Artificial Sequence
  Fusion of SPACER, spScaffold, 106bp homology arm, R2OI_5′RNA, hPGK-GFP-SV40 polyA, R2OI_3′RNA, 30bp - 3′ homology arm
taattagtgacgcgcatgaagtttaagagctatgctggaaacagcatagcaagtttaaataaggctagtccgttatcaacttgaaaaagtggcaccgagtcggtgctttttttgcgggtgttgacgcgatgtgatttctgcccagtgctctgaatgtcaaagtgaagaaattcaatgaagcgcgggtaaacggcgggagtaactatgactctcttaaggcgcacaggggacacagagcctgcccaagtaccgctcccgagggagcgggaaacgggggggtgactatcccctggggtccggcgagagcgctggtctacggaccaggggtggctgtgggcaggctgctcctcaggccagttgattagttacgcatgggctgtacctccacgtggtcccgctggtaacgacttgtcggctaaatcagcccgcccaccatctgggatatggttgaccgtctaaccccagtactcaggtcacaaacaaaatgggaacagatacagtgtatgtcggccaggactacccttctggcttatcaaaacgggtaccagcacggttagtggcgggaccgatgctgcgagagcgaagctgtcacgcccatgtgtttagggctggacacatgtggaactggcgaaccagccttccgagcgggcgctgggaccagcccgctttggagaagtctcgggtcctaacccggtcggtggcgacggccaccgaccccgaaattacctcttacccaggaaagtccgtatcgacaagtacgcaggttcaggaggaggactggtgtagccgggagagcgggtggatctcgccaggacttgctcctgaagaaccctcggtggtgtccgaaattacagcctccatggtagcgacaatgagggtagcaaccgaggaggtcgtggggttgcgccttttccaaggcagccctgggtttgcgcagggacgcggctgctctgggcgtggttccgggaaacgcagcggcgccgaccctgggtctcgcacattcttcacgtccgttcgcagcgtcacccggatcttcgccgctacccttgtgggccccccggcgacgcttcctgctccgcccctaagtcgggaaggttccttgcggttcgcggcgtgccggacgtgacaaacggaagccgcacgtctcactagtaccctcgcagacggacagcgccagggagcaatggcagcgcgccgaccgcgatgggctgtggccaatagcggctgctcagcagggcgcgccgagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctccccaggcaagtttgtacaaaaaagcaggctgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaaaacaacttgtttattgcagcttataatggttacaaataaagcaatagcatcacaaatttcacaaataaagcatttttttcactgcattctagttgtggtttgtccaaactcatcaatgtatcttagggggacagctgggagtctcggcatgattacaaatcttgcgctgcactcggatgtcgtccccgtgacggacacattaatccggaaagcgagtggtgactcgcctcaagtagccaaatgcctcgtcatctaattagtga

SEQ ID NO: 49 - DNA, 2278 bp, Artificial Sequence
  Fusion of OMNI50_ TracrRNA, 106bp homology arm, R2_5′RNA, hPGK-GFP-SV40 polyA, R2_3′RNA, 30bp - 3′ homology arm
gtttgagagttatgtaagaaattacatgacgagttcaaataaaaatttattcaaaccgcctatttataggccgcagatgttctgcattatgcttgctattgcaagcttttttgcgggtgttgacgcgatgtgatttctgcccagtgctctgaatgtcaaagtgaagaaattcaatgaagcgcgggtaaacggcgggagtaactatgactctcttaaggtccggaaacaggtgggccggggcgccaccaggggggagcaatccctcctgatgatggcgagcaccgcactgtcccttatgggacggtgtaacccggatggctgtacacgtggtaaacacgtgacagcagccccgatggacggaccgcgaggaccgtcaagcctagcaggtaccttcgggtggggccttgcgatacctgcgggcgaaccctgtggtcgggtttgcagcccggccacagtgggtttttttcctgttgcaaaaaagtcaaataaagaaaatagacctgaagcctctggcctcccgctggagtcagagaggacaggcgataacccgactgtgcggggttccgccggcgcagatcctgtgggtcaggatgcgcctggttggacctgccagttctgcgggttgcgccttttccaaggcagccctgggtttgcgcagggacgcggctgctctgggcgtggttccgggaaacgcagcggcgccgaccctgggtctcgcacattcttcacgtccgttcgcagcgtcacccggatcttcgccgctacccttgtgggccccccggcgacgcttcctgctccgcccctaagtcgggaaggttccttgcggttcgcggcgtgccggacgtgacaaacggaagccgcacgtctcactagtaccctcgcagacggacagcgccagggagcaatggcagcgcgccgaccgcgatgggctgtggccaatagcggctgctcagcagggcgcgccgagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctccccaggcaagtttgtacaaaaaagcaggctgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaaaacaacttgtttattgcagcttataatggttacaaataaagcaatagcatcacaaatttcacaaataaagcatttttttcactgcattctagttgtggtttgtccaaactcatcaatgtatcttagccttgcacagtagtccagcggtaagggtgtagatcaggcccgtctgtttctcccccggagctcgctcccttggcttcccttatatattttaacatcagaaacagacattaaacatctactgatccaatttcgccggcgtacggccacgatcgggagggtgggaatctcgggggtcttccgatcctaatccatgatgattacgacctgagtcactaaagacgatggcatgatgatccggcgatgaaaatagccaaatgcctcgtcatctaattagtga

SEQ ID NO: 50 - DNA, 2416 bp, Artificial Sequence
  Fusion of OMNI50_Scaffold, 106bp homology arm, R2OI_5′RNA, hPGK-GFP-SV40 polyA, R2OI_3′RNA, 30bp - 3′ homology arm
gtttgagagttatgtaagaaattacatgacgagttcaaataaaaatttattcaaaccgcctatttataggccgcagatgttctgcattatgcttgctattgcaagcttttttgcgggtgttgacgcgatgtgatttctgcccagtgctctgaatgtcaaagtgaagaaattcaatgaagcgcgggtaaacggcgggagtaactatgactctcttaaggcgcacaggggacacagagcctgcccaagtaccgctcccgagggagcgggaaacgggggggtgactatcccctggggtccggcgagagcgctggtctacggaccaggggtggctgtgggcaggctgctcctcaggccagttgattagttacgcatgggctgtacctccacgtggtcccgctggtaacgacttgtcggctaaatcagcccgcccaccatctgggatatggttgaccgtctaaccccagtactcaggtcacaaacaaaatgggaacagatacagtgtatgtcggccaggactacccttctggcttatcaaaacgggtaccagcacggttagtggcgggaccgatgctgcgagagcgaagctgtcacgcccatgtgtttagggctggacacatgtggaactggcgaaccagccttccgagcgggcgctgggaccagcccgctttggagaagtctcgggtcctaacccggtcggtggcgacggccaccgaccccgaaattacctcttacccaggaaagtccgtatcgacaagtacgcaggttcaggaggaggactggtgtagccgggagagcgggtggatctcgccaggacttgctcctgaagaaccctcggtggtgtccgaaattacagcctccatggtagcgacaatgagggtagcaaccgaggaggtcgtggggttgcgccttttccaaggcagccctgggtttgcgcagggacgcggctgctctgggcgtggttccgggaaacgcagcggcgccgaccctgggtctcgcacattcttcacgtccgttcgcagcgtcacccggatcttcgccgctacccttgtgggccccccggcgacgcttcctgctccgcccctaagtcgggaaggttccttgcggttcgcggcgtgccggacgtgacaaacggaagccgcacgtctcactagtaccctcgcagacggacagcgccagggagcaatggcagcgcgccgaccgcgatgggctgtggccaatagcggctgctcagcagggcgcgccgagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctccccaggcaagtttgtacaaaaaagcaggctgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaaaacaacttgtttattgcagcttataatggttacaaataaagcaatagcatcacaaatttcacaaataaagcatttttttcactgcattctagttgtggtttgtccaaactcatcaatgtatcttagggggacagctgggagtctcggcatgattacaaatcttgcgctgcactcggatgtcgtccccgtgacggacacattaatccggaaagcgagtggtgactcgcctcaagtagccaaatgcctcgtcatctaattagtga

SEQ ID NO: 51 - DNA, 2300 bp, Artificial Sequence
  Fusion of SPACER, OMNI50_Scaffold, 106bp homology arm, R2_5′RNA, hPGK-GFP-SV40 polyA, R2_3′RNA, 30bp - 3′ homology arm
agaagcctgatgttagaatcaagtttgagagttatgtaagaaattacatgacgagttcaaataaaaatttattcaaaccgcctatttataggccgcagatgttctgcattatgcttgctattgcaagcttttttgcgggtgttgacgcgatgtgatttctgcccagtgctctgaatgtcaaagtgaagaaattcaatgaagcgcgggtaaacggcgggagtaactatgactctcttaaggtccggaaacaggtgggccggggcgccaccaggggggagcaatccctcctgatgatggcgagcaccgcactgtcccttatgggacggtgtaacccggatggctgtacacgtggtaaacacgtgacagcagccccgatggacggaccgcgaggaccgtcaagcctagcaggtaccttcgggtggggccttgcgatacctgcgggcgaaccctgtggtcgggtttgcagcccggccacagtgggtttttttcctgttgcaaaaaagtcaaataaagaaaatagacctgaagcctctggcctcccgctggagtcagagaggacaggcgataacccgactgtgcggggttccgccggcgcagatcctgtgggtcaggatgcgcctggttggacctgccagttctgcgggttgcgccttttccaaggcagccctgggtttgcgcagggacgcggctgctctgggcgtggttccgggaaacgcagcggcgccgaccctgggtctcgcacattcttcacgtccgttcgcagcgtcacccggatcttcgccgctacccttgtgggccccccggcgacgcttcctgctccgcccctaagtcgggaaggttccttgcggttcgcggcgtgccggacgtgacaaacggaagccgcacgtctcactagtaccctcgcagacggacagcgccagggagcaatggcagcgcgccgaccgcgatgggctgtggccaatagcggctgctcagcagggcgcgccgagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctccccaggcaagtttgtacaaaaaagcaggctgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaaaacaacttgtttattgcagcttataatggttacaaataaagcaatagcatcacaaatttcacaaataaagcatttttttcactgcattctagttgtggtttgtccaaactcatcaatgtatcttagccttgcacagtagtccagcggtaagggtgtagatcaggcccgtctgtttctcccccggagctcgctcccttggcttcccttatatattttaacatcagaaacagacattaaacatctactgatccaatttcgccggcgtacggccacgatcgggagggtgggaatctcgggggtcttccgatcctaatccatgatgattacgacctgagtcactaaagacgatggcatgatgatccggcgatgaaaatagccaaatgcctcgtcatctaattagtga

SEQ ID NO: 52 - DNA, 2438 bp, Artificial Sequence
  Fusion of SPACER OMNI50_Scaffold, 106bp homology arm, R2OI_5′RNA, hPGK-GFP-SV40 polyA, R2OI_3′RNA, 30bp - 3′ homology arm
agaagcctgatgttagaatcaagtttgagagttatgtaagaaattacatgacgagttcaaataaaaatttattcaaaccgcctatttataggccgcagatgttctgcattatgcttgctattgcaagcttttttgcgggtgttgacgcgatgtgatttctgcccagtgctctgaatgtcaaagtgaagaaattcaatgaagcgcgggtaaacggcgggagtaactatgactctcttaaggcgcacaggggacacagagcctgcccaagtaccgctcccgagggagcgggaaacgggggggtgactatcccctggggtccggcgagagcgctggtctacggaccaggggtggctgtgggcaggctgctcctcaggccagttgattagttacgcatgggctgtacctccacgtggtcccgctggtaacgacttgtcggctaaatcagcccgcccaccatctgggatatggttgaccgtctaaccccagtactcaggtcacaaacaaaatgggaacagatacagtgtatgtcggccaggactacccttctggcttatcaaaacgggtaccagcacggttagtggcgggaccgatgctgcgagagcgaagctgtcacgcccatgtgtttagggctggacacatgtggaactggcgaaccagccttccgagcgggcgctgggaccagcccgctttggagaagtctcgggtcctaacccggtcggtggcgacggccaccgaccccgaaattacctcttacccaggaaagtccgtatcgacaagtacgcaggttcaggaggaggactggtgtagccgggagagcgggtggatctcgccaggacttgctcctgaagaaccctcggtggtgtccgaaattacagcctccatggtagcgacaatgagggtagcaaccgaggaggtcgtggggttgcgccttttccaaggcagccctgggtttgcgcagggacgcggctgctctgggcgtggttccgggaaacgcagcggcgccgaccctgggtctcgcacattcttcacgtccgttcgcagcgtcacccggatcttcgccgctacccttgtgggccccccggcgacgcttcctgctccgcccctaagtcgggaaggttccttgcggttcgcggcgtgccggacgtgacaaacggaagccgcacgtctcactagtaccctcgcagacggacagcgccagggagcaatggcagcgcgccgaccgcgatgggctgtggccaatagcggctgctcagcagggcgcgccgagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctccccaggcaagtttgtacaaaaaagcaggctgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaaaacaacttgtttattgcagcttataatggttacaaataaagcaatagcatcacaaatttcacaaataaagcatttttttcactgcattctagttgtggtttgtccaaactcatcaatgtatcttagggggacagctgggagtctcggcatgattacaaatcttgcgctgcactcggatgtcgtccccgtgacggacacattaatccggaaagcgagtggtgactcgcctcaagtagccaaatgcctcgtcatctaattagtga

SEQ ID NO: 53 - DNA, 4107 bp, Artificial Sequence
  nCas9 (D10A) codon optimized for human
atggacaagaagtatagcatcggcctggccatcggcacaaactccgtgggctgggccgtgatcaccgacgagtacaaggtgccaagcaagaagtttaaggtgctgggcaacaccgatagacactccatcaagaagaatctgatcggcgccctgctgttcgactctggcgagacagccgaggccacacggctgaagagaaccgcccggagaaggtatacacgccggaagaataggatctgctacctgcaggagatcttcagcaacgagatggccaaggtggacgattctttctttcaccgcctggaggagagcttcctggtggaggaggataagaagcacgagcggcaccctatctttggcaacatcgtggacgaggtggcctatcacgagaagtacccaacaatctatcacctgaggaagaagctggtggactccaccgataaggccgacctgcgcctgatctatctggccctggcccacatgatcaagttccggggccactttctgatcgagggcgatctgaacccagacaatagcgatgtggacaagctgttcatccagctggtgcagacctacaatcagctgtttgaggagaaccccatcaatgcctctggagtggacgcaaaggcaatcctgagcgccagactgtccaagtctagaaggctggagaacctgatcgcccagctgccaggcgagaagaagaacggcctgtttggcaatctgatcgccctgtccctgggcctgacacccaacttcaagtctaattttgatctggccgaggacgccaagctgcagctgtccaaggacacctatgacgatgacctggataacctgctggcccagatcggcgatcagtacgccgacctgttcctggccgccaagaatctgtctgacgccatcctgctgagcgatatcctgcgcgtgaacaccgagatcacaaaggcccccctgagcgcctccatgatcaagagatatgacgagcaccaccaggatctgaccctgctgaaggccctggtgaggcagcagctgcctgagaagtacaaggagatcttctttgatcagagcaagaatggatacgcaggatatatcgacggaggagcatcccaggaggagttctacaagtttatcaagcctatcctggagaagatggacggcacagaggagctgctggtgaagctgaatcgggaggacctgctgaggaagcagcgcacctttgataacggcagcatccctcaccagatccacctgggagagctgcacgcaatcctgcgccggcaggaggacttctacccatttctgaaggataaccgggagaagatcgagaagatcctgacattcagaatcccctactatgtgggacctctggcccggggcaatagcagatttgcctggatgacccgcaagtccgaggagacaatcacaccctggaacttcgaggaggtggtggataagggcgcctctgcccagagcttcatcgagcggatgaccaattttgacaagaacctgcctaatgagaaggtgctgccaaagcactctctgctgtacgagtatttcaccgtgtataacgagctgacaaaggtgaagtacgtgaccgagggcatgagaaagcctgccttcctgagcggcgagcagaagaaggccatcgtggacctgctgtttaagaccaataggaaggtgacagtgaagcagctgaaggaggactatttcaagaagatcgagtgttttgattctgtggagatcagcggcgtggaggacaggtttaacgcctccctgggcacctaccacgatctgctgaagatcatcaaggataaggacttcctggacaacgaggagaatgaggatatcctggaggacatcgtgctgaccctgacactgtttgaggatagggagatgatcgaggagcgcctgaagacatatgcccacctgttcgatgacaaagtgatgaagcagctgaagagaaggcgctacaccggatggggccggctgagcagaaagctgatcaatggcatccgcgacaagcagtctggcaagacaatcctggactttctgaagagcgatggcttcgccaaccggaacttcatgcagctgatccacgatgactccctgaccttcaaggaggatatccagaaggcacaggtgtctggacagggcgacagcctgcacgagcacatcgccaacctggccggctctcctgccatcaagaagggcatcctgcagaccgtgaaggtggtggacgagctggtgaaagtgatgggcaggcacaagccagagaacatcgtgatcgagatggcccgcgagaatcagaccacacagaagggccagaagaactcccgggagagaatgaagagaatcgaggagggcatcaaggagctgggctctcagatcctgaaggagcaccccgtggagaacacacagctgcagaatgagaagctgtatctgtactatctgcagaatggccgggatatgtacgtggaccaggagctggatatcaacagactgtctgattatgacgtggatcacatcgtgccacagtccttcctgaaggatgactctatcgacaataaggtgctgaccaggagcgacaagaaccgcggcaagtccgataatgtgccctctgaggaggtggtgaagaagatgaagaactactggaggcagctgctgaatgccaagctgatcacacagaggaagtttgataacctgaccaaggcagagaggggaggactgtccgagctggacaaggccggcttcatcaagcggcagctggtggagacaagacagatcacaaagcacgtggcccagatcctggattctagaatgaacacaaagtacgatgagaatgacaagctgatcagggaggtgaaagtgatcaccctgaagtccaagctggtgtctgactttaggaaggatttccagttttataaggtgcgcgagatcaacaattatcaccacgcccacgacgcctacctgaacgccgtggtgggcacagccctgatcaagaagtaccctaagctggagtccgagttcgtgtacggcgactataaggtgtacgatgtgcgcaagatgatcgccaagtctgagcaggagatcggcaaggccaccgccaagtatttcttttacagcaacatcatgaatttctttaagaccgagatcacactggccaatggcgagatcaggaagcgcccactgatcgagacaaacggcgagacaggcgagatcgtgtgggacaagggcagggattttgccaccgtgcgcaaggtgctgagcatgccccaagtgaatatcgtgaagaagaccgaggtgcagacaggcggcttctccaaggagtctatcctgcctaagcggaactccgataagctgatcgccagaaagaaggactgggaccccaagaagtatggcggcttcgacagccctacagtggcctactccgtgctggtggtggccaaggtggagaagggcaagagcaagaagctgaagtccgtgaaggagctgctgggcatcaccatcatggagcgcagctccttcgagaagaatcctatcgactttctggaggccaagggctataaggaggtgaagaaggacctgatcatcaagctgccaaagtactctctgtttgagctggagaacggaaggaagagaatgctggcaagcgccggagagctgcagaagggcaatgagctggccctgccctccaagtacgtgaacttcctgtatctggcctcccactacgagaagctgaagggctctcctgaggataacgagcagaagcagctgtttgtggagcagcacaagcactatctggacgagatcatcgagcagatcagcgagttctccaagagagtgatcctggccgacgccaatctggataaggtgctgtccgcctacaacaagcaccgggataagccaatcagagagcaggccgagaatatcatccacctgtttaccctgacaaacctgggagcaccagcagccttcaagtattttgacaccacaatcgacaggaagcggtacaccagcacaaaggaggtgctggacgccacactgatccaccagtccatcaccggcctgtacgagacacggatcgacctgtctcagctgggaggcgattga

SEQ ID NO: 54 - DNA, 4107 bp, Artificial Sequence
  nCas9 (N863A) codon optimized for human
atggacaagaagtatagcatcggcctggatatcggcacaaactccgtgggctgggccgtgatcaccgacgagtacaaggtgccaagcaagaagtttaaggtgctgggcaacaccgatagacactccatcaagaagaatctgatcggcgccctgctgttcgactctggcgagacagccgaggccacacggctgaagagaaccgcccggagaaggtatacacgccggaagaataggatctgctacctgcaggagatcttcagcaacgagatggccaaggtggacgattctttctttcaccgcctggaggagagcttcctggtggaggaggataagaagcacgagcggcaccctatctttggcaacatcgtggacgaggtggcctatcacgagaagtacccaacaatctatcacctgaggaagaagctggtggactccaccgataaggccgacctgcgcctgatctatctggccctggcccacatgatcaagttccggggccactttctgatcgagggcgatctgaacccagacaatagcgatgtggacaagctgttcatccagctggtgcagacctacaatcagctgtttgaggagaaccccatcaatgcctctggagtggacgcaaaggcaatcctgagcgccagactgtccaagtctagaaggctggagaacctgatcgcccagctgccaggcgagaagaagaacggcctgtttggcaatctgatcgccctgtccctgggcctgacacccaacttcaagtctaattttgatctggccgaggacgccaagctgcagctgtccaaggacacctatgacgatgacctggataacctgctggcccagatcggcgatcagtacgccgacctgttcctggccgccaagaatctgtctgacgccatcctgctgagcgatatcctgcgcgtgaacaccgagatcacaaaggcccccctgagcgcctccatgatcaagagatatgacgagcaccaccaggatctgaccctgctgaaggccctggtgaggcagcagctgcctgagaagtacaaggagatcttctttgatcagagcaagaatggatacgcaggatatatcgacggaggagcatcccaggaggagttctacaagtttatcaagcctatcctggagaagatggacggcacagaggagctgctggtgaagctgaatcgggaggacctgctgaggaagcagcgcacctttgataacggcagcatccctcaccagatccacctgggagagctgcacgcaatcctgcgccggcaggaggacttctacccatttctgaaggataaccgggagaagatcgagaagatcctgacattcagaatcccctactatgtgggacctctggcccggggcaatagcagatttgcctggatgacccgcaagtccgaggagacaatcacaccctggaacttcgaggaggtggtggataagggcgcctctgcccagagcttcatcgagcggatgaccaattttgacaagaacctgcctaatgagaaggtgctgccaaagcactctctgctgtacgagtatttcaccgtgtataacgagctgacaaaggtgaagtacgtgaccgagggcatgagaaagcctgccttcctgagcggcgagcagaagaaggccatcgtggacctgctgtttaagaccaataggaaggtgacagtgaagcagctgaaggaggactatttcaagaagatcgagtgttttgattctgtggagatcagcggcgtggaggacaggtttaacgcctccctgggcacctaccacgatctgctgaagatcatcaaggataaggacttcctggacaacgaggagaatgaggatatcctggaggacatcgtgctgaccctgacactgtttgaggatagggagatgatcgaggagcgcctgaagacatatgcccacctgttcgatgacaaagtgatgaagcagctgaagagaaggcgctacaccggatggggccggctgagcagaaagctgatcaatggcatccgcgacaagcagtctggcaagacaatcctggactttctgaagagcgatggcttcgccaaccggaacttcatgcagctgatccacgatgactccctgaccttcaaggaggatatccagaaggcacaggtgtctggacagggcgacagcctgcacgagcacatcgccaacctggccggctctcctgccatcaagaagggcatcctgcagaccgtgaaggtggtggacgagctggtgaaagtgatgggcaggcacaagccagagaacatcgtgatcgagatggcccgcgagaatcagaccacacagaagggccagaagaactcccgggagagaatgaagagaatcgaggagggcatcaaggagctgggctctcagatcctgaaggagcaccccgtggagaacacacagctgcagaatgagaagctgtatctgtactatctgcagaatggccgggatatgtacgtggaccaggagctggatatcaacagactgtctgattatgacgtggatcacatcgtgccacagtccttcctgaaggatgactctatcgacaataaggtgctgaccaggagcgacaaggcccgcggcaagtccgataatgtgccctctgaggaggtggtgaagaagatgaagaactactggaggcagctgctgaatgccaagctgatcacacagaggaagtttgataacctgaccaaggcagagaggggaggactgtccgagctggacaaggccggcttcatcaagcggcagctggtggagacaagacagatcacaaagcacgtggcccagatcctggattctagaatgaacacaaagtacgatgagaatgacaagctgatcagggaggtgaaagtgatcaccctgaagtccaagctggtgtctgactttaggaaggatttccagttttataaggtgcgcgagatcaacaattatcaccacgcccacgacgcctacctgaacgccgtggtgggcacagccctgatcaagaagtaccctaagctggagtccgagttcgtgtacggcgactataaggtgtacgatgtgcgcaagatgatcgccaagtctgagcaggagatcggcaaggccaccgccaagtatttcttttacagcaacatcatgaatttctttaagaccgagatcacactggccaatggcgagatcaggaagcgcccactgatcgagacaaacggcgagacaggcgagatcgtgtgggacaagggcagggattttgccaccgtgcgcaaggtgctgagcatgccccaagtgaatatcgtgaagaagaccgaggtgcagacaggcggcttctccaaggagtctatcctgcctaagcggaactccgataagctgatcgccagaaagaaggactgggaccccaagaagtatggcggcttcgacagccctacagtggcctactccgtgctggtggtggccaaggtggagaagggcaagagcaagaagctgaagtccgtgaaggagctgctgggcatcaccatcatggagcgcagctccttcgagaagaatcctatcgactttctggaggccaagggctataaggaggtgaagaaggacctgatcatcaagctgccaaagtactctctgtttgagctggagaacggaaggaagagaatgctggcaagcgccggagagctgcagaagggcaatgagctggccctgccctccaagtacgtgaacttcctgtatctggcctcccactacgagaagctgaagggctctcctgaggataacgagcagaagcagctgtttgtggagcagcacaagcactatctggacgagatcatcgagcagatcagcgagttctccaagagagtgatcctggccgacgccaatctggataaggtgctgtccgcctacaacaagcaccgggataagccaatcagagagcaggccgagaatatcatccacctgtttaccctgacaaacctgggagcaccagcagccttcaagtattttgacaccacaatcgacaggaagcggtacaccagcacaaaggaggtgctggacgccacactgatccaccagtccatcaccggcctgtacgagacacggatcgacctgtctcagctgggaggcgattga

SEQ ID NO: 55 - DNA, 4200 bp, Artificial Sequence
  OMNI50 - nuclease sequence with HA-tag and NLS
atgcctaagaagaagagaaaggtgggtaccaccaaggtgaaggactactacataggcttggacatcggcacctctagcgtcgggtgggccgtcaccgatgaagcctataacgtgcttaagtttaatagcaagaaaatgtggggcgtgcggctgttcgacgacgctaagacggcagaggagcgtaggggccagcgaggagcaagacgacgtctggatcggaagaaggagagactcagcctgctgcaggacttcttcgccgaagaggtagcaaaggtcgaccccaacttcttcctcaggctggacaattccgatctgtacatggaagataaggaccagaaactgaaaagcaaatatacactgttcaacgacaaggacttcaaggataagaattttcataagaagtaccccacaatacatcacctgctgatggatctgatcgaggacgacagtaagaaggacatccggctcgtctacctggcctgtcactatttgctcaagaacaggggtcatttcatcttcgagggccagaagttcgacactaaatcaagcttcgagaacagtttgaacgagctcaaagttcatttgaacgacgagtatggactggacctcgaatttgacaacgagaacctgattaacatcttgactgacccaaaactcaataaaacggccaagaagaaggagctgaagtccgtaatcggcgacaccaagttcctcaaagccgtttccgcgataatgatcggctctagccagaaactcgtcgacttgttcgagaaccccgaggatttcgacgactctgcgataaagtccgttgacttctcaactacctctttcgacgacaagtactctgactatgaactcgctctgggtgacaagatcgctctggtcaacatccttaaggaaatttacgatagctccatcctcgagaacctgctcaaagaggcagacaagtctaaggacggtaacaaatatatcagtaatgcattcgtgaagaagtacaataaacacggacaagatctgaaagagttcaaacgtctggtacgacaatatcacaagagtgcgtattttgatattttcagatccgagaaggtgaatgacaattacgtcagctacactaaaagctcaattagcaacaataaacgcgtcaaagcaaacaagttcactgatcaagaggccttctacaaattcgccaagaaacatctggagacaatcaagtataagatcaacaaggtaaacggctccaaggcagatctggagctgattgacgggatgctgcgggacatggagttcaagaactttatgcccaaaattaagtccagtgacaacggggtgattccataccagctcaagctgatggaattgaacaaaatactcgagaatcagtcaaagcatcacgagttcctcaatgtcagcgacgagtacggctccgtgtgtgataaaatcgcatctatcatggagttccgtatcccctactacgtgggacccctgaaccccaatagcaagtacgcctggatcaagaagcagaaagatagtgagattactccctggaacttcaaggacgtcgtggacctcgactccagcagagaggagttcattgactcactgatcggacgctgtacttaccttaaggacgagaaggtccttcccaaagcttctttgctgtataacgaatacatggtgctgaacgagctgaataacctgaagttgaacgaccttcccatcaccgaggagatgaagaagaagatatttgaccagttgttcaaaacaagaaagaaggtcacccttaaagcggtggcaaacctgctgaagaaggagttcaacatcaacggcgagattctgctctctgggaccgacggtgacttcaagcagggcttgaactcatacaatgacttcaaagctatcgtgggcgataaagtcgattccgatgattaccgggacaagattgaggagatcattaaactgatagttctttacggtgacgataagagttaccttcagaagaagattaaagctgggtatggaaaatacttcaccgacagtgagattaagaaaatggcggggctgaactacaaggattggggaaggctctcaaagaagctgctgacgggactcgagggtgcaaacaagatcactggagagcggggctccattattcacttcatgagggaatataaccttaatctgatggagcttatgtcagcttcatttacgttcaccgaagagatacagaaacttaaccccgtggatgaccgcaagctgtcatacgaaatggtggacgaactgtacctttctcccagtgtgaaacggatgctctggcagtccctgcgcatcgtcgacgagataaagaacatcatgggaaccgacagtaagaagattttcatcgagatggctcggggtaaggaagaggtgaaagcccgcaaggagtcaaggaagaaccaactgctgaagttctataaagacggaaagaaggcattcatcagcgagattggcgaggagaggtactcttacttgctttctgagatagagggtgaggaagagaataagtttcgatgggataacctgtacctttattatactcaactgggtcgctgcatgtactctttggaacctatcgacatatctgagctgtcttcaaagaatatttacgatcaggatcatatctaccccaaaagcaagatttacgacgacagtatcgagaatagggtgctggtgaagaaggaccttaactccaagaagggtaacagctatcctatcccagacgaaatcctgaacaagaactgttacgcctactggaagatcctgtacgataaaggtcttatcgggcagaagaagtacactcggctgacccggagaactggcttcacggacgacgagctcgttcagttcatctcaagacagatcgtggaaactagacaagcaacaaaggagactgctaacctgctcaagacaatatgtaagaactccgagatcgtgtattccaaagccgagaacgcaagtcggtttaggcaagagttcgacatcgtgaagtgtagggcggtgaacgatcttcatcatatgcacgatgcctacatcaacatcatagtggggaacgtgtataacaccaagttcacgaaggaccctatgaatttcgtaaagaagcaggaaaaggcgcggagctacaatctcgagaatatgttcaagtacgatgtgaaacgtggcggatacaccgcttggatcgccgatgacgagaagggcaccgtgaagaacgcgagtattaaacgtatccggaaggagctggaaggcacaaattataggttcacaagaatgaactacattgagtctggagcgcttttcaacgccactctccagcggaagaataagggctccagacccctgaaggacaaaggcccgaaatcttccatcgagaagtacggcggctacacaaacatcaataaagcctgtttcgctgttcttgacatcaagtctaagaacaagattgagaggaagctgatgcccgtcgagcgtgagatctatgccaaacagaagaacgacaagaagctgtccgacgagattttctcaaagtacctcaaggaccgatttggcatcgaggactacagggttgtctacccagtggtgaaaatgcgcacactgctcaagatcgacggcagctactacttcatcacaggcggttctgataagaccctggagttgcgatctgctctgcagctgattctccctaagaagaacgagtgggcgatcaaacagatcgacaagtcttccgaaaacgactatctgacgatcgagcgtatccaggacctgaccgaggagctggtgtataacactttcgacatcatcgtcaacaagttcaagaccagtgtcttcaagaagtctttccttaacttgtttcaggacgacaagattgagaacattgacttcaagtttaagtccatggacttcaaggagaaatgcaagacacttctcatgctggtcaaggcgattcgggcatccggcgtgaggcaggatctcaagtccatcgacctcaagtctgattacggacggctcagttcaaagaccaacaacatcggcaattaccaggagttcaagattattaatcagtccatcactggactgttcgagaatgaggtcgatctcctgaagctgggatcctacccatacgatgttccagattacgcggccgctccaaaaaagaaaagaaaagtttaa

SEQ ID NO: 56 - DNA, 3906 bp, Artificial Sequence
  R2OI (C248S, C251S, W294A) with HA-tag and NLS - R2OI mutant, point mutations in catalytic residues of DNA-binding domain
atgggcaccgacacagtgtacgtcggccaggattatcctagcggcctgagcaaaagagtgcccgctagactggttgctggccccatgctgagagagagatcttgtcacgcccacgtgttcagagccggacacatgtggaattggagaaccagcctgcctagcggcagatgggatcagcctgctctggaaaagtcccgggtgctgaccagatctgtggccaccgctacagaccccgagatcacatcttaccctggcaagagcgtgtccaccagcacacaggtgcaagaagaggactggtgtagcagagagagcggctggatttctcctggactggcccctgaggaacctagcgtggtgtctgagatcacagcctccatggtggccactatgagagtggctacagaggaagtggtgctggaacctcagcctgagcaggtcgtgacaattctgcccgagcacggcagaaatgtgccaccaggactggccgagcaggataccgcctctcctattgaagtgtccgtgctgctgcccgacctggccgaaaattgtcctctgtgtggtgttcccagcggcggactgagactgctgggaaagcactttgccgttagacatgccggcgtgcccgtgacctacgagtgtagaaagtgtgcctggcggagccccaatagccacagcatctcttgccacgtgccaaagtgcagaggcagagccagaatgccaagcggagatcctggaatcgccagcgatctgagcgaggccagatttgccacagaagtgggagtcgcccagcacaagagacacgtgcaccccgtggaatggaacaaagtgcggctggaaagaagaggcgccagaggcggaggaatcaaggccacaaaacttgccagcgtggccgaggtggaaaccctgatcagactgattagagagcacggcgatagcggcgccacataccagctgattgccgatgaactcggcagaggcaagacagccgagcaagtgcggagcaagaagcggctgctgagaatcgataccgccagcaactctcccgacgacgccgaagtggaagaggaaagactggaatctctggccgtgcggtccagcagcagatctcctcctagtctggtggctaccagagtgcgggaagctgtggcaaggggagaatctgaaggcggcgaggaaatcagagccattgccgcactgatcagagatgtggatcagaacccctgcctgatcgagacaagcgccagcgacatcatcagcaagctgggcagaagagtggacggccctaaaagacccagacctgtcgtgcgggaacagacccaagaaaaaggctgggtccgacggctggccagacggaagagagagtatagagaggcccagtacctgtacagcagagatcaggcaagactggccgctcagattctggatggcgctgcctctcaagaatgcgccctgcctgtggatcaagtgtacggcgccttccgggaaaagtgggagacagtgggacagtttcacggcctgggcgagtttagaacaggcgctagagccgacaactgggagttctactctcccatcctggctgccgaagtcaaagaaaacctgatgcggatggccaacggcacagcccctggacctgatagaatcagcaagaaggccctgctggactgggaccctagaggcgaacagctggctagactgtacaccacatggctgatcggcggcgtgatccccagagtgttcaaagagtgtcggaccaagctgctgcctaagagcagcgatcctgtggaactgcaggatatcggaggatggcggcctgtgacaatcggcagcatggtcaccagactgttcagcagaatcctgaccatgcggctgacccgggcctgtcctatcaatcctagacagagaggcttcctggccagcagctctggatgtgccgagaacctgctgatcttcgacgagatcgtgcggcggtctagaagagatggtggaccactggccgtggtgttcgtggatttcgccagagccttcgacagcatcagccacgagcacatcctgtgtgttctggaagaaggcggcctggatagacacgtgatcggcctgattcggaacagctacgtggactgtgtgaccagagtgggctgcgtggaaggcatgacacctccaatccagatgaaggtcggagtgaagcagggcgaccctatgagccctctgctgttcaatctggctatggaccctctgattcacaagctggaaacagccggcacaggcctgaagtggggagatctgtctatcgccacactggccttcgccgatgatctggtgctggtgtcagacagcgaagaaggcatgggcagatccctgggcatcctggaaaaattctgccagctgaccggcctgagagtgcagcctagaaagtgccacggcttcttcatggacaagggcgtcgtgaatggctgcggcacatgggagatttgtggcagccctatccacatgatcccaccaggcgaatctgtgcgctatctgggcgttcaagttggccctggaagaggcgtgatggaacccgatctgatccctaccgtgcacacctggatcgagagaatctctgaggcccctctgaagcccagccagagaatgagagtgctgaatagcttcgccctgccacggatcatctatcaggctgacctgggcaaagtgaccgtgacaaagctggcccagatcgatggaattgtgcggaaagccgtgaagaagtggctgcatctgagccccagcacctgtaatggcctgctgtactccagaaacagagatggcggactggggctcctgaagctggaacgactgattcctagcgtgcggaccaagagaatctaccggatgagcagaagccccgacatctggaccagaagaatgaccagccactccgtgtccaagagcgactgggaaatgctgtgggtgcaagctggcggagaaagaggctctgctcctgttatgggagccgtggaagccgctcctaccgatgtggaaagatcccctgactaccccgattggcggagagaggaaaatcttgcttggagcgccctgagagttcaaggcgtgggagctgatcagttcagaggcgatagaacctccagcagctggatcgccgaacctgcctctgtgggatttgcccagagacattggctggctgctctggcacttagagccggcgtgtaccctaccagagagtttctggccaggggcaaagaaaagagcggagccgcctgtagaagatgccctgccagactggaaagctgcagccacatcctgggccagtgtcctttcgtgcaggccaacagaatcgcccggcacaacaaagtgtgcgtgctcctggcaaccgaggccgagagatttggctggaccgtgatccgggaattccggcttgaagatgctgctggcgggctgaagattcccgacctcgtgtgtaaaaaggccgacaccgtgctgatcgtggacgtgaccgtcagatacgagatggacggcgagacactgaagagagccgccagcgagaaagtgaagcactatctgccagtgggccagcagatcaccgacaaagtcggcggacggtgcttcaaagtgatgggctttcctgtgggcgcaagaggcaaatggccagcctctaacaataccgtgctggccgaacttggagtgccagccggcagaatgaggacctttgctaggctggtgtcccggcggacactgctgtatagcctggacatcctgcgggacttcatgagagagcctgccggaagaggtacaagagtggcactgattccagctgccacaggcgctgctaacggatcctacccatacgatgttccagattacgcggccgctccaaaaaagaaaagaaaagttgaattcggcggcagctag

SEQ ID NO: 57 - DNA, 3624 bp, Artificial Sequence
  R2OI(ZF-Myb) with HA-tag and NLS- R2OI mutant, ZF1 and Myb N-terminal domains deleted
atgggcaccgacacagtgtacgtcggccaggattatcctagcggcctgagcaaaagagtgcccgctagactggttgctggccccatgctgagagagagatcttgtcacgcccacgtgttcagagccggacacatgtggaattggagaaccagcctgcctagcggcagatgggatcagcctgctctggaaaagtcccgggtgctgaccagatctgtggccaccgctacagaccccgagatcacatcttaccctggcaagagcgtgtccaccagcacacaggtgcaagaagaggactggtgtagcagagagagcggctggatttctcctggactggcccctgaggaacctagcgtggtgtctgagatcacagcctccatggtggccactatgagagtggctacagaggaagtggtgctggaacctcagcctgagcaggtcgtgacaattctgcccgagcacggcagaaatgtgccaccaggactggccgagcaggataccgcctctcctattgaagtgtccgtgctgctgcccgacctggccgaaaattgtcctctgtgtggtgttcccagcggcggactgagactgctgggaaagcactttgccgttagacatgccggcgtgcccgtgacctacgagtgtagaaagtgtgcctggcggagccccaatagccacagcatctcttgccacgtgccaaagtgcagaggcagagccagaatgccaagcggagatctgctgagaatcgataccgccagcaactctcccgacgacgccgaagtggaagaggaaagactggaatctctggccgtgcggtccagcagcagatctcctcctagtctggtggctaccagagtgcgggaagctgtggcaaggggagaatctgaaggcggcgaggaaatcagagccattgccgcactgatcagagatgtggatcagaacccctgcctgatcgagacaagcgccagcgacatcatcagcaagctgggcagaagagtggacggccctaaaagacccagacctgtcgtgcgggaacagacccaagaaaaaggctgggtccgacggctggccagacggaagagagagtatagagaggcccagtacctgtacagcagagatcaggcaagactggccgctcagattctggatggcgctgcctctcaagaatgcgccctgcctgtggatcaagtgtacggcgccttccgggaaaagtgggagacagtgggacagtttcacggcctgggcgagtttagaacaggcgctagagccgacaactgggagttctactctcccatcctggctgccgaagtcaaagaaaacctgatgcggatggccaacggcacagcccctggacctgatagaatcagcaagaaggccctgctggactgggaccctagaggcgaacagctggctagactgtacaccacatggctgatcggcggcgtgatccccagagtgttcaaagagtgtcggaccaagctgctgcctaagagcagcgatcctgtggaactgcaggatatcggaggatggcggcctgtgacaatcggcagcatggtcaccagactgttcagcagaatcctgaccatgcggctgacccgggcctgtcctatcaatcctagacagagaggcttcctggccagcagctctggatgtgccgagaacctgctgatcttcgacgagatcgtgcggcggtctagaagagatggtggaccactggccgtggtgttcgtggatttcgccagagccttcgacagcatcagccacgagcacatcctgtgtgttctggaagaaggcggcctggatagacacgtgatcggcctgattcggaacagctacgtggactgtgtgaccagagtgggctgcgtggaaggcatgacacctccaatccagatgaaggtcggagtgaagcagggcgaccctatgagccctctgctgttcaatctggctatggaccctctgattcacaagctggaaacagccggcacaggcctgaagtggggagatctgtctatcgccacactggccttcgccgatgatctggtgctggtgtcagacagcgaagaaggcatgggcagatccctgggcatcctggaaaaattctgccagctgaccggcctgagagtgcagcctagaaagtgccacggcttcttcatggacaagggcgtcgtgaatggctgcggcacatgggagatttgtggcagccctatccacatgatcccaccaggcgaatctgtgcgctatctgggcgttcaagttggccctggaagaggcgtgatggaacccgatctgatccctaccgtgcacacctggatcgagagaatctctgaggcccctctgaagcccagccagagaatgagagtgctgaatagcttcgccctgccacggatcatctatcaggctgacctgggcaaagtgaccgtgacaaagctggcccagatcgatggaattgtgcggaaagccgtgaagaagtggctgcatctgagccccagcacctgtaatggcctgctgtactccagaaacagagatggcggactggggctcctgaagctggaacgactgattcctagcgtgcggaccaagagaatctaccggatgagcagaagccccgacatctggaccagaagaatgaccagccactccgtgtccaagagcgactgggaaatgctgtgggtgcaagctggcggagaaagaggctctgctcctgttatgggagccgtggaagccgctcctaccgatgtggaaagatcccctgactaccccgattggcggagagaggaaaatcttgcttggagcgccctgagagttcaaggcgtgggagctgatcagttcagaggcgatagaacctccagcagctggatcgccgaacctgcctctgtgggatttgcccagagacattggctggctgctctggcacttagagccggcgtgtaccctaccagagagtttctggccaggggcaaagaaaagagcggagccgcctgtagaagatgccctgccagactggaaagctgcagccacatcctgggccagtgtcctttcgtgcaggccaacagaatcgcccggcacaacaaagtgtgcgtgctcctggcaaccgaggccgagagatttggctggaccgtgatccgggaattccggcttgaagatgctgctggcgggctgaagattcccgacctcgtgtgtaaaaaggccgacaccgtgctgatcgtggacgtgaccgtcagatacgagatggacggcgagacactgaagagagccgccagcgagaaagtgaagcactatctgccagtgggccagcagatcaccgacaaagtcggcggacggtgcttcaaagtgatgggctttcctgtgggcgcaagaggcaaatggccagcctctaacaataccgtgctggccgaacttggagtgccagccggcagaatgaggacctttgctaggctggtgtcccggcggacactgctgtatagcctggacatcctgcgggacttcatgagagagcctgccggaagaggtacaagagtggcactgattccagctgccacaggcgctgctaacggatcctacccatacgatgttccagattacgcggccgctccaaaaaagaaaagaaaagttgaattcggcggcagctag

SEQ ID NO: 58 - DNA, 3405 bp, Artificial Sequence
  R2(C114S, C117S, R151A, W152A) with HA-tag and NLS - R2 mutant, point mutations in catalytic residues of DNA-binding domain
atgatggccagcacagccctgtctctgatgggcagatgcaatcccgatggctgcacaagaggcaagcacgtgacagccgctcctatggatggacctagaggaccttctagcctggccggcacatttggatggggacttgctattcctgccggcgagccttgtggcagagtgtgttctcctgccaccgtgggattcttcccagtggccaagaagtccaacaaagagaacagacccgaggccagcggcctgcctctggaatctgaaagaaccggcgataatcctaccgtgcggggatctgctggtgccgatcctgttggacaagatgcccctggctggaccagccagttcagcgagagaaccttcagcaccaatagaggcctgggcgtgcacaaaagacgggctcaccctgtggaaacaaacaccgacgctgcccctatgatggtcaagagagccgcccacggcgaggaaatcgacctgctggccagaacagaagccagactgctggctgagaggggacagtgttctggcggagatctgtttggcgccctgcctggctttggaagaaccctggaagccatcaagggccagcgcagaagagagccttatagagccctggtgcaggcccacctggccagatttggatctcagcctggacctagctctggcggatgtagcgccgaacctgattttcggagagcctctggcgctgaagaggccggcgaagaaagatgtgctgaggatgccgccgcttacgatccttctgctgtgggccaaatgagccctgatgccgccagagtgctgtctgaacttcttgaaggcgctggcagacgcagagcctgtagagccatgaggcctaagaccgccggaagaagaaacgacctgcacgacgatagaaccgccagcgctcacaagaccagcagacagaagagaagggccgagtacgccagggtgcaagagctgtacaagaagtgcagatccagagccgccgctgaagtgattgatggtgcttgtggtggcgtgggccacagcctggaagagatggaaacctattggcggcccatcctggaaagagtgtctgacgctcctggaccaacacctgaagctctgcatgctctgggcagagctgagtggcatggcggcaatagagattacacccagctgtggaagcccatcagcgtggaagaaatcaaggccagcagattcgactggcggacaagccctggacctgatggcattagatctggacagtggcgggctgtgcctgtgcacctgaaggccgaaatgttcaacgcctggatggccagaggcgagatccctgagatcctgagacagtgcagaaccgtgttcgtgcccaaggtggaaagacctggcggaccaggcgagtacagacccatctctatcgccagcattcctctgcggcacttccactctatcctggctcggagacttctggcctgctgtcctcctgatgccagacagagaggctttatctgcgccgacggcaccctggaaaattctgcagtgctggatgccgtgctgggcgactctcggaagaaactgagagaatgtcacgtggccgtcctggacttcgccaaggcctttgatacagtgtctcacgaggccctggtggaactgctgagactgaggggaatgcctgagcagttctgtggctatatcgcccacctgtacgacaccgcctctaccacactggccgtgaacaatgagatgagcagccccgtgaaagttggcagaggcgttagacagggcgaccctctgagccccatcctgttcaatgtggtcatggatctgatcctggccagcctgcctgagagagtgggctatagactggaaatggaactggtgtctgccctggcctacgccgatgatctggttctgcttgccggcagcaaagtgggcatgcaagagtctatcagcgccgtggattgcgtgggcagacagatgggcctgcgcctgaattgcagaaaaagcgccgtgctgagcatgatccccgatggccacagaaagaagcaccactacctgaccgagcggaccttcaatatcggcggcaagcctctgagacaggtgtcctgtgttgagagatggcggtatctgggcgtcgactttgaggcctctggctgtgtgacactggaacactctatcagcagcgccctgaacaacatcagcagagcccctctgaagcctcagcagcggctggaaattctgagagcccatctgatccctcggttccagcacggatttgtgctgggcaacatctccgacgaccggctgagaatgctggacgtgcagatcagaaaagccgtcggccagtggctgagacttcctgcagatgtgcctaaggcctactatcacgctgctgtgcaagatggcggcctggctattccttctgtgcgcgccacaattcccgacctgatcgtgcgaagattcggcggacttgatagctctccttggagcgtggccagagctgccgccaagagcgataagatccggaaaaagctgcgctgggcctggaagcagctgcggagattttctagagtggacagcaccacacagcggcctagtgtgcggctgttttggagagaacatctgcacgcctccgtggacggcagagagctgagagaaagcaccagaacacccaccagcaccaagtggatcagagagagatgcgcccagatcacaggccgggatttcgtgcagttcgtgcacacccatatcaacgccctgccatccagaatcaggggcagcagaggtagaagaggcggaggcgaaagcagcctgacatgtagagccggctgtaaagtgcgcgagacaacagcccacatcctgcagcagtgtcatagaacacacggcggcagaatcctgcggcacaacaagattgtgtccttcgtggccaaggccatggaagagaacaagtggaccgtggaactggaacccagactgagaacaagcgtgggcctgagaaagcccgacatcattgcctctcgagatggcgtgggagtgatcgtggatgtgcaggttgtgtcaggccagagaagcctggacgagctgcacagagagaagcggaacaaatacggcaaccacggcgagctggttgaactggttgcaggcagactgggactgccaaaagccgagtgtgtgcgggccacctcttgtaccatttcttggagaggcgtgtggtccctgaccagctacaaagagctgcggtccatcatcggactgagagagcctacactgcagatcgtccccattctggccctgagaggcagccacatgaattggacccgcttcaaccagatgaccagcgtgatgggaggcggcgttggaggatcctacccatacgatgttccagattacgcggccgctccaaaaaagaaaagaaaagtttag

SEQ ID NO: 59 - DNA, 3138 bp, Artificial Sequence
  R2(ZF-Myb) with HA-tag and NLS - R2 mutant, ZF1 and Myb N-terminal domains deleted
atgatggccagcacagccctgtctctgatgggcagatgcaatcccgatggctgcacaagaggcaagcacgtgacagccgctcctatggatggacctagaggaccttctagcctggccggcacatttggatggggacttgctattcctgccggcgagccttgtggcagagtgtgttctcctgccaccgtgggattcttcccagtggccaagaagtccaacaaagagaacagacccgaggccagcggcctgcctctggaatctgaaagaaccggcgataatcctaccgtgcggggatctgctggtgccgatcctgttggacaagatgccagagagccttatagagccctggtgcaggcccacctggccagatttggatctcagcctggacctagctctggcggatgtagcgccgaacctgattttcggagagcctctggcgctgaagaggccggcgaagaaagatgtgctgaggatgccgccgcttacgatccttctgctgtgggccaaatgagccctgatgccgccagagtgctgtctgaacttcttgaaggcgctggcagacgcagagcctgtagagccatgaggcctaagaccgccggaagaagaaacgacctgcacgacgatagaaccgccagcgctcacaagaccagcagacagaagagaagggccgagtacgccagggtgcaagagctgtacaagaagtgcagatccagagccgccgctgaagtgattgatggtgcttgtggtggcgtgggccacagcctggaagagatggaaacctattggcggcccatcctggaaagagtgtctgacgctcctggaccaacacctgaagctctgcatgctctgggcagagctgagtggcatggcggcaatagagattacacccagctgtggaagcccatcagcgtggaagaaatcaaggccagcagattcgactggcggacaagccctggacctgatggcattagatctggacagtggcgggctgtgcctgtgcacctgaaggccgaaatgttcaacgcctggatggccagaggcgagatccctgagatcctgagacagtgcagaaccgtgttcgtgcccaaggtggaaagacctggcggaccaggcgagtacagacccatctctatcgccagcattcctctgcggcacttccactctatcctggctcggagacttctggcctgctgtcctcctgatgccagacagagaggctttatctgcgccgacggcaccctggaaaattctgcagtgctggatgccgtgctgggcgactctcggaagaaactgagagaatgtcacgtggccgtcctggacttcgccaaggcctttgatacagtgtctcacgaggccctggtggaactgctgagactgaggggaatgcctgagcagttctgtggctatatcgcccacctgtacgacaccgcctctaccacactggccgtgaacaatgagatgagcagccccgtgaaagttggcagaggcgttagacagggcgaccctctgagccccatcctgttcaatgtggtcatggatctgatcctggccagcctgcctgagagagtgggctatagactggaaatggaactggtgtctgccctggcctacgccgatgatctggttctgcttgccggcagcaaagtgggcatgcaagagtctatcagcgccgtggattgcgtgggcagacagatgggcctgcgcctgaattgcagaaaaagcgccgtgctgagcatgatccccgatggccacagaaagaagcaccactacctgaccgagcggaccttcaatatcggcggcaagcctctgagacaggtgtcctgtgttgagagatggcggtatctgggcgtcgactttgaggcctctggctgtgtgacactggaacactctatcagcagcgccctgaacaacatcagcagagcccctctgaagcctcagcagcggctggaaattctgagagcccatctgatccctcggttccagcacggatttgtgctgggcaacatctccgacgaccggctgagaatgctggacgtgcagatcagaaaagccgtcggccagtggctgagacttcctgcagatgtgcctaaggcctactatcacgctgctgtgcaagatggcggcctggctattccttctgtgcgcgccacaattcccgacctgatcgtgcgaagattcggcggacttgatagctctccttggagcgtggccagagctgccgccaagagcgataagatccggaaaaagctgcgctgggcctggaagcagctgcggagattttctagagtggacagcaccacacagcggcctagtgtgcggctgttttggagagaacatctgcacgcctccgtggacggcagagagctgagagaaagcaccagaacacccaccagcaccaagtggatcagagagagatgcgcccagatcacaggccgggatttcgtgcagttcgtgcacacccatatcaacgccctgccatccagaatcaggggcagcagaggtagaagaggcggaggcgaaagcagcctgacatgtagagccggctgtaaagtgcgcgagacaacagcccacatcctgcagcagtgtcatagaacacacggcggcagaatcctgcggcacaacaagattgtgtccttcgtggccaaggccatggaagagaacaagtggaccgtggaactggaacccagactgagaacaagcgtgggcctgagaaagcccgacatcattgcctctcgagatggcgtgggagtgatcgtggatgtgcaggttgtgtcaggccagagaagcctggacgagctgcacagagagaagcggaacaaatacggcaaccacggcgagctggttgaactggttgcaggcagactgggactgccaaaagccgagtgtgtgcgggccacctcttgtaccatttcttggagaggcgtgtggtccctgaccagctacaaagagctgcggtccatcatcggactgagagagcctacactgcagatcgtccccattctggccctgagaggcagccacatgaattggacccgcttcaaccagatgaccagcgtgatgggaggcggcgttggaggatcctacccatacgatgttccagattacgcggccgctccaaaaaagaaaagaaaagtttag

SEQ ID NO: 60 - DNA, 7857 bp, Artificial Sequence
  deadCas9-NLS-32aa-R2OI(ZF-Myb)-HA-NLS
atggacaagaagtatagcatcggcctggccatcggcacaaactccgtgggctgggccgtgatcaccgacgagtacaaggtgccaagcaagaagtttaaggtgctgggcaacaccgatagacactccatcaagaagaatctgatcggcgccctgctgttcgactctggcgagacagccgaggccacacggctgaagagaaccgcccggagaaggtatacacgccggaagaataggatctgctacctgcaggagatcttcagcaacgagatggccaaggtggacgattctttctttcaccgcctggaggagagcttcctggtggaggaggataagaagcacgagcggcaccctatctttggcaacatcgtggacgaggtggcctatcacgagaagtacccaacaatctatcacctgaggaagaagctggtggactccaccgataaggccgacctgcgcctgatctatctggccctggcccacatgatcaagttccggggccactttctgatcgagggcgatctgaacccagacaatagcgatgtggacaagctgttcatccagctggtgcagacctacaatcagctgtttgaggagaaccccatcaatgcctctggagtggacgcaaaggcaatcctgagcgccagactgtccaagtctagaaggctggagaacctgatcgcccagctgccaggcgagaagaagaacggcctgtttggcaatctgatcgccctgtccctgggcctgacacccaacttcaagtctaattttgatctggccgaggacgccaagctgcagctgtccaaggacacctatgacgatgacctggataacctgctggcccagatcggcgatcagtacgccgacctgttcctggccgccaagaatctgtctgacgccatcctgctgagcgatatcctgcgcgtgaacaccgagatcacaaaggcccccctgagcgcctccatgatcaagagatatgacgagcaccaccaggatctgaccctgctgaaggccctggtgaggcagcagctgcctgagaagtacaaggagatcttctttgatcagagcaagaatggatacgcaggatatatcgacggaggagcatcccaggaggagttctacaagtttatcaagcctatcctggagaagatggacggcacagaggagctgctggtgaagctgaatcgggaggacctgctgaggaagcagcgcacctttgataacggcagcatccctcaccagatccacctgggagagctgcacgcaatcctgcgccggcaggaggacttctacccatttctgaaggataaccgggagaagatcgagaagatcctgacattcagaatcccctactatgtgggacctctggcccggggcaatagcagatttgcctggatgacccgcaagtccgaggagacaatcacaccctggaacttcgaggaggtggtggataagggcgcctctgcccagagcttcatcgagcggatgaccaattttgacaagaacctgcctaatgagaaggtgctgccaaagcactctctgctgtacgagtatttcaccgtgtataacgagctgacaaaggtgaagtacgtgaccgagggcatgagaaagcctgccttcctgagcggcgagcagaagaaggccatcgtggacctgctgtttaagaccaataggaaggtgacagtgaagcagctgaaggaggactatttcaagaagatcgagtgttttgattctgtggagatcagcggcgtggaggacaggtttaacgcctccctgggcacctaccacgatctgctgaagatcatcaaggataaggacttcctggacaacgaggagaatgaggatatcctggaggacatcgtgctgaccctgacactgtttgaggatagggagatgatcgaggagcgcctgaagacatatgcccacctgttcgatgacaaagtgatgaagcagctgaagagaaggcgctacaccggatggggccggctgagcagaaagctgatcaatggcatccgcgacaagcagtctggcaagacaatcctggactttctgaagagcgatggcttcgccaaccggaacttcatgcagctgatccacgatgactccctgaccttcaaggaggatatccagaaggcacaggtgtctggacagggcgacagcctgcacgagcacatcgccaacctggccggctctcctgccatcaagaagggcatcctgcagaccgtgaaggtggtggacgagctggtgaaagtgatgggcaggcacaagccagagaacatcgtgatcgagatggcccgcgagaatcagaccacacagaagggccagaagaactcccgggagagaatgaagagaatcgaggagggcatcaaggagctgggctctcagatcctgaaggagcaccccgtggagaacacacagctgcagaatgagaagctgtatctgtactatctgcagaatggccgggatatgtacgtggaccaggagctggatatcaacagactgtctgattatgacgtggatcacatcgtgccacagtccttcctgaaggatgactctatcgacaataaggtgctgaccaggagcgacaaggcccgcggcaagtccgataatgtgccctctgaggaggtggtgaagaagatgaagaactactggaggcagctgctgaatgccaagctgatcacacagaggaagtttgataacctgaccaaggcagagaggggaggactgtccgagctggacaaggccggcttcatcaagcggcagctggtggagacaagacagatcacaaagcacgtggcccagatcctggattctagaatgaacacaaagtacgatgagaatgacaagctgatcagggaggtgaaagtgatcaccctgaagtccaagctggtgtctgactttaggaaggatttccagttttataaggtgcgcgagatcaacaattatcaccacgcccacgacgcctacctgaacgccgtggtgggcacagccctgatcaagaagtaccctaagctggagtccgagttcgtgtacggcgactataaggtgtacgatgtgcgcaagatgatcgccaagtctgagcaggagatcggcaaggccaccgccaagtatttcttttacagcaacatcatgaatttctttaagaccgagatcacactggccaatggcgagatcaggaagcgcccactgatcgagacaaacggcgagacaggcgagatcgtgtgggacaagggcagggattttgccaccgtgcgcaaggtgctgagcatgccccaagtgaatatcgtgaagaagaccgaggtgcagacaggcggcttctccaaggagtctatcctgcctaagcggaactccgataagctgatcgccagaaagaaggactgggaccccaagaagtatggcggcttcgacagccctacagtggcctactccgtgctggtggtggccaaggtggagaagggcaagagcaagaagctgaagtccgtgaaggagctgctgggcatcaccatcatggagcgcagctccttcgagaagaatcctatcgactttctggaggccaagggctataaggaggtgaagaaggacctgatcatcaagctgccaaagtactctctgtttgagctggagaacggaaggaagagaatgctggcaagcgccggagagctgcagaagggcaatgagctggccctgccctccaagtacgtgaacttcctgtatctggcctcccactacgagaagctgaagggctctcctgaggataacgagcagaagcagctgtttgtggagcagcacaagcactatctggacgagatcatcgagcagatcagcgagttctccaagagagtgatcctggccgacgccaatctggataaggtgctgtccgcctacaacaagcaccgggataagccaatcagagagcaggccgagaatatcatccacctgtttaccctgacaaacctgggagcaccagcagccttcaagtattttgacaccacaatcgacaggaagcggtacaccagcacaaaggaggtgctggacgccacactgatccaccagtccatcaccggcctgtacgagacacggatcgacctgtctcagctgggaggcgatggctccccaaaaaagaaaagaaaagttgctagctctggtggttcttctggtggttctagcggcagcgagactcccgggacctcagagtccgccacacccgaaagttctggtggttcttctggtggttctatgggcaccgacacagtgtacgtcggccaggattatcctagcggcctgagcaaaagagtgcccgctagactggttgctggccccatgctgagagagagatcttgtcacgcccacgtgttcagagccggacacatgtggaattggagaaccagcctgcctagcggcagatgggatcagcctgctctggaaaagtcccgggtgctgaccagatctgtggccaccgctacagaccccgagatcacatcttaccctggcaagagcgtgtccaccagcacacaggtgcaagaagaggactggtgtagcagagagagcggctggatttctcctggactggcccctgaggaacctagcgtggtgtctgagatcacagcctccatggtggccactatgagagtggctacagaggaagtggtgctggaacctcagcctgagcaggtcgtgacaattctgcccgagcacggcagaaatgtgccaccaggactggccgagcaggataccgcctctcctattgaagtgtccgtgctgctgcccgacctggccgaaaattgtcctctgtgtggtgttcccagcggcggactgagactgctgggaaagcactttgccgttagacatgccggcgtgcccgtgacctacgagtgtagaaagtgtgcctggcggagccccaatagccacagcatctcttgccacgtgccaaagtgcagaggcagagccagaatgccaagcggagatctgctgagaatcgataccgccagcaactctcccgacgacgccgaagtggaagaggaaagactggaatctctggccgtgcggtccagcagcagatctcctcctagtctggtggctaccagagtgcgggaagctgtggcaaggggagaatctgaaggcggcgaggaaatcagagccattgccgcactgatcagagatgtggatcagaacccctgcctgatcgagacaagcgccagcgacatcatcagcaagctgggcagaagagtggacggccctaaaagacccagacctgtcgtgcgggaacagacccaagaaaaaggctgggtccgacggctggccagacggaagagagagtatagagaggcccagtacctgtacagcagagatcaggcaagactggccgctcagattctggatggcgctgcctctcaagaatgcgccctgcctgtggatcaagtgtacggcgccttccgggaaaagtgggagacagtgggacagtttcacggcctgggcgagtttagaacaggcgctagagccgacaactgggagttctactctcccatcctggctgccgaagtcaaagaaaacctgatgcggatggccaacggcacagcccctggacctgatagaatcagcaagaaggccctgctggactgggaccctagaggcgaacagctggctagactgtacaccacatggctgatcggcggcgtgatccccagagtgttcaaagagtgtcggaccaagctgctgcctaagagcagcgatcctgtggaactgcaggatatcggaggatggcggcctgtgacaatcggcagcatggtcaccagactgttcagcagaatcctgaccatgcggctgacccgggcctgtcctatcaatcctagacagagaggcttcctggccagcagctctggatgtgccgagaacctgctgatcttcgacgagatcgtgcggcggtctagaagagatggtggaccactggccgtggtgttcgtggatttcgccagagccttcgacagcatcagccacgagcacatcctgtgtgttctggaagaaggcggcctggatagacacgtgatcggcctgattcggaacagctacgtggactgtgtgaccagagtgggctgcgtggaaggcatgacacctccaatccagatgaaggtcggagtgaagcagggcgaccctatgagccctctgctgttcaatctggctatggaccctctgattcacaagctggaaacagccggcacaggcctgaagtggggagatctgtctatcgccacactggccttcgccgatgatctggtgctggtgtcagacagcgaagaaggcatgggcagatccctgggcatcctggaaaaattctgccagctgaccggcctgagagtgcagcctagaaagtgccacggcttcttcatggacaagggcgtcgtgaatggctgcggcacatgggagatttgtggcagccctatccacatgatcccaccaggcgaatctgtgcgctatctgggcgttcaagttggccctggaagaggcgtgatggaacccgatctgatccctaccgtgcacacctggatcgagagaatctctgaggcccctctgaagcccagccagagaatgagagtgctgaatagcttcgccctgccacggatcatctatcaggctgacctgggcaaagtgaccgtgacaaagctggcccagatcgatggaattgtgcggaaagccgtgaagaagtggctgcatctgagccccagcacctgtaatggcctgctgtactccagaaacagagatggcggactggggctcctgaagctggaacgactgattcctagcgtgcggaccaagagaatctaccggatgagcagaagccccgacatctggaccagaagaatgaccagccactccgtgtccaagagcgactgggaaatgctgtgggtgcaagctggcggagaaagaggctctgctcctgttatgggagccgtggaagccgctcctaccgatgtggaaagatcccctgactaccccgattggcggagagaggaaaatcttgcttggagcgccctgagagttcaaggcgtgggagctgatcagttcagaggcgatagaacctccagcagctggatcgccgaacctgcctctgtgggatttgcccagagacattggctggctgctctggcacttagagccggcgtgtaccctaccagagagtttctggccaggggcaaagaaaagagcggagccgcctgtagaagatgccctgccagactggaaagctgcagccacatcctgggccagtgtcctttcgtgcaggccaacagaatcgcccggcacaacaaagtgtgcgtgctcctggcaaccgaggccgagagatttggctggaccgtgatccgggaattccggcttgaagatgctgctggcgggctgaagattcccgacctcgtgtgtaaaaaggccgacaccgtgctgatcgtggacgtgaccgtcagatacgagatggacggcgagacactgaagagagccgccagcgagaaagtgaagcactatctgccagtgggccagcagatcaccgacaaagtcggcggacggtgcttcaaagtgatgggctttcctgtgggcgcaagaggcaaatggccagcctctaacaataccgtgctggccgaacttggagtgccagccggcagaatgaggacctttgctaggctggtgtcccggcggacactgctgtatagcctggacatcctgcgggacttcatgagagagcctgccggaagaggtacaagagtggcactgattccagctgccacaggcgctgctaacggatcctacccatacgatgttccagattacgcggccgctccaaaaaagaaaagaaaagttgaattcggcggcagctag

SEQ ID NO: 61 - DNA, 8094 bp, Artificial Sequence
  NLS-R2OI (C248S, C251S, W294A)-HA-NLS-XTEN- deadCas9
gccaccatgcctaagaagaagagaaaggtgggtaccatgggcaccgacacagtgtacgtcggccaggattatcctagcggcctgagcaaaagagtgcccgctagactggttgctggccccatgctgagagagagatcttgtcacgcccacgtgttcagagccggacacatgtggaattggagaaccagcctgcctagcggcagatgggatcagcctgctctggaaaagtcccgggtgctgaccagatctgtggccaccgctacagaccccgagatcacatcttaccctggcaagagcgtgtccaccagcacacaggtgcaagaagaggactggtgtagcagagagagcggctggatttctcctggactggcccctgaggaacctagcgtggtgtctgagatcacagcctccatggtggccactatgagagtggctacagaggaagtggtgctggaacctcagcctgagcaggtcgtgacaattctgcccgagcacggcagaaatgtgccaccaggactggccgagcaggataccgcctctcctattgaagtgtccgtgctgctgcccgacctggccgaaaattgtcctctgtgtggtgttcccagcggcggactgagactgctgggaaagcactttgccgttagacatgccggcgtgcccgtgacctacgagtgtagaaagtgtgcctggcggagccccaatagccacagcatctcttgccacgtgccaaagtgcagaggcagagccagaatgccaagcggagatcctggaatcgccagcgatctgagcgaggccagatttgccacagaagtgggagtcgcccagcacaagagacacgtgcaccccgtggaatggaacaaagtgcggctggaaagaagaggcgccagaggcggaggaatcaaggccacaaaacttgccagcgtggccgaggtggaaaccctgatcagactgattagagagcacggcgatagcggcgccacataccagctgattgccgatgaactcggcagaggcaagacagccgagcaagtgcggagcaagaagcggctgctgagaatcgataccgccagcaactctcccgacgacgccgaagtggaagaggaaagactggaatctctggccgtgcggtccagcagcagatctcctcctagtctggtggctaccagagtgcgggaagctgtggcaaggggagaatctgaaggcggcgaggaaatcagagccattgccgcactgatcagagatgtggatcagaacccctgcctgatcgagacaagcgccagcgacatcatcagcaagctgggcagaagagtggacggccctaaaagacccagacctgtcgtgcgggaacagacccaagaaaaaggctgggtccgacggctggccagacggaagagagagtatagagaggcccagtacctgtacagcagagatcaggcaagactggccgctcagattctggatggcgctgcctctcaagaatgcgccctgcctgtggatcaagtgtacggcgccttccgggaaaagtgggagacagtgggacagtttcacggcctgggcgagtttagaacaggcgctagagccgacaactgggagttctactctcccatcctggctgccgaagtcaaagaaaacctgatgcggatggccaacggcacagcccctggacctgatagaatcagcaagaaggccctgctggactgggaccctagaggcgaacagctggctagactgtacaccacatggctgatcggcggcgtgatccccagagtgttcaaagagtgtcggaccaagctgctgcctaagagcagcgatcctgtggaactgcaggatatcggaggatggcggcctgtgacaatcggcagcatggtcaccagactgttcagcagaatcctgaccatgcggctgacccgggcctgtcctatcaatcctagacagagaggcttcctggccagcagctctggatgtgccgagaacctgctgatcttcgacgagatcgtgcggcggtctagaagagatggtggaccactggccgtggtgttcgtggatttcgccagagccttcgacagcatcagccacgagcacatcctgtgtgttctggaagaaggcggcctggatagacacgtgatcggcctgattcggaacagctacgtggactgtgtgaccagagtgggctgcgtggaaggcatgacacctccaatccagatgaaggtcggagtgaagcagggcgaccctatgagccctctgctgttcaatctggctatggaccctctgattcacaagctggaaacagccggcacaggcctgaagtggggagatctgtctatcgccacactggccttcgccgatgatctggtgctggtgtcagacagcgaagaaggcatgggcagatccctgggcatcctggaaaaattctgccagctgaccggcctgagagtgcagcctagaaagtgccacggcttcttcatggacaagggcgtcgtgaatggctgcggcacatgggagatttgtggcagccctatccacatgatcccaccaggcgaatctgtgcgctatctgggcgttcaagttggccctggaagaggcgtgatggaacccgatctgatccctaccgtgcacacctggatcgagagaatctctgaggcccctctgaagcccagccagagaatgagagtgctgaatagcttcgccctgccacggatcatctatcaggctgacctgggcaaagtgaccgtgacaaagctggcccagatcgatggaattgtgcggaaagccgtgaagaagtggctgcatctgagccccagcacctgtaatggcctgctgtactccagaaacagagatggcggactggggctcctgaagctggaacgactgattcctagcgtgcggaccaagagaatctaccggatgagcagaagccccgacatctggaccagaagaatgaccagccactccgtgtccaagagcgactgggaaatgctgtgggtgcaagctggcggagaaagaggctctgctcctgttatgggagccgtggaagccgctcctaccgatgtggaaagatcccctgactaccccgattggcggagagaggaaaatcttgcttggagcgccctgagagttcaaggcgtgggagctgatcagttcagaggcgatagaacctccagcagctggatcgccgaacctgcctctgtgggatttgcccagagacattggctggctgctctggcacttagagccggcgtgtaccctaccagagagtttctggccaggggcaaagaaaagagcggagccgcctgtagaagatgccctgccagactggaaagctgcagccacatcctgggccagtgtcctttcgtgcaggccaacagaatcgcccggcacaacaaagtgtgcgtgctcctggcaaccgaggccgagagatttggctggaccgtgatccgggaattccggcttgaagatgctgctggcgggctgaagattcccgacctcgtgtgtaaaaaggccgacaccgtgctgatcgtggacgtgaccgtcagatacgagatggacggcgagacactgaagagagccgccagcgagaaagtgaagcactatctgccagtgggccagcagatcaccgacaaagtcggcggacggtgcttcaaagtgatgggctttcctgtgggcgcaagaggcaaatggccagcctctaacaataccgtgctggccgaacttggagtgccagccggcagaatgaggacctttgctaggctggtgtcccggcggacactgctgtatagcctggacatcctgcgggacttcatgagagagcctgccggaagaggtacaagagtggcactgattccagctgccacaggcgctgctaacggatcctacccatacgatgttccagattacgcggccgctccaaaaaagaaaagaaaagttgaattcggcggcagcagcggcagcgagactcccgggacctcagagtccgccacacccgaaagtatggacaagaagtatagcatcggcctggccatcggcacaaactccgtgggctgggccgtgatcaccgacgagtacaaggtgccaagcaagaagtttaaggtgctgggcaacaccgatagacactccatcaagaagaatctgatcggcgccctgctgttcgactctggcgagacagccgaggccacacggctgaagagaaccgcccggagaaggtatacacgccggaagaataggatctgctacctgcaggagatcttcagcaacgagatggccaaggtggacgattctttctttcaccgcctggaggagagcttcctggtggaggaggataagaagcacgagcggcaccctatctttggcaacatcgtggacgaggtggcctatcacgagaagtacccaacaatctatcacctgaggaagaagctggtggactccaccgataaggccgacctgcgcctgatctatctggccctggcccacatgatcaagttccggggccactttctgatcgagggcgatctgaacccagacaatagcgatgtggacaagctgttcatccagctggtgcagacctacaatcagctgtttgaggagaaccccatcaatgcctctggagtggacgcaaaggcaatcctgagcgccagactgtccaagtctagaaggctggagaacctgatcgcccagctgccaggcgagaagaagaacggcctgtttggcaatctgatcgccctgtccctgggcctgacacccaacttcaagtctaattttgatctggccgaggacgccaagctgcagctgtccaaggacacctatgacgatgacctggataacctgctggcccagatcggcgatcagtacgccgacctgttcctggccgccaagaatctgtctgacgccatcctgctgagcgatatcctgcgcgtgaacaccgagatcacaaaggcccccctgagcgcctccatgatcaagagatatgacgagcaccaccaggatctgaccctgctgaaggccctggtgaggcagcagctgcctgagaagtacaaggagatcttctttgatcagagcaagaatggatacgcaggatatatcgacggaggagcatcccaggaggagttctacaagtttatcaagcctatcctggagaagatggacggcacagaggagctgctggtgaagctgaatcgggaggacctgctgaggaagcagcgcacctttgataacggcagcatccctcaccagatccacctgggagagctgcacgcaatcctgcgccggcaggaggacttctacccatttctgaaggataaccgggagaagatcgagaagatcctgacattcagaatcccctactatgtgggacctctggcccggggcaatagcagatttgcctggatgacccgcaagtccgaggagacaatcacaccctggaacttcgaggaggtggtggataagggcgcctctgcccagagcttcatcgagcggatgaccaattttgacaagaacctgcctaatgagaaggtgctgccaaagcactctctgctgtacgagtatttcaccgtgtataacgagctgacaaaggtgaagtacgtgaccgagggcatgagaaagcctgccttcctgagcggcgagcagaagaaggccatcgtggacctgctgtttaagaccaataggaaggtgacagtgaagcagctgaaggaggactatttcaagaagatcgagtgttttgattctgtggagatcagcggcgtggaggacaggtttaacgcctccctgggcacctaccacgatctgctgaagatcatcaaggataaggacttcctggacaacgaggagaatgaggatatcctggaggacatcgtgctgaccctgacactgtttgaggatagggagatgatcgaggagcgcctgaagacatatgcccacctgttcgatgacaaagtgatgaagcagctgaagagaaggcgctacaccggatggggccggctgagcagaaagctgatcaatggcatccgcgacaagcagtctggcaagacaatcctggactttctgaagagcgatggcttcgccaaccggaacttcatgcagctgatccacgatgactccctgaccttcaaggaggatatccagaaggcacaggtgtctggacagggcgacagcctgcacgagcacatcgccaacctggccggctctcctgccatcaagaagggcatcctgcagaccgtgaaggtggtggacgagctggtgaaagtgatgggcaggcacaagccagagaacatcgtgatcgagatggcccgcgagaatcagaccacacagaagggccagaagaactcccgggagagaatgaagagaatcgaggagggcatcaaggagctgggctctcagatcctgaaggagcaccccgtggagaacacacagctgcagaatgagaagctgtatctgtactatctgcagaatggccgggatatgtacgtggaccaggagctggatatcaacagactgtctgattatgacgtggatcacatcgtgccacagtccttcctgaaggatgactctatcgacaataaggtgctgaccaggagcgacaaggcccgcggcaagtccgataatgtgccctctgaggaggtggtgaagaagatgaagaactactggaggcagctgctgaatgccaagctgatcacacagaggaagtttgataacctgaccaaggcagagaggggaggactgtccgagctggacaaggccggcttcatcaagcggcagctggtggagacaagacagatcacaaagcacgtggcccagatcctggattctagaatgaacacaaagtacgatgagaatgacaagctgatcagggaggtgaaagtgatcaccctgaagtccaagctggtgtctgactttaggaaggatttccagttttataaggtgcgcgagatcaacaattatcaccacgcccacgacgcctacctgaacgccgtggtgggcacagccctgatcaagaagtaccctaagctggagtccgagttcgtgtacggcgactataaggtgtacgatgtgcgcaagatgatcgccaagtctgagcaggagatcggcaaggccaccgccaagtatttcttttacagcaacatcatgaatttctttaagaccgagatcacactggccaatggcgagatcaggaagcgcccactgatcgagacaaacggcgagacaggcgagatcgtgtgggacaagggcagggattttgccaccgtgcgcaaggtgctgagcatgccccaagtgaatatcgtgaagaagaccgaggtgcagacaggcggcttctccaaggagtctatcctgcctaagcggaactccgataagctgatcgccagaaagaaggactgggaccccaagaagtatggcggcttcgacagccctacagtggcctactccgtgctggtggtggccaaggtggagaagggcaagagcaagaagctgaagtccgtgaaggagctgctgggcatcaccatcatggagcgcagctccttcgagaagaatcctatcgactttctggaggccaagggctataaggaggtgaagaaggacctgatcatcaagctgccaaagtactctctgtttgagctggagaacggaaggaagagaatgctggcaagcgccggagagctgcagaagggcaatgagctggccctgccctccaagtacgtgaacttcctgtatctggcctcccactacgagaagctgaagggctctcctgaggataacgagcagaagcagctgtttgtggagcagcacaagcactatctggacgagatcatcgagcagatcagcgagttctccaagagagtgatcctggccgacgccaatctggataaggtgctgtccgcctacaacaagcaccgggataagccaatcagagagcaggccgagaatatcatccacctgtttaccctgacaaacctgggagcaccagcagccttcaagtattttgacaccacaatcgacaggaagcggtacaccagcacaaaggaggtgctggacgccacactgatccaccagtccatcaccggcctgtacgagacacggatcgacctgtctcagctgggaggcgattga

SEQ ID NO: 62 - DNA, 21 bp, Artificial Sequence
  Primer A
ctcaggtagtggttgtcgggc

SEQ ID NO: 63 - DNA, 20 bp, Artificial Sequence
  Primer B
ggacagtgggaatctcgttc

SEQ ID NO: 64 - DNA, 18 bp, Artificial Sequence
  Primer C
tgggagtctcggcatgat

SEQ ID NO: 65 - DNA, 67 bp, Artificial Sequence
  Sequence of Fig. 2 - Band 1 - Non-spliced - using Primer A
ggggtgttctgctggtagtggtcggcgaggtgagtccaggagatgtttcagccatgttgtctttatt

SEQ ID NO: 66 - DNA, 932 bp, Artificial Sequence
  Sequence of Fig. 2 - Band 1 - Non-spliced - using Primer B
agatgacgaggcatttggctacttgaggcgagtcaccactcgctttccggattaatgtgtccgtcacggggacgacatccgagtgcagcgcaagatttgtaatcatgccgagactcccagctgtcccccgggttgcgccttttccaaggcagccctgggtttgcgcagggacgcggctgctctgggcgtggttccgggaaacgcagcggcgccgaccctgggtctcgcacattcttcacgtccgttcgcagcgtcacccggatcttcgccgctacccttgtgggccccccggcgacgcttcctgctccgcccctaagtcgggaaggttccttgcggttcgcggcgtgccggacgtgacaaacggaagccgcacgtctcactagtaccctcgcagacggacagcgccagggagcaatggcagcgcgccgaccgcgatgggctgtggccaatagcggctgctcagcagggcgcgccgagagcagcggccgggaaggggcggtgcgggaggcggggtgtggggctgtagtgtgggccctgttcctgcccgcgcggtgttccgcattctgcaagcctccggagcgcacgtcggcagtcggctccctcgttgaccgaatcaccgacctctctccccaggcaagtttgtacaaaaaagcaggctgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgc

SEQ ID NO: 67 - DNA, 898 bp, Artificial Sequence
  Sequence of Fig. 2 - Band 2 - Spliced - using Primer A
tgggggtgttctgctggtagtggtcggcgagctgcacgctgccgtcctcgatgttgtggcggatcttgaagttcaccttgatgccgttcttctgcttgtcggccatgatatagacgttgtggctgttgtagttgtactccagcttgtgccccaggatgttgccgtcctccttgaagtcgatgcccttcagctcgatgcggttaaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcggctgaagcactgcacgccgtaggtcagggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccatggtggcagcctgcttttttgtacaaacttgcctggggagagaggtcggtgattcggtcaacgagggagccgactgccgacgtgcgctccggaggcttgcagaatgcggaacaccgcgcgggcaggaacagggcccacactacagccccacaccccgcctcccgcaccgccccttcccggccgctgctctcggcgcgccctgctgagcagccgctattggccacagcccatcgcggtcggcgcgctgccattgctccctggcgctgtccgtctgcgagggtactagtgagacgtgcggcttccgtttgtcacgtccggcacgccgcgaaccgca

SEQ ID NO: 68 - DNA, 971 bp, Artificial Sequence
  Sequence of Fig. 2 - Band 2 - Spliced - using Primer B
  misc_feature (737)..(737): n is a, c, g, or t
  misc_feature (788)..(788): n is a, c, g, or t
  misc_feature (868)..(868): n is a, c, g, or t
  misc_feature (941)..(941): n is a, c, g, or t
ttagatgacgaggcatttggctacttgaggcgagtcaccactcgctttccggattaatgtgtccgtcacggggacgacatccgagtgcagcgcaagatttgtaatcatgccgagactcccagctgtcccccgggttgcgccttttccaaggcagccctgggtttgcgcagggacgcggctgctctgggcgtggttccgggaaacgcagcggcgccgaccctgggtctcgcacattcttcacgtccgttcgcagcgtcacccggatcttcgccgctacccttgtgggccccccggcgacgcttcctgctccgcccctaagtcgggaaggttccttgcggttcgcggcgtgccggacgtgacaaacggaagccgcacgtctcactagtaccctcgcagacggacagcgccagggagcaatggcagcgcgccgaccgcgatgggctgtggccaatagcggctgctcagcagggcgcgccgagagcagcggccgggaaggggcggtgcgggaggcggggtgtggggctggtagtgtgggccctgttcctgcccgcgcggtgttccgcattctgcaagcctccggagcgcacgtcggcagtcggctccctcgttgaccgaatcaccgacctctctccccaggcaagtttgtacaaaaaagcaggctgccaccatggtgagcaagggcgaggagctgttcaccggggtggtgcccatcctggtcgagctggacggcgacgtanacggccacaagttcagcgtgtccggcgagggcgagggcgatgccacctacngcaagctgaccctgaagttcatctgcaccaccggcaagctgcccgtgccctggcccaccctcgtgaccaccctgacctanggcgtgcagtgcttcagccgctaccccgaccacatgaagcagcacgacttcttcaagtccgccatgcccgaanctacgtccaggagcgcaccatcttcttcaa

SEQ ID NO: 69 - DNA, 118 bp, Artificial Sequence
  Sequence of Fig. 2 - Band 2 - Spliced - using Primer C
gtcgtcccgtgacggacacattaatccggaaagcgagtggtgactcgcctcaagtagccaaatgcctcgtcatctaattagtgacgcgcatgaatggatgaacgagattcccactgtc

SEQ ID NO: 70 - DNA, 20 bp, Artificial Sequence
  Example 4 - Forward Primer
tgctcaggtagtggttgtcg

SEQ ID NO: 71 - DNA, 3188 bp, Artificial Sequence
  R2OI_EGFP_reporter RNA sequence
gcgggtgttgacgcgatgtgatttctgcccagtgctctgaatgtcaaagtgaagaaattcaatgaagcgcgggtaaacggcgggagtaactatgactctcttaaggcgcacaggggacacagagcctgcccaagtaccgctcccgagggagcgggaaacgggggggtgactatcccctggggtccggcgagagcgctggtctacggaccaggggtggctgtgggcaggctgctcctcaggccagttgattagttacgcatgggctgtacctccacgtggtcccgctggtaacgacttgtcggctaaatcagcccgcccaccatctgggatatggttgaccgtctaaccccagtactcaggtcacaaacaaaatgggaacagatacagtgtatgtcggccaggactacccttctggcttatcaaaacgggtaccagcacggttagtggcgggaccgatgctgcgagagcgaagctgtcacgcccatgtgtttagggctggacacatgtggaactggcgaaccagccttccgagcgggcgctgggaccagcccgctttggagaagtctcgggtcctaacccggtcggtggcgacggccaccgaccccgaaattacctcttacccaggaaagtccgtatcgacaagtacgcaggttcaggaggaggactggtgtagccgggagagcgggtggatctcgccaggacttgctcctgaagaaccctcggtggtgtccgaaattacagcctccatggtagcgacaatgagggtagcaaccgaggaggtcgtgtaagatacattgatgagtttggacaaaccacaactagaatgcagtgaaaaaaatgctttatttgtgaaatttgtgatgctattgctttatttgtaaccattataagctgcaataaacaagttgtttttacttgtacagctcgtccatgccgagagtgatcccggcggcggtcacgaactccagcaggaccatgtgatcgcgcttctcgttggggtctttgctcagggcggactgggtgctcaggtagtggttgtcgggcagcagcacggggccgtcgccgatgggggtgttctgctggtagtggtcggcgaggtgagtccaggagatgtttcagcactgttgcctttagtctcgaggcaacttagacaactgagtattgatctgagcacagcagggtgtgagctgtttgaagatactggggttgggagtgaagaaactgcagaggactaactgggctgagacccagtggcaatgttttagggcctaaggagtgcctctgaaaatctagatggacaactttgactttgagaaaagagaggtggaaatgaggaaaatgacttttctttattagatttcggtagaaagaactttcacctttcccctatttttgttattcgttttaaaacatctatctggaggcaggacaagtatggtcgttaaaaagatgcaggcagaaggcatatattggctcagtcaaagtggggaactttggtggccaaacatacattgctaaggctattcctatatcagctggacacatataaaatgctgctaatgcttcattacaaacttatatcctttaattccagatgggggcaaagtatgtccaggggtgaggaacaattgaaacatttgggctggagtagattttgaaagtcagctctgtgtgtgtgtgtgtgtgtgcgcgcgcgtgtgtgtgtgtgtgtgtcagcgtgtgtttcttttaacgtcttcagcctacaacatacagggttcatggtgggaagaagatagcaagatttaaattatggccagtgactagtgctgcaagaagaacaactacctgcatttaatgggaaagcaaaatctcaggctttgagggaagttaacataggcttgattctgggttgaagctgggtgtgtagttatctggaggccaggctggagctctcagctcactatgggttcatctttattgtctcctttcatctcaacagctgcacgctgccgtcctcgatgttgtggcggatcttgaagttcaccttgatgccgttcttctgcttgtcggccatgatatagacgttgtggctgttgtagttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcggctgaagcactgcacgccgtaggtcagggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccatggtggcagcctgcttttttgtacaaacttgcctggggagagaggtcggtgattcggtcaacgagggagccgactgccgacgtgcgctccggaggcttgcagaatgcggaacaccgcgcgggcaggaacagggcccacactaccgccccacaccccgcctcccgcaccgccccttcccggccgctgctctcggcgcgccctgctgagcagccgctattggccacagcccatcgcggtcggcgcgctgccattgctccctggcgctgtccgtctgcgagggtactagtgagacgtgcggcttccgtttgtcacgtccggcacgccgcgaaccgcaaggaaccttcccgacttaggggcggagcaggaagcgtcgccggggggcccacaagggtagcggcgaagatccgggtgacgctgcgaacggacgtgaagaatgtgcgagacccagggtcggcgccgctgcgtttcccggaaccacgcccagagcagccgcgtccctgcgcaaacccagggctgccttggaaaaggcgcaacccgggggacagctgggagtctcggcatgattacaaatcttgcgctgcactcggatgtcgtccccgtgacggacacattaatccggaaagcgagtggtgactcgcctcaagtagccaaatgcctcgtcatctaattagtga

SEQ ID NO: 72 - DNA, 2265 bp, Artificial Sequence
  EGFP cassette sequence
taagatacattgatgagtttggacaaaccacaactagaatgcagtgaaaaaaatgctttatttgtgaaatttgtgatgctattgctttatttgtaaccattataagctgcaataaacaagttgtttttacttgtacagctcgtccatgccgagagtgatcccggcggcggtcacgaactccagcaggaccatgtgatcgcgcttctcgttggggtctttgctcagggcggactgggtgctcaggtagtggttgtcgggcagcagcacggggccgtcgccgatgggggtgttctgctggtagtggtcggcgaggtgagtccaggagatgtttcagcactgttgcctttagtctcgaggcaacttagacaactgagtattgatctgagcacagcagggtgtgagctgtttgaagatactggggttgggagtgaagaaactgcagaggactaactgggctgagacccagtggcaatgttttagggcctaaggagtgcctctgaaaatctagatggacaactttgactttgagaaaagagaggtggaaatgaggaaaatgacttttctttattagatttcggtagaaagaactttcacctttcccctatttttgttattcgttttaaaacatctatctggaggcaggacaagtatggtcgttaaaaagatgcaggcagaaggcatatattggctcagtcaaagtggggaactttggtggccaaacatacattgctaaggctattcctatatcagctggacacatataaaatgctgctaatgcttcattacaaacttatatcctttaattccagatgggggcaaagtatgtccaggggtgaggaacaattgaaacatttgggctggagtagattttgaaagtcagctctgtgtgtgtgtgtgtgtgtgcgcgcgcgtgtgtgtgtgtgtgtgtcagcgtgtgtttcttttaacgtcttcagcctacaacatacagggttcatggtgggaagaagatagcaagatttaaattatggccagtgactagtgctgcaagaagaacaactacctgcatttaatgggaaagcaaaatctcaggctttgagggaagttaacataggcttgattctgggttgaagctgggtgtgtagttatctggaggccaggctggagctctcagctcactatgggttcatctttattgtctcctttcatctcaacagctgcacgctgccgtcctcgatgttgtggcggatcttgaagttcaccttgatgccgttcttctgcttgtcggccatgatatagacgttgtggctgttgtagttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcggctgaagcactgcacgccgtaggtcagggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccatggtggcagcctgcttttttgtacaaacttgcctggggagagaggtcggtgattcggtcaacgagggagccgactgccgacgtgcgctccggaggcttgcagaatgcggaacaccgcgcgggcaggaacagggcccacactaccgccccacaccccgcctcccgcaccgccccttcccggccgctgctctcggcgcgccctgctgagcagccgctattggccacagcccatcgcggtcggcgcgctgccattgctccctggcgctgtccgtctgcgagggtactagtgagacgtgcggcttccgtttgtcacgtccggcacgccgcgaaccgcaaggaaccttcccgacttaggggcggagcaggaagcgtcgccggggggcccacaagggtagcggcgaagatccgggtgacgctgcgaacggacgtgaagaatgtgcgagacccagggtcggcgccgctgcgtttcccggaaccacgcccagagcagccgcgtccctgcgcaaacccagggctgccttggaaaaggcgcaaccc